SEQUENCE LISTING (62 sequences)

SEQ ID NO: 1 - protein, 121 aa, Synthetic construct
QVQLVQSGTEVRKPGSSVKVSCKTSGVTFNNYAITWVRQAPGQGLEWMGGIIPVFSPPNYAQKFQGRVTITADESTNTAYMELNSLRSDDTAIYFCAREGERGYTYGYDYWGQGTLVTVSS

SEQ ID NO: 2 - protein, 8 aa, Synthetic construct
GVTFNNYA

SEQ ID NO: 3 - protein, 8 aa, Synthetic construct
IIPVFSPP

SEQ ID NO: 4 - protein, 14 aa, Synthetic construct
AREGERGYTYGYDY

SEQ ID NO: 5 - protein, 108 aa, Synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 6 - protein, 6 aa, Synthetic construct
QSISSY

SEQ ID NO: 7 - skipped

SEQ ID NO: 8 - protein, 10 aa, Synthetic construct
QQSYSTPPIT

SEQ ID NO: 9 - protein, 447 aa, Synthetic construct
QVQLVQSGTEVRKPGSSVKVSCKTSGVTFNNYAITWVRQAPGQGLEWMGGIIPVFSPPNYAQKFQGRVTITADESTNTAYMELNSLRSDDTAIYFCAREGERGYTYGYDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 215 aa, Synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 117 aa, Synthetic construct [sequence omitted]

SEQ ID NO: 12 - protein, 8 aa, Synthetic construct
GFTFSNFG

SEQ ID NO: 13 - protein, 8 aa, Synthetic construct
ISGGGRDT

SEQ ID NO: 14 - protein, 10 aa, Synthetic construct
VKWGNIYFDY

SEQ ID NO: 15 - protein, 107 aa, Synthetic construct [sequence omitted]

SEQ ID NO: 16 - protein, 6 aa, Synthetic construct
LSINTF

SEQ ID NO: 17 - protein, 9 aa, Synthetic construct
QQSSNTPFT

SEQ ID NO: 18 - protein, 443 aa, Synthetic construct
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - protein, 214 aa, Synthetic construct
DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 121 aa, Synthetic construct
QVQLVQSGAEVKRPGSSVKVSCKVSGVTFRNFAIIWVRQAPGQGLEWMGGIIPFFSAANYAQSFQGRVTITPDESTSTAFMELASLRSEDTAVYYCAREGERGHTYGFDYWGQGTLVTVSS

SEQ ID NO: 21 - protein, 8 aa, Synthetic construct
GVTFRNFA

SEQ ID NO: 22 - protein, 8 aa, Synthetic construct
IIPFFSAA

SEQ ID NO: 23 - protein, 14 aa, Synthetic construct
AREGERGHTYGFDY

SEQ ID NO: 24 - protein, 447 aa, Synthetic construct
QVQLVQSGAEVKRPGSSVKVSCKVSGVTFRNFAIIWVRQAPGQGLEWMGGIIPFFSAANYAQSFQGRVTITPDESTSTAFMELASLRSEDTAVYYCAREGERGHTYGFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 25 - protein, 215 aa, Synthetic construct; identical to SEQ ID NO: 10

SEQ ID NO: 26 - protein, 326 aa, Synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - protein, 323 aa, Synthetic construct
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGGGGGSGGGGSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 28 - protein, 165 aa, Homo sapiens
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTG

SEQ ID NO: 29 - protein, 133 aa, Homo sapiens [sequence omitted]

SEQ ID NO: 30 - protein, 15 aa, Synthetic construct
GGGGSGGGGSGGGGS

SEQ ID NO: 31 - protein, 25 aa, Synthetic construct
GGGGSGGGGSGGGGSGGGGSGGGGS

SEQ ID NO: 32 - protein, 5 aa, Synthetic construct
GGGGS

SEQ ID NO: 33 - DNA, 399 bp, Synthetic construct
  source 1..399: mol_type = other DNA
atgggaattcgcttgctctgtcgcgtcgctttctgttttctcgccgtcggacttgtggatgtcaaggtcacccagtcctcccgctacctggtcaagaggactggagagaaagtgttcctggaatgcgtgcaggacatggaccatgaaaacatgttctggtatagacaggaccccgggctgggactgcggctgatctacttctcctacgacgtgaagatgaaggaaaagggcgacatccctgagggatactcagtgtcaagagagaagaaggagcggttctcccttatcctggaatccgcctcgactaatcagacctcgatgtacctgtgcgcgtcctcctttaccggtccttacaactcccccctgcacttcgggaatggcacccggctgactgtgacc

SEQ ID NO: 34 - DNA, 531 bp, Synthetic construct
  source 1..531: mol_type = other DNA
gaagatctcaacaaagtgtttcctccggaagtggcagtcttcgagccatccgaagccgagatcagccacactcagaaggccaccctggtctgcttggctaccggattcttccctgaccacgtggaactttcttggtgggtgaacggaaaagaagtccactccggagtctccactgaccctcagccgctgaaggaacagccggccttgaacgactcgcgctactgcctgtcctcccggctgagagtgtccgccacgttctggcaaaacccgaggaaccatttccggtgccaagtgcagttctacggactcagcgagaacgacgagtggacccaggacagggcaaagcccgtgactcaaatcgtgtccgccgaagcctggggacgggctgattgcggcttcaccagcgtgtcatatcagcaaggagtgctgtcggccactatcctctacgagattctcttgggcaaagcaacactgtacgcggtgctcgtcagcgccctggtgctgatggccatggtcaagcgcaaggacttt

SEQ ID NO: 35 - skipped

SEQ ID NO: 36 - DNA, 54 bp, Synthetic construct
  source 1..54: mol_type = other DNA
gagggcagaggaagtcttctaacatgcggtgacgtggaggagaatcccggccct

SEQ ID NO: 37 - DNA, 717 bp, Synthetic construct
  source 1..717: mol_type = other DNA
atggtgagcaagggagaggagctgttcaccggagtggtgccaatcctggtggagctggacggcgatgtgaatggccacaagtttagcgtgtccggagagggagagggcgacgcaacatacggcaagctgaccctgaagttcatctgcacaaccggcaagctgcctgtgccatggcccacactggtgacaaccctgacctacggcgtgcagtgtttctctagatatccagatcacatgaagcagcacgacttctttaagagcgccatgccagagggatacgtgcaggagcgcaccatcttctttaaggacgatggcaactataagacacgggccgaggtgaagttcgagggcgataccctggtgaacagaatcgagctgaagggcatcgacttcaaggaggacggcaatatcctgggccacaagctggagtacaactataatagccacaacgtgtacatcatggccgacaagcagaagaacggcatcaaggtgaacttcaagatccggcacaatatcgaggatggctccgtgcagctggccgaccactaccagcagaacacaccaatcggcgatggcccagtgctgctgcccgacaatcactatctgtctacccagagcgccctgtccaaggatcccaacgagaagagagaccacatggtgctgctggagttcgtgacagcagcaggaatcaccctgggaatggacgagctgtataag

SEQ ID NO: 38 - DNA, 12 bp, Synthetic construct
  source 1..12: mol_type = other DNA
cgggccaagcgc

SEQ ID NO: 39 - DNA, 57 bp, Synthetic construct
  source 1..57: mol_type = other DNA
gcgactaacttttccctgctgaagcaggctggcgatgtggaagagaaccctgggcca

SEQ ID NO: 40 - DNA, 411 bp, Synthetic construct
  source 1..411: mol_type = other DNA
atgtccctgagcagcctgctgaaggtcgtgaccgcgtcattgtggctgggaccgggcattgcccagaagatcacccagacccagccggggatgtttgtgcaagaaaaggaagccgttaccctcgactgcacttacgacacctccgacccgtcatacggactgttctggtacaagcaacccagcagcggagaaatgatcttcctgatctaccaagggtcctacgaccagcagaatgctaccgaaggtcgctacagcctgaatttccagaaggcccgcaagagcgccaacctcgtgatttctgcctcccaactcggcgattccgcaatgtacttctgtgcgatgcggggtggcggctccggcggcagctacatccccaccttcggtcggggcacctcactgattgtgcaccca

SEQ ID NO: 41 - DNA, 423 bp, Synthetic construct
  source 1..423: mol_type = other DNA
tacatccagaatccggatcctgcggtctatcaattaagggactccaagtcttccgataaatccgtgtgtctctttacagacttcgactcgcaaaccaacgtgtcccagtcaaaggactcggatgtgtacatcaccgacaagactgtgctggacatgcggtcgatggacttcaagtccaacagcgcggtggcctggtccaacaagagcgacttcgcctgtgcgaacgccttcaacaactccatcattcccgaggacaccttcttcccatcccctgagtcctcctgcgacgtgaagctcgtggagaagtcgttcgagactgataccaacctgaactttcaaaacctgagcgtgatagggttcaggatcctgttactcaaagtcgccggtttcaacctcctgatgaccctgagactttggtcaagt

SEQ ID NO: 42 - DNA, 2622 bp, Synthetic construct
  source 1..2622: mol_type = other DNA
atgggaattcgcttgctctgtcgcgtcgctttctgttttctcgccgtcggacttgtggatgtcaaggtcacccagtcctcccgctacctggtcaagaggactggagagaaagtgttcctggaatgcgtgcaggacatggaccatgaaaacatgttctggtatagacaggaccccgggctgggactgcggctgatctacttctcctacgacgtgaagatgaaggaaaagggcgacatccctgagggatactcagtgtcaagagagaagaaggagcggttctcccttatcctggaatccgcctcgactaatcagacctcgatgtacctgtgcgcgtcctcctttaccggtccttacaactcccccctgcacttcgggaatggcacccggctgactgtgaccgaagatctcaacaaagtgtttcctccggaagtggcagtcttcgagccatccgaagccgagatcagccacactcagaaggccaccctggtctgcttggctaccggattcttccctgaccacgtggaactttcttggtgggtgaacggaaaagaagtccactccggagtctccactgaccctcagccgctgaaggaacagccggccttgaacgactcgcgctactgcctgtcctcccggctgagagtgtccgccacgttctggcaaaacccgaggaaccatttccggtgccaagtgcagttctacggactcagcgagaacgacgagtggacccaggacagggcaaagcccgtgactcaaatcgtgtccgccgaagcctggggacgggctgattgcggcttcaccagcgtgtcatatcagcaaggagtgctgtcggccactatcctctacgagattctcttgggcaaagcaacactgtacgcggtgctcgtcagcgccctggtgctgatggccatggtcaagcgcaaggactttggatccggagagggcagaggaagtcttctaacatgcggtgacgtggaggagaatcccggccctatggtgagcaagggagaggagctgttcaccggagtggtgccaatcctggtggagctggacggcgatgtgaatggccacaagtttagcgtgtccggagagggagagggcgacgcaacatacggcaagctgaccctgaagttcatctgcacaaccggcaagctgcctgtgccatggcccacactggtgacaaccctgacctacggcgtgcagtgtttctctagatatccagatcacatgaagcagcacgacttctttaagagcgccatgccagagggatacgtgcaggagcgcaccatcttctttaaggacgatggcaactataagacacgggccgaggtgaagttcgagggcgataccctggtgaacagaatcgagctgaagggcatcgacttcaaggaggacggcaatatcctgggccacaagctggagtacaactataatagccacaacgtgtacatcatggccgacaagcagaagaacggcatcaaggtgaacttcaagatccggcacaatatcgaggatggctccgtgcagctggccgaccactaccagcagaacacaccaatcggcgatggcccagtgctgctgcccgacaatcactatctgtctacccagagcgccctgtccaaggatcccaacgagaagagagaccacatggtgctgctggagttcgtgacagcagcaggaatcaccctgggaatggacgagctgtataagcgggccaagcgcggatccggagcgactaacttttccctgctgaagcaggctggcgatgtggaagagaaccctgggccaatgtccctgagcagcctgctgaaggtcgtgaccgcgtcattgtggctgggaccgggcattgcccagaagatcacccagacccagccggggatgtttgtgcaagaaaaggaagccgttaccctcgactgcacttacgacacctccgacccgtcatacggactgttctggtacaagcaacccagcagcggagaaatgatcttcctgatctaccaagggtcctacgaccagcagaatgctaccgaaggtcgctacagcctgaatttccagaaggcccgcaagagcgccaacctcgtgatttctgcctcccaactcggcgattccgcaatgtacttctgtgcgatgcggggtggcggctccggcggcagctacatccccaccttcggtcggggcacctcactgattgtgcacccatacatccagaatccggatcctgcggtctatcaattaagggactccaagtcttccgataaatccgtgtgtctctttacagacttcgactcgcaaaccaacgtgtcccagtcaaaggactcggatgtgtacatcaccgacaagactgtgctggacatgcggtcgatggacttcaagtccaacagcgcggtggcctggtccaacaagagcgacttcgcctgtgcgaacgccttcaacaactccatcattcccgaggacaccttcttcccatcccctgagtcctcctgcgacgtgaagctcgtggagaagtcgttcgagactgataccaacctgaactttcaaaacctgagcgtgatagggttcaggatcctgttactcaaagtcgccggtttcaacctcctgatgaccctgagactttggtcaagt

SEQ ID NO: 43 - protein, 765 aa, Synthetic construct
MGVPTQLLGLLLLWITDAICEIVMTQSPATLSVSPGERATLSCRASQSVSSNLAWYQQKPGQAPRLLIYGTSTRATGIPARFSGSGSGTEFTLTISSLQSEDFAVYYCQQYNNWPLTFGGGTKVEIKGGGGSGGGGSGGGGSEVQLVESGGGLVQPGRSLRLSCVASGFTFNDYAMHWVRQAPGKGLEWVSVISWNSDSIGYADSVKGRFTISRDNAKNSLYLQMHSLRAEDTALYYCAKDNHYGSGSYYYYQYGMDVWGQGTTVTVSSGGGGSTTTKPVLRTPSPVHPTGTSQPQRPEDCRPRGSVKGTGLDFACDIYIWAPLAGICVALLLSLIITLICYHRSRKWIRKKFPHIFKQPFKKTTGAAQEEDACSCRCPQEEEGGGGGYELRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRGSGATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 44 - protein, 107 aa, Synthetic construct
EIVMTQSPATLSVSPGERATLSCRASQSVSSNLAWYQQKPGQAPRLLIYGTSTRATGIPARFSGSGSGTEFTLTISSLQSEDFAVYYCQQYNNWPLTFGGGTKVEIK

SEQ ID NO: 45 - protein, 127 aa, Synthetic construct
EVQLVESGGGLVQPGRSLRLSCVASGFTFNDYAMHWVRQAPGKGLEWVSVISWNSDSIGYADSVKGRFTISRDNAKNSLYLQMHSLRAEDTALYYCAKDNHYGSGSYYYYQYGMDVWGQGTTVTVSS

SEQ ID NO: 46 - protein, 766 aa, synthetic construct
MGVPTQLLGLLLLWITDAICDIQMTQSPSSLSASVGDRVTITCRASQSISSYLNWYQQKPGKAPKLLIYAVSILQSGVPSRFSGSGSGTDFTLTINSLQPEDFATYSCQQTYSTPPITFGQGTRLEIKGGGGSGGGGSGGGGSEVQLLESGGGLVQPGGSLRLSCAASGFTFSSYAMTWVRQAPGMGLEWVSVISGSGSETYYADSVKGRFTISRDNSKNTLYLQMNSLRAEDTAVYYCVKDSSYRSSSRAYYYYGMDVWGLGTTVTVSSGGGGSTTTKPVLRTPSPVHPTGTSQPQRPEDCRPRGSVKGTGLDFACDIYIWAPLAGICVALLLSLIITLICYHRSRKWIRKKFPHIFKQPFKKTTGAAQEEDACSCRCPQEEEGGGGGYELRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRGSGATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 47 - protein, 108 aa, synthetic construct
DIQMTQSPSSLSASVGDRVTITCRASQSISSYLNWYQQKPGKAPKLLIYAVSILQSGVPSRFSGSGSGTDFTLTINSLQPEDFATYSCQQTYSTPPITFGQGTRLEIK

SEQ ID NO: 48 - protein, 127 aa, synthetic construct
EVQLLESGGGLVQPGGSLRLSCAASGFTFSSYAMTWVRQAPGMGLEWVSVISGSGSETYYADSVKGRFTISRDNSKNTLYLQMNSLRAEDTAVYYCVKDSSYRSSSRAYYYYGMDVWGLGTTVTVSS

SEQ ID NO: 49 - protein, 20 aa, synthetic construct
MGVPTQLLGLLLLWITDAIC

SEQ ID NO: 50 - protein, 15 aa, synthetic construct
GGGGSGGGGSGGGGS

SEQ ID NO: 51 - protein, 5 aa, synthetic construct
GGGGS

SEQ ID NO: 52 - protein, 72 aa, synthetic construct
TTTKPVLRTPSPVHPTGTSQPQRPEDCRPRGSVKGTGLDFACDIYIWAPLAGICVALLLSLIITLICYHRSR

SEQ ID NO: 53 - protein, 45 aa, synthetic construct
KWIRKKFPHIFKQPFKKTTGAAQEEDACSCRCPQEEEGGGGGYEL

SEQ ID NO: 54 - protein, 113 aa, synthetic construct
RAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 55 - protein, 22 aa, synthetic construct
GSGATNFSLLKQAGDVEENPGP

SEQ ID NO: 56 - protein, 239 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 57 - protein, 748 aa, synthetic construct
MGVPTQLLGLLLLWITDAICEIVLTQSPDTLSLSPGERATLSCRASQSLSSNYLAWYRQKPGQAPRLLIYGISSRATGIPDRFSGSGSGTDFTLTISRLEPEDFAVYYCQQYGSSPWTFGQGTKVEIKGGGGSGGGGSGGGGSQVQLVESGGGVVQPGRSLRLSCVASGFTFSNYGIHWVRQAPGKGLEWVAVISDDGSFKFYADSVKGRFTISRDNSKNTLYLQMNSLRVEDSAVYHCAKWQHNWNDGGFDYWGQGTLVTVSSTTTKPVLRTPSPVHPTGTSQPQRPEDCRPRGSVKGTGLDFACDIYIWAPLAGICVALLLSLIITLICYHRSRKRGRKKLLYIFKQPFMRPVQTTQEEDGCSCRFPEEEEGGCELRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRGSGATNFSLLKQAGDVEENPGPMVGEDSVLITENMHMKLYMEGTVNDHHFKCTSEGEGKPYEGTQTMKIKVVEGGPLPFAFDILATSFMYGSKTFINHTQGIPDFFKQSFPEGFTWERITTYEDGGVLTATQDTSLQNGCLIYNVKINGVNFPSNGPVMQKKTLGWEASTEMLYPADSGLRGHAQMALKLVGGGYLHCSLKTTYRSKKPAKNLKMPGFYFVDRRLERIKEADKETYVEQHEMAVARYCDLPSKLGHS

SEQ ID NO: 58 - protein, 108 aa, synthetic construct
EIVLTQSPDTLSLSPGERATLSCRASQSLSSNYLAWYRQKPGQAPRLLIYGISSRATGIPDRFSGSGSGTDFTLTISRLEPEDFAVYYCQQYGSSPWTFGQGTKVEIK

SEQ ID NO: 59 - protein, 121 aa, synthetic construct
QVQLVESGGGVVQPGRSLRLSCVASGFTFSNYGIHWVRQAPGKGLEWVAVISDDGSFKFYADSVKGRFTISRDNSKNTLYLQMNSLRVEDSAVYHCAKWQHNWNDGGFDYWGQGTLVTVSS

SEQ ID NO: 60 - protein, 42 aa, synthetic construct
KRGRKKLLYIFKQPFMRPVQTTQEEDGCSCRFPEEEEGGCEL

SEQ ID NO: 61 - protein, 235 aa, synthetic construct
MVGEDSVLITENMHMKLYMEGTVNDHHFKCTSEGEGKPYEGTQTMKIKVVEGGPLPFAFDILATSFMYGSKTFINHTQGIPDFFKQSFPEGFTWERITTYEDGGVLTATQDTSLQNGCLIYNVKINGVNFPSNGPVMQKKTLGWEASTEMLYPADSGLRGHAQMALKLVGGGYLHCSLKTTYRSKKPAKNLKMPGFYFVDRRLERIKEADKETYVEQHEMAVARYCDLPSKLGHS

SEQ ID NO: 62 - protein, 104 aa, synthetic construct
IEFMYPPPYLDNERSNGTIIHIKEKHLCHTQSSPKLFWALVVVAGVLFCYGLLVTVALCVIWTNSRRNRGGQSDYMNMTPRRPGLTRKPYQPYAPARDFAAYRP

(19) United States
(12) Patent Application Publication
(10) Pub. No.: US 2024/0148867 A1
(43) Pub. Date: May 9, 2024

(54) METHODS OF TREATING CANCER WITH A COMBINATION OF ADOPTIVE CELL THERAPY AND A TARGETED IMMUNOCYTOKINE

(71) Applicant: Regeneron Pharmaceuticals, Inc., Tarrytown, NY (US)

(72) Inventors: David DiLillo, New York, NY (US); Jiaxi Wu, Pleasantville, NY (US)

(21) Appl. No.: 18/497,352

(22) Filed: Oct. 30, 2023

Related U.S. Application Data
(60) Provisional application No. 63/381,590, filed on Oct. 31, 2022.

(51) Int. Cl. A61K 39/00

(52) U.S. Cl. CPC A61K 39/4611; A61K 39/4631; A61K 39/4632; A61K 39/464424; A61K 39/46447; A61K 39/464486; A61K 2239/39

(57) ABSTRACT

The present disclosure relates to methods of increasing the efficacy of adoptive cell therapy (ACT) and methods of treating cancer, wherein the methods include administering to a subject with cancer in need thereof a combination therapy comprising a therapeutically effective amount of an ACT (e.g., an immune cell comprising a modified T cell receptor (TCR) against a tumor-associated antigen (TAA), or a chimeric antigen receptor (CAR) against a TAA) and a therapeutically effective amount of a targeted immunocytokine (e.g., a fusion protein comprising an IL2 moiety and an immunoglobulin antigen-binding domain that binds to PD1). The combination therapy demonstrates increased anti-tumor efficacy, increased duration of tumor control and/or increased overall survival, as compared to a subject administered the ACT as monotherapy or the ACT in combination with a non-targeted immunocytokine.

### SEQUENCE LISTING

The sequence listing of the present application is submitted electronically as an ST.26 formatted xml file with a file name “11195_SeqList-179227-03002”, creation date of Oct. 24, 2023, and a size of 65,705 bytes. This sequence listing submitted is part of the specification and is hereby incorporated by reference in its entirety.

### FIELD

The present disclosure relates generally to a combination therapy that includes adoptive cell therapy and a targeted immunocytokine for treating cancer.

### BACKGROUND

There are various immunotherapy strategies, including the use of adoptive cell therapy that uses a subject's own immune cells (or a donor's immune cells) to treat diseases such as cancer. In general, adoptive cell therapy involves the transfer of genetically modified T lymphocytes into the subject. Some examples of adoptive cell therapy include the use of an engineered chimeric antigen receptor (CAR) or T cell receptor (TCR). In general, a CAR comprises a single chain fragment variable region of an antibody or a binding domain specific for a tumor associated antigen (TAA) coupled via a hinge and transmembrane regions to cytoplasmic domains of T cell signaling molecules. The most common lymphocyte activation moieties include a T cell costimulatory domain in tandem with a T cell effector function triggering moiety. CAR-mediated adoptive cell therapy allows CAR-grafted T cells to directly recognize and attack the TAAs on target tumor cells.

Adoptive cell therapy using TCRs involves engineering T cells to express a specific TCR, which is a heterodimer having two subunits. Each subunit contains a constant region that anchors the receptor to the cell membrane and a hypervariable region that performs antigen recognition. TCRs can recognize tumor specific proteins on the inside and outside of cells. With TCR therapy, T cells may be harvested from a subject's or donor's blood, and then genetically modified to express a newly engineered TCR that can then be administered to the subject to target the subject's cancer. TCRs have been reported to mediate cell killing, increase B cell proliferation, and limit the development and severity of cancer.

Due in part to the inherent complexity and patient-to-patient variability of live cell culture, adoptive cell therapy agents have tended to provide limited success with variable clinical activity. Thus, there is a need to improve anti-tumor activities of adoptive cell therapy.

Immunocytokines are antibody-cytokine conjugates with the potential to preferentially localize on tumor lesions and provide anti-tumor activity at the site of disease. The cytokine interleukin 2 (IL-2 or IL2) is a pluripotent cytokine produced primarily by activated T cells. It stimulates the proliferation and differentiation of T cells, induces the generation of cytotoxic T lymphocytes (CTLs) and the differentiation of peripheral blood lymphocytes to cytotoxic cells and lymphokine-activated killer (LAK) cells, promotes cytokine and cytolytic molecule expression by T cells, facilitates the proliferation and differentiation of B-cells and the synthesis of immunoglobulin by B-cells, and stimulates the generation, proliferation, and activation of natural killer (NK) cells.

IL2 is involved in the maintenance of peripheral CD4+ CD25+ regulatory T (Treg) cells, which are also known as suppressor T cells. They suppress effector T cells from destroying their (self-)target, either through cell-cell contact by inhibiting T cell help and activation or through release of immunosuppressive cytokines such as IL-10 or TGFβ. Depletion of Treg cells was shown to enhance IL2-induced anti-tumor immunity. However, IL2 is not optimal for inhibiting tumor growth due to its pleiotropic effects. The use of IL2 as an antineoplastic agent has also been limited by serious toxicities that accompany the doses necessary to elicit adequate tumor response.

Given the foregoing, there is a need for new cancer treatments with improved therapeutic efficacy and safety profiles.

### SUMMARY

The disclosed technology addresses one or more of the foregoing needs. In one aspect, the disclosed technology relates to a method for increasing the efficacy of adoptive cell therapy (ACT), comprising: (a) selecting a subject with cancer; and (b) administering to the subject a therapeutically effective amount of an ACT in combination with a therapeutically effective amount of a targeted immunocytokine, wherein administration of the combination leads to increased efficacy and duration of anti-tumor response, as compared to a subject treated with the ACT as monotherapy.

In another aspect, the disclosed technology relates to a method for treating cancer, comprising administering to a subject in need thereof a therapeutically effective amount of an adoptive cell therapy (ACT) in combination with a therapeutically effective amount of a targeted immunocytokine, wherein administration of the combination leads to increased efficacy and duration of anti-tumor response, as compared to a subject treated with the ACT as monotherapy.

Various embodiments of either or both aspects of the disclosed methods are described herein.

In some embodiments, the ACT comprises an immune cell selected from a T cell, a tumor-infiltrating lymphocyte, and a natural killer (NK) cell. In some embodiments, the immune cell comprises a modified TCR against a tumor-associated antigen (TAA), or a chimeric antigen receptor (CAR) against a TAA. In some embodiments, the TAA is selected from AFP, ALK, BAGE proteins, BCMA, BIRC5 (survivin), BIRC7, β-catenin, brc-abl, BRCA1, BORIS, CA9, carbonic anhydrase IX, caspase-8, CALR, CCR5, CD19, CD20 (MS4A1), CD22, CD30, CD40, CDK4, CEA, CTLA4, cyclin-B1, CYP1B1, EGFR, EGFRvIII, ErbB2/Her2, ErbB3, ErbB4, ETV6-AML, EpCAM, EphA2, Fra-1, FOLR1, GAGE proteins, GD2, GD3, GloboH, glypican-3, GM3, gp100, Her2, HLA/B-raf, HLA/k-ras, HLA/MAGE-A3, hTERT, LMP2, MAGE proteins (e.g., MAGE-1, -2, -3, -4, -6, and -12), MART-1, mesothelin, ML-IAP, Muc1, Muc2, Muc3, Muc4, Muc5, Muc16 (CA-125), MUM1, NA17, NY-BR1, NY-BR62, NY-BR85, NY-ESO1, OX40, p15, p53, PAP, PAX3, PAX5, PCTA-1, PLAC1, PRLR, PRAME, PSMA (FOLH1), RAGE proteins, Ras, RGS5, Rho, SART-1, SART-3, STEAP1, STEAP2, TAG-72, TGF-β, TMPRSS2, Thompson-nouvelle antigen (Tn), TRP-1, TRP-2, tyrosinase, and uroplakin-3.

In some embodiments, the targeted immunocytokine is a fusion protein comprising (a) an immunoglobulin antigen-binding domain of a checkpoint inhibitor and (b) an IL2 moiety. In some embodiments, the IL2 moiety comprises (i) IL2 receptor alpha (IL2Ra) or a fragment thereof; and (ii) IL2 or a fragment thereof. In some embodiments, the checkpoint inhibitor is an inhibitor of PD1, PD-L1, PD-L2, LAG-3, CTLA-4, TIM3, A2aR, B7H1, BTLA, CD160, LAIR1, TIGHT, VISTA, or VTCN1. In some embodiments, the checkpoint inhibitor is an inhibitor of PD-1.

In some embodiments, the antigen-binding domain comprises a heavy chain variable region (HCVR) comprising an amino acid sequence selected from SEQ ID NOs: 1, 11, and 20; and a light chain variable region (LCVR) comprising an amino acid sequence selected from SEQ ID NOs: 5 and 15. In some embodiments, the antigen-binding domain comprises three heavy chain complementarity determining regions (CDRs) (HCDR1, HCDR2, and HCDR3) and three light chain CDRs (LCDR1, LCDR2, and LCDR3) wherein HCDR1, HCDR2, HCDR3, LCDR1, LCDR2, and LCDR3 comprise the amino acid sequences selected from: (a) SEQ ID NOs: 2, 3, 4, 6, 7, and 8, respectively; (b) SEQ ID NOs: 12, 13, 14, 16, 7, and 17, respectively; and (c) SEQ ID NOs: 21, 22, 23, 6, 7, and 8, respectively. In some embodiments, the antigen-binding domain comprises a HCVR/LCVR amino acid sequence pair selected from SEQ ID NOs: 1/5, 11/15, and 20/5.

In some embodiments, the fusion protein comprises a heavy chain comprising a HCVR and a heavy chain constant region of IgG1 isotype. In some embodiments, the fusion protein comprises a heavy chain comprising a HCVR and a heavy chain constant region of IgG4 isotype. In some embodiments, the fusion protein comprises a heavy chain constant region comprising the amino acid sequence of SEQ ID NO: 26. In some embodiments, the fusion protein comprises a heavy chain comprising an amino acid sequence selected from SEQ ID NOs: 9, 18, and 24; and a light chain comprising an amino acid sequence selected from SEQ ID NOs: 10, 19, and 25. In some embodiments, the fusion protein comprises: (a) a heavy chain comprising the amino acid sequence of SEQ ID NO: 24, and a light chain comprising the amino acid sequence of SEQ ID NO: 25; (b) a heavy chain comprising the amino acid sequence of SEQ ID NO: 9, and a light chain comprising the amino acid sequence of SEQ ID NO: 10; or (c) a heavy chain comprising the amino acid sequence of SEQ ID NO: 18, and a light chain comprising the amino acid sequence of SEQ ID NO: 19.

In some embodiments, the antigen-binding domain comprises a heavy chain and the IL2 moiety is attached to the C-terminus of the heavy chain via a linker comprising the amino acid sequence of SEQ ID NO: 30 or 31. In some embodiments, the IL2 moiety comprises the amino acid sequence of SEQ ID NO: 27. In some embodiments, the IL2 moiety comprises wild type IL2. In some embodiments, the IL2 comprises an amino acid sequence of SEQ ID NO: 29. In some embodiments, wherein the IL2 moiety comprises the IL2 or fragment thereof connected via a linker to the C-terminus of the IL2Ra or fragment thereof. In some embodiments, the IL2Ra or fragment thereof comprises an amino acid sequence of SEQ ID NO: 28. In some embodiments, wherein the fusion protein is a dimeric fusion protein that dimerizes through the heavy chain constant region of each monomer.

In some embodiments, the targeted immunocytokine comprises a PD-1 targeting moiety and an IL2 moiety. In some embodiments, the PD-1 targeting moiety comprises an immunoglobulin antigen-binding domain that binds specifically to PD-1. In some embodiments, the antigen-binding domain comprises: (a) a HCVR comprising the amino acid sequence of SEQ ID NO: 20, and a LCVR comprising the amino acid sequence of SEQ ID NO: 5; (b) a HCVR comprising the amino acid sequence of SEQ ID NO: 1, and a LCVR comprising the amino acid sequence of SEQ ID NO: 5; or (c) a HCVR comprising the amino acid sequence of SEQ ID NO: 11; and a LCVR comprising the amino acid sequence of SEQ ID NO: 15. In some embodiments, the IL2 moiety comprises (i) IL2Ra or a fragment thereof; and (ii) IL2 or a fragment thereof. In some embodiments, the IL2 moiety comprises the amino acid sequence of SEQ ID NO: 27. In some embodiments, the targeted immunocytokine is REGN10597.

In some embodiments, the cancer is selected from adrenal gland tumors, biliary cancer, bladder cancer, brain cancer, breast cancer, carcinoma, central or peripheral nervous system tissue cancer, cervical cancer, colon cancer, endocrine or neuroendocrine cancer or hematopoietic cancer, esophageal cancer, fibroma, gastrointestinal cancer, glioma, head and neck cancer, Li-Fraumeni tumors, liver cancer, lung cancer, lymphoma, melanoma, meningioma, neuroendocrine type I or type II tumors, multiple myeloma, myelodysplastic syndromes, myeloproliferative diseases, nasopharyngeal cancer, oral cancer, oropharyngeal cancer, osteogenic sarcoma tumors, ovarian cancer, pancreatic cancer, pancreatic islet cell cancer, parathyroid cancer, pheochromocytoma, pituitary tumor, prostate cancer, rectal cancer, renal cancer, respiratory cancer, sarcoma, skin cancer, stomach cancer, testicular cancer, thyroid cancer, tracheal cancer, urogenital cancer, and uterine cancer.

In some embodiments, administration of the combination produces a therapeutic effect selected from one or more of: delay in tumor growth, reduction in tumor cell number, tumor regression, increase in survival, partial response, and complete response. In some embodiments, the therapeutically effective amount of the ACT comprises 1×10⁶ or more immune cells. In some embodiments, the therapeutically effective amount of the targeted immunocytokine is 0.005 mg/kg to 10 mg/kg of the subject's body weight. In some embodiments, the targeted immunocytokine is administered intravascularly, subcutaneously, intraperitoneally, or intratumorally. In some embodiments, the ACT is administered via intravenous infusion.

In some embodiments, the ACT is administered before or after administration of the targeted immunocytokine. In some embodiments, the ACT is administered concurrently with administration of the targeted immunocytokine. In some embodiments, the targeted immunocytokine and/or the ACT is administered in one or more doses to the subject.

In some embodiments, the method includes administering an additional therapeutic agent or therapy to the subject. In some embodiments, the additional therapeutic agent or therapy is selected from radiation, surgery, a chemotherapeutic agent, a cancer vaccine, a B7-H3 inhibitor, a B7-H4 inhibitor, a lymphocyte activation gene 3 (LAG3) inhibitor, a T cell immunoglobulin and mucin-domain containing-3 (TIM3) inhibitor, a galectin 9 (GAL9) inhibitor, a V-domain immunoglobulin (Ig)-containing suppressor of T cell activation (VISTA) inhibitor, a Killer-Cell Immunoglobulin-Like Receptor (KIR) inhibitor, a B and T lymphocyte attenuator (BTLA) inhibitor, a T cell immunoreceptor with Ig and ITIM domains (TIGIT) inhibitor, a CD47 inhibitor, an indoleamine-2,3-dioxygenase (IDO) inhibitor, a vascular endothelial growth factor (VEGF) antagonist, an angiopoietin-2 (Ang2) inhibitor, a transforming growth factor beta (TGFβ) inhibitor, an epidermal growth factor receptor (EGFR) inhibitor, an antibody to a tumor-specific antigen, Bacillus Calmette-Guerin vaccine, granulocyte-macrophage colony-stimulating factor (GM-CSF), a cytotoxin, an interleukin 6 receptor (IL-6R) inhibitor, an interleukin 4 receptor (IL-4R) inhibitor, an IL-10 inhibitor, IL-2, IL-7, IL-12, IL-21, IL-15, an antibody-drug conjugate, an anti-inflammatory drug, and combinations thereof.

In another aspect, the disclosed technology relates to an immune cell comprising a modified T cell receptor or chimeric antigen receptor that binds specifically to a tumor-associated antigen for use in a method of treating or inhibiting the growth of a tumor in combination with a targeted immunocytokine comprising: (i) an antigen-binding moiety that binds specifically to human PD-1 and (ii) an IL2 moiety, wherein the method comprises administering to a subject in need thereof a therapeutically effective amount of the immune cells and a therapeutically effective amount of the targeted immunocytokine.

### BRIEF DESCRIPTION OF THE DRAWINGS

FIG. 1 is a diagram showing an example MAGE-A4 TCR-T lentiviral construct for generating MAGE-A4₂₃₀₋₂₃₉ tetramer-positive TCR-T cells, as described in Example 2.

FIG. 2 is a graph showing the results of an in vivo study, as measured by tumor volume (mm³) of A375 tumors in mice receiving irrelevant control TCR-T cells, control TCR-T+REGN9903, control TCR-T+REGN10597, 4×10⁶ MAGE-A4 TCR-T, 4×10⁶ MAGE-A4 TCR-T+REGN9903, or 4×10⁶ MAGE-A4 TCR-T+REGN10597, as described in Example 2.

FIG. 3 is a graph showing the results of an in vivo study, as measured by tumor volume (mm³) of A375 tumors in mice receiving 2×10⁶ MAGE-A4 TCR-T, 2×10⁶ MAGE-A4 TCR-T+REGN9903, or 2×10⁶ MAGE-A4 TCR-T+REGN10597, as described in Example 2.

FIG. 4 is a graph showing the results of an in vivo study, as measured by tumor volume (mm³) of A375 tumors in mice receiving 1×10⁶ MAGE-A4 TCR-T, 1×10⁶ MAGE-A4 TCR-T+REGN9903, or 1×10⁶ MAGE-A4 TCR-T+REGN10597, as described in Example 2.

FIG. 5 is a graph showing the results of an in vivo study, as measured by tumor volume (mm³) of A375 tumors in mice receiving 4×10⁶ MAGE-A4 TCR-T, as described in Example 2.

FIG. 6 is a graph showing the results of an in vivo study, as measured by tumor volume (mm³) of A375 tumors in mice receiving 4×10⁶ MAGE-A4 TCR-T+REGN9903, as described in Example 2.

FIG. 7 is a graph showing the results of an in vivo study, as measured by tumor volume (mm³) of A375 tumors in mice receiving 4×10⁶ MAGE-A4 TCR-T+REGN10597, as described in Example 2.

FIG. 8 is a graph showing the results of an in vivo study, as measured by tumor volume (mm³) of A375 tumors in mice receiving 2×10⁶ MAGE-A4 TCR-T, as described in Example 2.

FIG. 9 is a graph showing the results of an in vivo study, as measured by tumor volume (mm³) of A375 tumors in mice receiving 2×10⁶ MAGE-A4 TCR-T+REGN9903, as described in Example 2.

FIG. 10 is a graph showing the results of an in vivo study, as measured by tumor volume (mm³) of A375 tumors in mice receiving 2×10⁶ MAGE-A4 TCR-T+REGN10597, as described in Example 2.

FIG. 11 is a graph showing the results of an in vivo study, as measured by tumor volume (mm³) of A375 tumors in mice receiving 1×10⁶ MAGE-A4 TCR-T, as described in Example 2.

FIG. 12 is a graph showing the results of an in vivo study, as measured by tumor volume (mm³) of A375 tumors in mice receiving 1×10⁶ MAGE-A4 TCR-T+REGN9903, as described in Example 2.

FIG. 13 is a graph showing the results of an in vivo study, as measured by tumor volume (mm³) of A375 tumors in mice receiving 1×10⁶ MAGE-A4 TCR-T+REGN10597, as described in Example 2.

FIG. 14 is a graph showing the results of an in vivo study, as measured by percent survival of mice receiving 4×10⁶ MAGE-A4 TCR-T, 4×10⁶ MAGE-A4 TCR-T+REGN9903, or 4×10⁶ MAGE-A4 TCR-T+REGN10597, as described in Example 2.

FIG. 15 is a graph showing the results of an in vivo study, as measured by percent survival of mice receiving 2×10⁶ MAGE-A4 TCR-T, 2×10⁶ MAGE-A4 TCR-T+REGN9903, or 2×10⁶ MAGE-A4 TCR-T+REGN10597, as described in Example 2.

FIG. 16 is a graph showing the results of an in vivo study, as measured by percent survival of mice receiving 1×10⁶ MAGE-A4 TCR-T, 1×10⁶ MAGE-A4 TCR-T+REGN9903, or 1×10⁶ MAGE-A4 TCR-T+REGN10597, as described in Example 2.

FIGS. 17A-17C are a set of diagrams showing example CAR constructs: FIG. 17A is anti-huCD20 CAR-T with CD3z and 4-1BB signaling domains (CD20/BBz CAR-T); FIG. 17B is anti-huCD20 CAR-T with CD3z and CD28 signaling domains (CD20/28z CAR-T); and FIG. 17C is Control CAR-T with CD3z and 4-1BB signaling domains (CTRL/BBz CAR-T), as described in Example 3.

FIG. 18 is a graph showing the results of an in vivo study, as measured by tumor volume (mm³) of tumors in C57BL/6 mice receiving 0.5×10⁶ CTRL/BBz CAR-T+0.2 mg/kg REGN9903, 0.5×10⁶ CD20/BBz CAR-T+0.2 mg/kg REGN9903, 0.5×10⁶ CTRL/BBz CAR-T+0.2 mg/kg REGN10597, 0.5×10⁶ CD20/BBZ CAR-T+0.2 mg/kg REGN10597, or 0.5×10⁶ CD20/BBZ CAR-T+0.5 mg/kg REGN10597, as described in Example 3.

FIG. 19 is a graph showing the results of an in vivo study, as measured by tumor volume (mm³) of tumors in C57BL/6 mice receiving 0.5×10⁶ CTRL/BBz CAR-T+0.2 mg/kg REGN9903, 0.5×10⁶ CD20/CD28Z CAR-T+0.2 mg/kg REGN9903, 0.5×10⁶ CTRL/BBz CAR-T+0.2 mg/kg REGN10597, 0.5×10⁶ CD20/28Z CAR-T+0.2 mg/kg REGN 10597, or 0.5×10⁶ CD20/28z CAR-T+0.5 mg/kg REGN 10597, as described in Example 3.

FIG. 20 is a graph showing the results of an in vivo study, as measured by tumor volume (mm³) of tumors in C57BL/6 mice receiving 0.5×10⁶ CTRL/BBz CAR-T+0.2 mg/kg REGN9903, as described in Example 3.

FIG. 21 is a graph showing the results of an in vivo study, as measured by tumor volume (mm³) of tumors in C57BL/6 mice receiving 0.5×10⁶ CTRL/BBz CAR-T+0.2 mg/kg REGN10597, as described in Example 3.

FIG. 22 is a graph showing the results of an in vivo study, as measured by tumor volume (mm³) of tumors in C57BL/6 mice receiving 0.5×10⁶ CD20/BBZ CAR-T+0.2 mg/kg REGN9903, as described in Example 3.

FIG. 23 is a graph showing the results of an in vivo study, as measured by tumor volume (mm³) of tumors in C57BL/6 mice receiving 0.5×10⁶ CD20/BBZ CAR-T+0.2 mg/kg REGN10597, as described in Example 3.

FIG. 24 is a graph showing the results of an in vivo study, as measured by tumor volume (mm³) of tumors in C57BL/6 mice receiving 0.5×10⁶ CD20/BBZ CAR-T+0.5 mg/kg REGN10597, as described in Example 3.

FIG. 25 is a graph showing the results of an in vivo study, as measured by tumor volume (mm³) of tumors in C57BL/6 mice receiving 0.5×10⁶ CD20/CD28Z CAR-T+0.2 mg/kg REGN9903, as described in Example 3.

FIG. 26 is a graph showing the results of an in vivo study, as measured by tumor volume (mm³) of tumors in C57BL/6 mice receiving 0.5×10⁶ CD20/28Z CAR-T+0.2 mg/kg REGN10597, as described in Example 3.

FIG. 27 is a graph showing the results of an in vivo study, as measured by tumor volume (mm³) of tumors in C57BL/6 mice receiving 0.5×10⁶ CD20/28Z CAR-T+0.5 mg/kg REGN10597, as described in Example 3.

FIG. 28 is a pair of graphs showing frequency and absolute number of peripheral blood B220⁺ B cells at Day 7 in lymphodepleted mice administered the indicated combination therapies, as described in Example 4.

FIG. 29 is a pair of graphs showing frequency and absolute number of peripheral blood GFP⁺ CAR T cells at Day 7 in lymphodepleted mice administered the indicated combination therapies, as described in Example 4.

FIG. 30 is a pair of graphs showing frequency and absolute number of peripheral blood B220⁺ B cells at Day 7 in non-lymphodepleted mice administered the indicated combination therapies, as described in Example 4.

FIG. 31 is a pair of graphs showing frequency and absolute number of peripheral blood GFP⁺ CAR T cells Day 7 in non-lymphodepleted mice administered the indicated combination therapies, as described in Example 4.

FIG. 32 is a pair of graphs showing frequency and absolute number of peripheral blood B220⁺ B cells at Day 21 in lymphodepleted mice administered the indicated combination therapies, as described in Example 4.

FIG. 33 is a pair of graphs showing frequency and absolute number of peripheral blood GFP⁺ CAR T cells at Day 21 in lymphodepleted mice administered the indicated combination therapies, as described in Example 4.

FIG. 34 is a pair of graphs showing frequency and absolute number of peripheral blood B220+B cells at Day 21 in non-lymphodepleted mice administered the indicated combination therapies, as described in Example 4.

FIG. 35 is a pair of graphs showing frequency and absolute number of peripheral blood GFP⁺ CAR T cells at Day 21 in non-lymphodepleted mice administered the indicated combination therapies, as described in Example 4.

FIG. 36 is a graph showing average tumor volume in mice administered the indicated combination therapies, as described in Example 5.

FIGS. 37A-D relate to Example 6. FIG. 37A is a graph showing expression of PD-1 on anti-huMUC16 or control CAR+ T cells after coculture with indicated tumor cell lines in vitro.

FIG. 37B is a schematic of the in vivo study. FIG. 37C is a graph showing average tumor growth (mean+SD) monitored over time, with statistical analyses performed using two-way ANOVA with Bonferroni's multiple comparisons tests (**P≤0.01, ***P≤0.001, ****P≤0.0001). FIG. 37D is a collection of individual tumor growth curves, wherein the data are representative of results from experiments performed with two different syngeneic tumor models.

### DETAILED DESCRIPTION

The disclosed technology is based, at least in part, on an unexpected discovery that a targeted immunocytokine augments in vivo anti-tumor activities of immune cells (e.g., T cells) comprising a modified TCR or a CAR. Cell therapies for treating cancer (referred to herein as “adoptive cell therapy,” ACT, or adoptive immunotherapy) include immune cells (e.g., T cells) which are modified with a TCR or a CAR wherein the TCR or CAR is targeted to a TAA. Such cell therapies show modest and non-durable tumor control. IL2 is administered for cell proliferation and expansion; however, naked IL2 or non-targeted IL2 leads to toxicity in the subject. In contrast, without being bound to a particular theory, it is believed that when IL2 is co-administered with a moiety targeted to a checkpoint inhibitor (referred to herein as a “targeted immunocytokine”), the combination provides a targeted agent driving the proliferation, expansion and survival of the immune cells. Enhanced survival corresponds to increased duration of anti-tumor response. As described herein, administration of a targeted immunocytokine leads to increased survival and longer duration of anti-tumor activity of T cells modified with a TCR or CAR against a TAA. Non-limiting examples of such TAAs include MAGE-A4 and CD20, among others. The aforementioned co-administration leads to greater anti-tumor response (e.g., greater shrinking of tumors) and a longer duration of response in the mice. Thus, the disclosed combination therapy of a targeted immunocytokine and a TCR-modified or CAR-modified immune cell demonstrates unexpected synergistic anti-tumor efficacy in inducing potent and durable tumor control in subjects with cancer.

Methods for Treating Cancer

The present disclosure includes methods of increasing the efficacy of adoptive cell therapy (ACT), wherein the method includes administering to a subject with cancer a combination therapy comprising a therapeutically effective amount of an ACT and a therapeutically effective amount of a targeted immunocytokine. The present disclosure also includes methods of treating cancer, wherein the method includes administering to a subject in need thereof a combination therapy comprising a therapeutically effective amount of an ACT and a therapeutically effective amount of a targeted immunocytokine.

As used herein, the terms “treating,” “treat” or the like, mean to alleviate symptoms, eliminate the causation of symptoms either on a temporary or permanent basis, to delay or inhibit tumor growth, to reduce tumor cell load or tumor burden, to promote tumor regression, to cause tumor shrinkage, necrosis and/or disappearance, to prevent tumor recurrence, to prevent or inhibit metastasis, to inhibit metastatic tumor growth, and/or to increase duration of survival of the subject.

As used herein, the expression “a subject in need thereof” refers to a human or non-human mammal that exhibits one or more symptoms or indications of cancer, and/or who has been diagnosed with cancer and who needs treatment for the same. The term “subject” includes subjects with primary or metastatic tumors (advanced malignancies). In certain embodiments, the expression “a subject in need thereof” includes a subject with a tumor that is resistant to or refractory to or is inadequately controlled by prior therapy (e.g., treatment with an anti-cancer agent). The expression also includes subjects with a tumor for which conventional anti-cancer therapy is inadvisable, for example, due to toxic side effects. For example, the expression includes subjects who have received one or more cycles of chemotherapy and have experienced toxic side effects.

As used herein, the term “tumor” or “cancer” refers to a disease characterized by the uncontrolled (and often rapid) growth of aberrant cells. Cancer cells can spread locally or through the bloodstream and lymphatic system to other parts of the body. Examples of various cancers are described herein and include, but are not limited to, breast cancer, prostate cancer, ovarian cancer, cervical cancer, skin cancer, pancreatic cancer, colorectal cancer, renal cancer, liver cancer, brain cancer, adrenal gland cancer, autonomic ganglial cancer, biliary tract cancer, bone cancer, endometrial cancer, eye cancer, fallopian tube cancer, genital tract cancers, large intestinal cancer, cancer of the meninges, oesophageal cancer, peritoneal cancer, pituitary cancer, penile cancer, placental cancer, pleura cancer, salivary gland cancer, small intestinal cancer, stomach cancer, testicular cancer, thymus cancer, thyroid cancer, upper aerodigestive cancers, urinary tract cancer, vaginal cancer, vulva cancer, lymphoma, leukemia, lung cancer and the like. The terms “tumor,” “cancer” and “malignancy” are interchangeably used herein.

In certain embodiments, the disclosed methods for treating or inhibiting the growth of a tumor include, but are not limited to, treating or inhibiting the growth of anal cancer, bladder cancer, blood cancer, bone cancer, brain cancer, breast cancer, cervical cancer, colon cancer, colorectal cancer, endometrial cancer, esophageal cancer, gastric cancer, head and neck cancer, kidney cancer, liver cancer, lung cancer, myeloma, ovarian cancer, pancreatic cancer, prostate cancer, rectal cancer, salivary gland cancer, skin cancer, squamous cell carcinoma, stomach cancer, testicular cancer, and uterine cancer.

In some embodiments, the disclosed methods lead to increased efficacy and duration of anti-tumor response. Methods according to this aspect of the disclosure comprise selecting a subject with cancer and administering to the subject a therapeutically effective amount of a targeted immunocytokine in combination with a therapeutically effective amount of adoptive cell therapy. In certain embodiments, the methods provide for increased tumor inhibition, e.g., by about 20%, more than 20%, more than 30%, more than 40%, more than 50%, more than 60%, more than 70%, or more than 80% as compared to a subject treated with the ACT as monotherapy or treated with the ACT in combination with a non-targeted immunocytokine (such as a non-targeted IL2 cytokine).

In certain embodiments, the methods provide for increased duration of the anti-tumor response, e.g., by about 20%, more than 20%, more than 30%, more than 40%, more than 50%, more than 60%, more than 70% or more than 80% as compared to a subject treated with the ACT as monotherapy or treated with the ACT in combination with a non-targeted immunocytokine (such as a non-targeted IL2 cytokine). In certain embodiments, administration of the targeted immunocytokine in combination with ACT increases response and duration of response in a subject, e.g., by more than 2%, more than 3%, more than 4%, more than 5%, more than 6%, more than 7%, more than 8%, more than 9%, more than 10%, more than 20%, more than 30%, more than 40% or more than 50% more than an untreated subject or a subject treated with the ACT as monotherapy or treated with the ACT in combination with a non-targeted immunocytokine (such as a non-targeted IL2 cytokine).

In certain embodiments, the disclosed methods lead to a delay in tumor growth and development, e.g., tumor growth may be delayed by about 3 days, more than 3 days, about 7 days, more than 7 days, more than 15 days, more than 1 month, more than 3 months, more than 6 months, more than 1 year, more than 2 years, or more than 3 years as compared to an untreated subject or a subject treated with ACT monotherapy or treated with ACT in combination with a non-targeted immunocytokine (such as a non-targeted IL2 cytokine).

In certain embodiments, administration of any of the combinations disclosed herein prevents tumor recurrence and/or increases duration of survival of the subject, e.g., increases duration of survival by 1-5 days, by 5 days, by 10 days, by 15 days, more than 15 days, more than 1 month, more than 3 months, more than 6 months, more than 12 months, more than 18 months, more than 24 months, more than 36 months, or more than 48 months more than the survival of an untreated subject or a subject treated with ACT as monotherapy or treated with ACT in combination with a non-targeted immunocytokine (such as a non-targeted IL2 cytokine).

In certain embodiments, administration of the targeted immunocytokine in combination with ACT to a subject with a cancer leads to complete disappearance of all evidence of tumor cells (“complete response”). In certain embodiments, administration of the targeted immunocytokine in combination with ACT to a subject with a cancer leads to at least 30% or more decrease in tumor cells or tumor size (“partial response”). In certain embodiments, administration of the targeted immunocytokine in combination with ACT to a subject with a cancer leads to complete or partial disappearance of tumor cells/lesions including new measurable lesions. Tumor reduction can be measured by any methods known in the art, e.g., X-rays, positron emission tomography (PET), computed tomography (CT), magnetic resonance imaging (MRI), cytology, histology, or molecular genetic analyses.

In certain embodiments, administration of the targeted immunocytokine in combination with ACT to a subject with a cancer leads to improved overall response rate, as compared to an untreated subject or a subject treated with ACT monotherapy or treated with ACT in combination with a non-targeted immunocytokine (such as a non-targeted IL2 cytokine).

In certain embodiments, administering to a subject with cancer therapeutically effective amounts of the disclosed ACT and targeted immunocytokine leads to increased overall survival (OS) or progression-free survival (PFS) of the subject as compared to a subject treated with ACT as monotherapy or treated with ACT in combination with a non-targeted immunocytokine (such as a non-targeted IL2 cytokine).

In certain embodiments, the PFS is increased by at least one month, at least 2 months, at least 3 months, at least 4 months, at least 5 months, at least 6 months, at least 7 months, at least 8 months, at least 9 months, at least 10 months, at least 11 months, at least 1 year, at least 2 years, or at least 3 years as compared to a subject treated with ACT as monotherapy or treated with ACT in combination with a non-targeted immunocytokine (such as a non-targeted IL2 cytokine).

In certain embodiments, the OS is increased by at least one month, at least 2 months, at least 3 months, at least 4 months, at least 5 months, at least 6 months, at least 7 months, at least 8 months, at least 9 months, at least 10 months, at least 11 months, at least 1 year, at least 2 years, or at least 3 years as compared to a subject treated with ACT as monotherapy or treated with ACT in combination with a non-targeted immunocytokine (such as a non-targeted IL2 cytokine).

Adoptive Cell Therapy (ACT)

The disclosed methods include administration of a targeted immunocytokine in combination with ACT. As used herein, the term “adoptive cell therapy,” “ACT” or “adoptive immunotherapy” are used interchangeably and refer to the administration of a modified immune cell to a subject with cancer. An “immune cell” (also interchangeably referred to herein as an “immune effector cell”) refers to a cell that is part of a subject's immune system and helps to fight cancer in the body of a subject. Non-limiting examples of immune cells for use in the disclosed methods include T cells, tumor-infiltrating lymphocytes, and natural killer (NK) T cells. The immune cells may be autologous or heterologous to the subject undergoing therapy.

As used herein, the terms “T cell” and “T lymphocyte” are used interchangeably. T cells include thymocytes, naive T lymphocytes, immature T lymphocytes, mature T lymphocytes, resting T lymphocytes, or activated T lymphocytes. A T cell can be a T helper (Th) cell, for example, a T helper 1 (Th1) or a T helper 2 (Th2) cell. The T cell can be a helper T cell (HTL; CD4⁺ T cell) CD4⁺ T cell, a cytotoxic T cell (CTL; CD8⁺ T cell), a tumor-infiltrating cytotoxic T cell (TIL; CD8⁺ T cell), CD4+CD8⁺ T cell, or any other subset of T cells. Other illustrative populations of T cells suitable for use in particular embodiments include naive T cells and memory T cells. Also included are “natural killer T (NKT) cells” or “NKT cells,” which refer to a specialized population of T cells that express a semi-invariant ab T cell receptor, but also express a variety of molecular markers that are typically associated with NK cells, such as NK1.1. NKT cells include NK1.1+ and NK1. G, as well as CD4+, CD4, CD8+, and CD8 cells.

The TCR on NKT cells is unique in that it recognizes glycolipid antigens presented by the MHC I-like molecule CD Id. NKT cells can have either protective or deleterious effects due to their ability to produce cytokines that promote either inflammation or immune tolerance. Also included are “gamma-delta T cells (γδ T cells),” which refer to a specialized population that to a small subset of T cells possessing a distinct TCR on their surface, and unlike the majority of T cells in which the TCR is composed of two glycoprotein chains designated a- and b-TCR chains, the TCR in γδ T cells is made up of a g-chain and a d-chain. γδ T cells can play a role in immunosurveillance and immunoregulation and were found to be an important source of IL-17 and to induce robust CD8⁺ cytotoxic T cell response. Also included are “regulatory T cells” or “Tregs,” which refer to T cells that suppress an abnormal or excessive immune response and play a role in immune tolerance. Tregs are typically transcription factor Foxp3-positive CD4⁺ T cells and can also include transcription factor Foxp3-negative regulatory T cells that are IL-10-producing CD4⁺ T cells.

T cells can be obtained from a number of sources, including peripheral blood mononuclear cells, bone marrow, lymph nodes tissue, cord blood, thymus issue, tissue from a site of infection, ascites, pleural effusion, spleen tissue, and tumors. In some embodiments, T cells can be obtained from a unit of blood collected from the subject using any number of techniques known to the skilled person, such as FICOLL separation. In one embodiment, T cells from the circulating blood of an individual are obtained by apheresis. The apheresis product typically contains lymphocytes, including T cells, monocytes, granulocyte, B cells, other nucleated white blood cells, red blood cells, and platelets.

The disclosed immune effector cells, such as T cells, can be genetically modified (forming modified immune cells) following isolation using known methods, or the immune cells can be activated and expanded, or differentiated in the case of progenitors, in vitro prior to being genetically modified. In some embodiments, immune effector cells, such as T cells, are genetically modified with the TCRs or CARs described herein (e.g., transduced with a viral vector comprising a nucleic acid encoding a TCR or a CAR) and then may be activated and expanded in vitro. Techniques for activating and expanding T cells are known in the art and suitable for use with the disclosed technology. See, e.g., U.S. Pat. Nos. 6,905,874; 6,867,041; 6,797,514; WO 2012079000; US 2016/0175358. TCR-expressing or CAR-expressing immune effector cells suitable for use in the disclosed methods may be prepared according to known techniques described in the art.

For use in the disclosed methods, the immune cells may be modified with a TCR or a CAR against a TAA. In other words, non-limiting examples of ACT for use in the disclosed methods include a modified TCR against a tumor-associated antigen (TAA), or a chimeric antigen receptor (CAR) against a TAA.

The TAA may be from any cancer including, but not limited to, adrenal gland tumors, biliary cancer, bladder cancer, brain cancer, breast cancer, carcinoma, central or peripheral nervous system tissue cancer, cervical cancer, colon cancer, endocrine or neuroendocrine cancer or hematopoietic cancer, esophageal cancer, fibroma, gastrointestinal cancer, glioma, head and neck cancer, Li-Fraumeni tumors, liver cancer, lung cancer, lymphoma, melanoma, meningioma, neuroendocrine type I or type II tumors, multiple myeloma, myelodysplastic syndromes, myeloproliferative diseases, nasopharyngeal cancer, oral cancer, oropharyngeal cancer, osteogenic sarcoma tumors, ovarian cancer, pancreatic cancer, pancreatic islet cell cancer, parathyroid cancer, pheochromocytoma, pituitary tumor, prostate cancer, rectal cancer, renal cancer, respiratory cancer, sarcoma, skin cancer, stomach cancer, testicular cancer, thyroid cancer, tracheal cancer, urogenital cancer, or uterine cancer.

In certain embodiments, the TAA is selected from AFP, ALK, BAGE proteins, BCMA, BIRC5 (survivin), BIRC7, β-catenin, brc-abl, BRCA1, BORIS, CA9, carbonic anhydrase IX, caspase-8, CALR, CCR5, CD19, CD20 (MS4A1), CD22, CD30, CD40, CDK4, CEA, CTLA4, cyclin-B1, CYP1 B1, EGFR, EGFRvIII, ErbB2/Her2, ErbB3, ErbB4, ETV6-AML, EpCAM, EphA2, Fra-1, FOLR1, GAGE proteins (e.g., GAGE-1, -2), GD2, GD3, GloboH, glypican-3, GM3, gp100, Her2, HLA/B-raf, HLA/k-ras, HLA/MAGE-A3, hTERT, LMP2, MAGE proteins (e.g., MAGE-1, -2, -3, -4, -6, and -12), MART-1, mesothelin, ML-IAP, Muc1, Muc2, Muc3, Muc4, Muc5, Muc16 (CA-125), MUM1, NA17, NY-BR1, NY-BR62, NY-BR85, NY-ESO1, OX40, p15, p53, PAP, PAX3, PAX5, PCTA-1, PLAC1, PRLR, PRAME, PSMA (FOLH1), RAGE proteins, Ras, RGS5, Rho, SART-1, SART-3, STEAP1, STEAP2, TAG-72, TGF-β, TMPRSS2, Thompson-nouvelle antigen (Tn), TRP-1, TRP-2, tyrosinase, or uroplakin-3.

As used herein, a “T cell receptor” refers to an isolated TCR polypeptide that binds specifically to a TAA, or a TCR expressed on an isolated immune cell (e.g., a T cell). TCRs bind to epitopes on small antigenic determinants (for example, comprised in a tumor associated antigen) on the surface of antigen-presenting cells that are associated with a major histocompatibility complex (MHC; in mice) or human leukocyte antigen (HLA; in humans) complex. TCR also refers to an immunoglobulin superfamily member having a variable binding domain, a constant domain, a transmembrane region, and a short cytoplasmic tail (see, e.g., Janeway et al., Immunobiology: The Immune System in Health and Disease, 3rd Ed., Current Biology Publications, 1997) capable of specifically binding to an antigen peptide bound to a MHC receptor.

As used herein, the term “polypeptide” refers to any polymer preferably consisting essentially of any of the 20 natural amino acids regardless of its size. Although the term “protein” is often used in reference to relatively large proteins, and “peptide” is often used in reference to small polypeptides, use of these terms in the field often overlaps. The term “polypeptide” refers generally to proteins, polypeptides, and peptides unless otherwise noted. Peptides useful in accordance with the present disclosure will be generally between about 0.1 to 100 KD or greater up to about 1000 KD, preferably between about 0.1, 0.2, 0.5, 1, 2, 5, 10, 20, 30, and 50 KD as judged by standard molecule sizing techniques such as centrifugation or SDS-polyacrylamide gel electrophoresis.

A TCR can be found on the surface of a cell and generally is comprised of a heterodimer having α and β chains (also known as TCRα and TCRβ, respectively), or γ and δ chains (also known as TCRγ and TCRδ, respectively). Like immunoglobulins, the extracellular portion of TCR chains (e.g., α-chain, β-chain) contain two immunoglobulin regions, a variable region (e.g., TCR variable α region or Vα and TCR variable β region or Vβ; typically amino acids 1 to 116 based on Kabat numbering at the N-terminus), and one constant region (e.g., TCR constant domain α or Cα and typically amino acids 117 to 259 based on Kabat, TCR constant domain β or Cβ, typically amino acids 117 to 295 based on Kabat) adjacent to the cell membrane. Also, like immunoglobulins, the variable domains contain CDRs separated by framework regions (FRs). In some embodiments, a TCR is found on the surface of T cells (or T lymphocytes) and associates with the CD3 complex. The source of a TCR of the present disclosure may be from various animal species, such as a human, mouse, rat, rabbit or other mammal. In some embodiments, the source of a TCR of the present disclosure is a mouse genetically engineered to produce TCRs comprising human alpha and beta chains (see, e.g., WO 2016/164492).

As used herein, the terms “complementarity determining region” or “CDR” refer to the sequences of amino acids within antibody variable regions that confer antigen specificity and binding affinity. In general, there are three CDRs in each heavy chain variable region (HCDR1, HCDR2, and HCDR3) and three CDRs in each light chain variable region (LCDR1, LCDR2, and LCDR3). Exemplary conventions that can be used to identify the boundaries of CDRs include, e.g., the Kabat definition, the Chothia definition, the ABM definition, and the IMGT definition. See, e.g., Kabat, 1991, “Sequences of Proteins of Immunological Interest,” National Institutes of Health, Bethesda, Md. (Kabat numbering scheme); Al-Lazikani et al., 1997, J. Mol. Biol. 273:927-948 (Chothia numbering scheme); Martin et al., 1989, Proc. Natl. Acad. Sci. USA 86:9268-9272 (ABM numbering scheme); and Lefranc et al., 2003, Dev. Comp. Immunol. 27:55-77 (IMGT numbering scheme). Public databases are also available for identifying CDR sequences within an antibody.

TCRα and TCRβ polypeptides (and similarly TCRγ and TCRδ polypeptides) are linked to each other via a disulfide bond. Each of the two polypeptides that make up the TCR contains an extracellular domain comprising constant and variable regions, a transmembrane domain, and a cytoplasmic tail (the transmembrane domain and the cytoplasmic tail also being a part of the constant region). The variable region of the TCR determines its antigen specificity, and similar to immunoglobulins, comprises three CDRs. The TCR is expressed on most T cells in the body and is known to be involved in recognition of MHC-restricted antigens. The TCR α chain includes a covalently linked Vα and Cα region, whereas the β chain includes a Vβ region covalently linked to a Cβ region. The Vα and Vβ regions form a pocket or cleft that can bind an antigen in the context of a major histocompatibility complex (MHC) (or HLA in humans).

The term “HLA” refers to the human leukocyte antigen (HLA) system or complex, which is a gene complex encoding the MHC proteins in humans. These cell-surface proteins are responsible for regulating the immune system in humans. HLAs corresponding to MHC class I (A, B, and C) present peptides from inside the cell. The term “HLA-A” refers to the group of human leukocyte antigens (HLA) that are coded for by the HLA-A locus. HLA-A is one of three major types of human MHC class I cell surface receptors. The receptor is a heterodimer and composed of a heavy a chain and a smaller β chain. The α chain is encoded by a variant HLA-A gene, and the β chain (β2-microglobulin) is an invariant β2 microglobulin molecule. The term “HLA-A2” (also referred to as “HLA-A2*01”) is one particular MHC class I allele group at the HLA-A locus; the α chain is encoded by the HLA-A*02 gene, and the β chain is encoded by the P2-microglobulin or B2M locus.

TCRs are detection molecules with exquisite specificity, and exhibit, like antibodies, an enormous diversity. The general structure of TCR molecules and techniques for making and using such molecules, including binding to a peptide: MHC, are described in PCT/US98/04274, PCT/US98/20263, WO 99/60120.

For example, non-human animals (e.g., rodents, e.g., mice or rats) can be genetically engineered to express a human or humanized TCR comprising a variable domain encoded by at least one human TCR variable region gene segment. See, e.g., WO 2016/164492. For example, the Veloci-T® mouse technology (Regeneron) provides a genetically modified mouse that allows for the production of fully human therapeutic TCRs against tumor and/or viral antigens, and can be used to produce TCRs suitable for use with the disclosed technology. Those of skill in the art, through standard mutagenesis techniques, in conjunction with the assays described herein, can obtain altered TCR sequences and test them for particular binding affinity and/or specificity. Useful mutagenesis techniques known in the art include, without limitation, de novo gene synthesis, oligonucleotide-directed mutagenesis, region-specific mutagenesis, linker-scanning mutagenesis, and site-directed mutagenesis by PCR.

In some embodiments, methods for generating a TCR to a TAA may include immunizing a non-human animal (e.g., a rodent, e.g., a mouse or a rat), such as a genetically engineered non-human animal that comprises in its genome an un-rearranged human TCR variable gene locus, with a specified peptide from the TAA; allowing the animal to mount an immune response to the peptide; isolating from the animal a T cell reactive to the peptide; determining a nucleic acid sequence of a human TCR variable region expressed by the T cell; cloning the human TCR variable region into a nucleotide construct comprising a nucleic acid sequence of a human TCR constant region such that the human TCR variable region is operably linked to the human TCR constant region; and expressing from the construct a human T cell receptor specific for the peptide, respectively. The steps of isolating a T cell, determining a nucleic acid sequence of a human TCR variable region expressed by the T cell, cloning the human TCR variable region into a nucleotide construct comprising a nucleic acid sequence of a human TCR constant region, and expressing a human T cell receptor are performed using standard techniques known to those of skill the art.

As used herein, an HLA presented peptide (such as an HLA-A2 presented peptide) can refer to a peptide that is bound to a HLA protein, such as an HLA protein expressed on the surface of a cell. Thus, a TCR that binds to an HLA presented peptide binds to the peptide that is bound by the HLA, and optionally also binds to the HLA itself. Interaction with the HLA can confer specificity for binding to a peptide presented by a particular HLA. In some embodiments, the TCR may bind to an isolated HLA presented peptide. In some embodiments, the TCR may bind to an HLA presented peptide on the surface of a cell.

As used herein, a “chimeric antigen receptor” or “CAR” refers to an antigen-binding protein that includes an immunoglobulin antigen-binding domain (e.g., an immunoglobulin variable domain) and a TCR constant domain or a portion thereof, which can be administered to a subject as chimeric antigen receptor T-cell (CAR-T) therapy. As used herein, a “constant domain” of a TCR polypeptide includes a membrane-proximal TCR constant domain, and may also include a TCR transmembrane domain and/or a TCR cytoplasmic tail. For example, in some embodiments, the CAR is a dimer that includes a first polypeptide comprising an immunoglobulin heavy chain variable domain linked to a TCRβ constant domain and a second polypeptide comprising an immunoglobulin light chain variable domain (e.g., a κ or λ variable domain) linked to a TCRα constant domain. In some embodiments, the CAR is a dimer that includes a first polypeptide comprising an immunoglobulin heavy chain variable domain linked to a TCRα constant domain and a second polypeptide comprising an immunoglobulin light chain variable domain (e.g., a κ or λ variable domain) linked to a TCRβ constant domain.

As used herein, a “variable domain” refers to the variable region of an alpha chain or the variable region of a beta chain that is involved directly in binding the TCR to the antigen. As used herein, the term “constant domain” refers to the constant region of the alpha chain and the constant region of the beta chain that are not involved directly in binding of a TCR to an antigen, but exhibit various effector functions.

CARs are typically artificial, constructed hybrid proteins or polypeptides containing the antigen-binding domain of an scFv or other antibody agent linked to a T cell signaling domain. In the context of this disclosure, the CAR is directed to a tumor-associated antigen. Features of the CAR include its ability to redirect T cell specificity and reactivity against selected targets in a non-MHC-restricted manner using the antigen-binding properties of monoclonal antibodies. Non-MHC-restricted antigen recognition provides CAR-expressing T cells with the ability to recognize antigens independent of antigen processing, thereby bypassing the major mechanism of tumor escape. As used in the ACT disclosed herein, immune cells can be manipulated to express the CAR in any known manner, including, for example, by transfection using RNA and DNA, both techniques being known in the art.

In some embodiments, TCR- or CAR-expressing immune effector cells are formulated by first harvesting them from their culture medium, and then washing and concentrating the cells in a medium and container system suitable for administration (a “pharmaceutically acceptable” carrier) in a treatment-effective amount. A suitable infusion medium can be any isotonic medium formulation, typically normal saline, Normosol R (Abbott) or Plasma-Lyte A (Baxter), but also 5% dextrose in water or Ringer's lactate can be utilized. The infusion medium may be supplemented with human serum albumin.

A therapeutically effective number of immune cells to be administered in the disclosed methods is typically greater than 10² cells, such as up to and including 10⁶, up to and including 10⁸, up to and including 10⁹ cells, or more than 10¹⁰ cells. The number and/or type of cells to be administered to a subject will depend upon the ultimate use for which the therapy is intended.

TCRs and CARs of the present disclosure may be recombinant, meaning that they may be created, expressed, isolated or obtained by technologies or methods known in the art as recombinant DNA technology, which include, e.g., DNA splicing and transgenic expression. Recombinant TCRs or CARs may be expressed in a non-human mammal (including transgenic non-human mammals, e.g., transgenic mice), or a cell (e.g., CHO cells) expression system or isolated from a recombinant combinatorial human antibody library.

Targeted Immunocytokines

As used herein, a “targeted immunocytokine” refers to a cytokine such as interleukin 2 (IL2) that is linked to a moiety that binds to a checkpoint inhibitor (i.e., “targets” a checkpoint inhibitor). Non-limiting examples of the checkpoint inhibitor include inhibitors of PD1, PD-L1, PD-L2, LAG-3, CTLA-4, TIM3, A2aR, B7H1, BTLA, CD160, LAIR1, TIGHT, VISTA, or VTCN1. In some embodiments, the targeted immunocytokine includes an immunoglobulin antigen-binding domain of a checkpoint inhibitor. In one preferred embodiment, the checkpoint inhibitor is an inhibitor of PD-1 (e.g., an anti-PD-1 antibody or antigen-binding fragment thereof). In certain embodiments, the targeted immunocytokine is a fusion protein that includes (i) an antigen-binding domain of a checkpoint inhibitor and (ii) an IL2 moiety.

In some embodiments, the antigen-binding domain binds specifically to human PD-1. In some embodiments, the antigen-binding domain is an antibody or antigen-binding fragment thereof.

As used herein, the term “fusion protein” means a protein comprising two or more polypeptide sequences that are joined together covalently or non-covalently. Fusion proteins encompassed by the present disclosure may include translation products of a chimeric gene construct that joins the nucleic acid sequences encoding a first polypeptide with the nucleic acid sequence encoding a second polypeptide to form a single open reading frame. Alternatively, the fusion protein may be encoded by two or more gene constructs on separate vectors that may be co-expressed in a host cell. In general, a “fusion protein” is a recombinant protein of two or more proteins joined by a peptide bond or by several peptides. In some embodiments, the fusion protein may also comprise a peptide linker between the two domains.

Fusion proteins disclosed herein may include one or more conservative modifications. A fusion protein with one or more conservative modifications may retain the desired functional properties, which can be tested using the functional assays known in the art. The term “conservative sequence modifications” refers to amino acid modifications that do not significantly affect or alter the binding characteristics of the protein containing the amino acid sequence. Such conservative modifications include amino acid substitutions, additions, and deletions. Modifications can be introduced by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include: amino acids with basic side chains (e.g., lysine, arginine, histidine); acidic side chains (e.g., aspartic acid, glutamic acid); uncharged polar side chains (e.g., glycine, asparagine, glutamine, serine, threonine, tyrosine, cysteine, tryptophan); nonpolar side chains (e.g., alanine, valine, leucine, isoleucine, proline, phenylalanine, methionine); beta-branched side chains (e.g., threonine, valine, isoleucine); and aromatic side chains (e.g., tyrosine, phenylalanine, tryptophan, histidine). includes one or more conservative modifications. The Cas protein with one or more conservative modifications may retain the desired functional properties, which can be tested using the functional assays known in the art. As used herein, the term “conservative sequence modifications” refers to amino acid modifications that do not significantly affect or alter the binding characteristics of the protein containing the amino acid sequence. Such conservative modifications include amino acid substitutions, additions, and deletions. Modifications can be introduced by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include: amino acids with basic side chains (e.g., lysine, arginine, histidine); acidic side chains (e.g., aspartic acid, glutamic acid); uncharged polar side chains (e.g., glycine, asparagine, glutamine, serine, threonine, tyrosine, cysteine, tryptophan); nonpolar side chains (e.g., alanine, valine, leucine, isoleucine, proline, phenylalanine, methionine); beta-branched side chains (e.g., threonine, valine, isoleucine); and aromatic side chains (e.g., tyrosine, phenylalanine, tryptophan, histidine).

As used herein, an “antibody” refers to an immunoglobulin molecule comprised of four polypeptide chains, two heavy (H) chains and two light (L) chains inter-connected by disulfide bonds (i.e., “full antibody molecules”), as well as a multimer thereof (e.g., IgM) or antigen-binding fragments thereof. Each heavy chain is comprised of a heavy chain variable region (“HCVR” or “VH”) and a heavy chain constant region (comprised of domains CH1, CH2, and CH3). Each light chain is comprised of a light chain variable region (“LCVR or “VL”) and a light chain constant region (CL). The VH and VL regions can be further subdivided into regions of hypervariability, termed CDRs, interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. In some embodiments, the FRs of the antibody (or antigen-binding fragment thereof) may be identical to the human germline sequences or may be naturally or artificially modified. An amino acid consensus sequence may be defined based on a side-by-side analysis of two or more CDRs. The term “antibody” also includes antigen-binding fragments of full antibody molecules.

As used herein, an “antigen” refers to any substance that causes the immune system to produce antibodies or specific cell-mediated immune responses against it. A disease-associated antigen is any substance that is associated with any disease that causes the immune system to produce antibodies or a specific cell-mediated response against it.

As used herein, the “antigen-binding fragment” of an antibody, “antigen-binding portion” of an antibody, and the like, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated CDR, such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In some embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (X) V_{L}—C_{H}3; (Xi) V_{L}-C_{H}1-C_{H}2; (Xii) V_{L}-C_{H}1-C_{H}2-C_{H}3; (Xiii) V_{L}-C_{H}2-C_{H}3; and (xiv) V_{L}-C_{L}. In any configuration of variable and constant domains, including any of the exemplary configurations set forth above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present disclosure may comprise a homo-dimer or hetero-dimer (or other multimer) of any of the variable and constant domain configurations set forth above in non-covalent association with one another and/or with one or more monomeric V_{H} or V_{L} domain (e.g., by disulfide bond(s)).

In some embodiments, the antigen-binding domain comprises three heavy chain CDRs (HCDR1, HCDR2, and HCDR3) and three light chain CDRs (LCDR1, LCDR2, and LCDR3), wherein: HCDR1 comprises an amino acid sequence of SEQ ID NO: 2, 12, or 21; HCDR2 comprises an amino acid sequence of SEQ ID NO: 3, 13, or 22; HCDR3 comprises an amino acid sequence of SEQ ID NO: 4, 14, or 23; LCDR1 comprises an amino acid sequence of SEQ ID NO: 6 or 16; LCDR2 comprises an amino acid sequence of SEQ ID NO: 7; and LCDR3 comprises an amino acid sequence of SEQ ID NO: 8 or 17.

In some embodiments, the antigen-binding domain comprises HCDR1, HCDR2, HCDR3, LCDR1, LCDR2, and LCDR3 comprising respective amino acid sequences of (i) SEQ ID NOs: 2, 3, 4, 6, 7, and 8; (ii) SEQ ID NOs: 12, 13, 14, 16, 7, and 17; or (iii) SEQ ID NOs: 21, 22, 23, 6, 7, and 8.

In some embodiments, the antigen-binding domain comprises HCDR1, HCDR2, HCDR3, LCDR1, LCDR2, and LCDR3 comprising the amino acid sequences of SEQ ID NOs: 21, 22, 23, 6, 7, and 8, respectively.

In some embodiments, the antigen-binding domain comprises a HCVR comprising an amino acid sequence of SEQ ID NO: 1, 11, and 20 or an amino acid sequence having 80%, 85%, 90%, 95%, 97%, 98% or 99% sequence identity to SEQ ID NO: 1, 11, and 20; and a LCVR comprising an amino acid sequence of SEQ ID NO: 5 or 15 or an amino acid sequence having 80%, 85%, 90%, 95%, 97%, 98% or 99% sequence identity to SEQ ID NO: 5 or 15. Sequence identity can be calculated using an algorithm, for example, the Needleman Wunsch algorithm (Needleman et al., J. Mol. Biol. 48:443-453 (1970)) for global alignment, or the Smith Waterman algorithm (Smith et al., J. Mol. Biol., 147:195-197 (1981)) for local alignment. Another suitable algorithm is described by Dufresne et al., Nature Biotechnology, 20:1269-1271 (2002)) and is used in the software GenePAST (GQ Life Sciences, Inc.; Boston, MA).

In some embodiments, the antigen-binding domain comprises a HCVR/LCVR amino acid sequence pair selected from SEQ ID NOs: 1/5, 11/15, and 20/5.

In some embodiments, the fusion protein further comprises a heavy chain constant region of SEQ ID NO: 26.

In some embodiments, the fusion protein comprises a heavy chain and a light chain, wherein the heavy chain comprises the amino acid sequence of SEQ ID NO: 9, 18, or 24 or an amino acid sequence having 80%, 85%, 90%, 95%, 97%, 98% or 99% sequence identity to SEQ ID NO: 9, 18, or 24; and the light chain comprises the amino acid sequence of SEQ ID NO: 10, 19, or 25 or an amino acid sequence having 80%, 85%, 90%, 95%, 97%, 98% or 99% sequence identity to SEQ ID NO: 10, 19, or 25.

In some embodiments, the fusion protein comprises a heavy chain/light chain sequence pair comprising the amino acid sequences of SEQ ID NOs: 9/10, 18/19, or 24/25. In some embodiments, the fusion protein comprises a heavy chain/light chain sequence pair comprising the amino acid sequences of SEQ ID NOs: 24 and 25.

In some embodiments, the IL2 moiety comprises (i) IL2 or a fragment thereof; and (ii) IL2 receptor alpha (“IL2Rα” or “IL2Ra”) or a fragment thereof.

In some embodiments, the IL2 moiety may include a wild type (e.g., human wild type) or variant IL2 domain that is fused to an IL2 binding domain of IL2Ra, optionally via a linker. In some embodiments, the IL2 binding domain of IL2Ra of a fragment thereof is bound at its C-terminus via a linker to the IL2 (wild type or variant) domain or fragment thereof.

As used herein, a “wild type” form of IL2 is a form of IL2 that is otherwise the same as a mutant IL2 polypeptide except that the wild type form has a wild type amino acid at each amino acid position of the mutant IL2 polypeptide. For example, if the IL2 mutant is the full-length IL2 (i.e., IL2 not fused or conjugated to any other molecule), the wild type form of this mutant is full-length native IL2.

In some embodiments, the IL2 or fragment thereof comprises the amino acid sequence of SEQ ID NO: 29. In some embodiments, the IL2 moiety comprises the amino acid sequence of SEQ ID NO: 27.

The targeted immunocytokine may include one or more linkers (e.g., peptide linker or non-peptide linker) connecting the various components of the molecule. In some embodiments, two or more components of the targeted immunocytokine are connected to one another by a peptide linker. By way of a non-limiting example, linkers can be used to connect (a) an IL2 moiety and an antigen-binding domain of a checkpoint inhibitor; (b) different domains within an IL2 moiety (e.g., an IL2 domain and an IL2Ra domain); or (c) different domains within an antigen-binding moiety (e.g., different components of anti-PD-1 antigen-binding domain).

Examples of flexible linkers that may be used in the disclosed targeted immunocytokine include those disclosed in Chen et al., Adv Drug Deliv Rev., 65(10):1357-69 (2013) and Klein et al., Protein Engineering, Design & Selection, 27(10):325-30 (2014). Particularly useful flexible linkers are or comprise repeats of glycines and serines, e.g., a monomer or multimer of GnS or SGn, where n is an integer from 1 to 10, e.g., 1, 2, 3, 4, 5, 6, 7, 8, 9, or 10. In some embodiments, the linker is or comprises a monomer or multimer of repeating G4S (GGGGS; SEQ ID NO: 32), e.g., (GGGGS)n.

In some embodiments, the IL2 moiety and the antigen-binding moiety are connected via a linker that comprises an amino acid sequence of one or more repeats of GGGGS (SEQ ID NO: 32). In some embodiments, the linker comprises an amino acid sequence of SEQ ID NO: 30 or 31. In some embodiments, the IL2 moiety is linked to the C-terminus of the antigen-binding moiety via a peptide linker. In some embodiments, the linker comprises an amino acid sequence of SEQ ID NO: 30.

In some embodiments, the targeted immunocytokine comprises a dimeric fusion protein. In some embodiments, the dimeric fusion protein is a homodimeric fusion protein, wherein each constituent monomer comprises a fusion protein described herein. In some embodiments, the monomers of the dimeric fusion protein dimerize to each other through the heavy chain constant region of each monomer. In one preferred embodiment, the IL2 of a first monomeric component binds to IL2Ra comprised in the second monomeric component of a dimeric protein.

The targeted immunocytokine of the present disclosure exhibits attenuated binding to IL2Rα, IL2Rβ and IL2Rγ. In some embodiments, the targeted immunocytokine does not compete with REGN2810, pembrolizumab or nivolumab. In some embodiments, the targeted immunocytokine exhibits reduced activity in activating human IL2Rα/β/γ trimeric and IL2Rβ/γ dimeric receptor complexes as compared to IL2 and increased activity in activating human IL2Rα/β/γ trimeric and IL2Rβ/γ dimeric receptor complexes as compared to a non-targeted IL2Rα-IL2 construct. In some embodiments, the targeted immunocytokine exhibits increased activity in stimulating antigen-activated T cells as measured by a level of IFN-γ release as compared to a wild type human IL2.

In some embodiments, the targeted immunocytokine is an anti-PD1-IL2Ra-IL2 fusion protein.

Combination Therapies

In general, the methods of the present disclosure include administering to a subject with cancer a combination therapy comprising a therapeutically effective amount of an ACT and a therapeutically effective amount of a targeted immunocytokine. In some embodiments, the disclosed combination therapy increases the efficacy of ACT administered to a subject with cancer as compared to a subject treated with the ACT as monotherapy or treated with the ACT in combination with a non-targeted immunocytokine, thereby more effectively treating the cancer.

With respect to pharmaceutical compositions, the disclosed ACT and/or targeted immunocytokine may be formulated with one or more carriers, excipients and/or diluents. In some embodiments, the targeted immunocytokine may be formulated in the form of a fusion protein (e.g., dimeric fusion protein) with one or more carriers, excipients and/or diluents. Pharmaceutical compositions comprising the disclosed ACT and/or targeted immunocytokine may be formulated for specific uses, such as for veterinary uses or pharmaceutical uses in humans. The form of the composition (e.g., dry powder, liquid formulation, etc.) and the excipients, diluents and/or carriers used will depend upon the intended therapeutic use and desired mode of administration of the ACT and/or targeted immunocytokine.

A pharmaceutical composition of the present disclosure may contain either or both of the ACT and targeted immunocytokine. Such pharmaceutical compositions may be administered to a subject by a variety of routes such as orally, transdermally, subcutaneously, intranasally, intravenously, intramuscularly, intratumorally, intrathecally, topically, or locally. In some embodiments, the pharmaceutical composition is administered to the subject intravenously or subcutaneously. Pharmaceutical compositions can be conveniently presented in unit dosage forms containing a predetermined amount of the disclosed ACT and/or targeted immunocytokine per dose.

In some embodiments, the disclosed methods further include administration of an additional therapeutic agent or therapy. Non-limiting examples of the additional therapeutic agent or therapy include radiation, surgery, a cancer vaccine, a PD-L1 inhibitor (e.g., an anti-PD-L1 antibody), a LAG-3 inhibitor, a CTLA-4 inhibitor (e.g., ipilimumab), a TIM3 inhibitor, a BTLA inhibitor, a TIGIT inhibitor, a CD47 inhibitor, an antagonist of another T cell co-inhibitor or ligand (e.g., an antibody to LAIR1, CD160,g or VISTA), an indoleamine-2,3-dioxygenase (IDO) inhibitor, a vascular endothelial growth factor (VEGF) antagonist [e.g., a “VEGF-Trap” such as aflibercept or other VEGF-inhibiting fusion protein as set forth in U.S. Pat. No. 7,087,411, or an anti-VEGF antibody or antigen binding fragment thereof (e.g., bevacizumab, or ranibizumab) or a small molecule kinase inhibitor of VEGF receptor (e.g., sunitinib, sorafenib, or pazopanib)], an Ang2 inhibitor (e.g., nesvacumab), a transforming growth factor beta (TGFβ) inhibitor, an epidermal growth factor receptor (EGFR) inhibitor (e.g., erlotinib, cetuximab), an agonist to a co-stimulatory receptor (e.g., an agonist to glucocorticoid-induced TNFR-related protein), an antibody to a tumor-specific antigen (e.g., CA9, CA125, melanoma-associated antigen 3 (MAGE3), carcinoembryonic antigen (CEA), vimentin, tumor-M2-PK, prostate-specific antigen (PSA), mucin-1, MART-1, and CA19-9), a vaccine (e.g., Bacillus Calmette-Guerin, a cancer vaccine), an adjuvant to increase antigen presentation (e.g., granulocyte-macrophage colony-stimulating factor), a cytotoxin, a chemotherapeutic agent (e.g., dacarbazine, temozolomide, cyclophosphamide, docetaxel, doxorubicin, daunorubicin, cisplatin, carboplatin, gemcitabine, methotrexate, mitoxantrone, oxaliplatin, paclitaxel, and vincristine), radiotherapy, an IL-6R inhibitor (e.g., sarilumab), an IL-4R inhibitor (e.g., dupilumab), an IL-10 inhibitor, a cytokine such as IL-2, IL-7, IL-21, and IL-15, an antibody-drug conjugate (ADC) (e.g., anti-CD19-DM4 ADC, and anti-DS6-DM4 ADC), an anti-inflammatory drug (e.g., corticosteroids, and non-steroidal anti-inflammatory drugs), a dietary supplement such as anti-oxidants, and combinations thereof.

In some embodiments, the additional therapeutic agent or therapy comprises an anti-cancer drug. As used herein, an “anti-cancer drug” means any agent useful to treat cancer including, but not limited to, cytotoxins and agents such as antimetabolites, alkylating agents, anthracyclines, antibiotics, antimitotic agents, procarbazine, hydroxyurea, asparaginase, corticosteroids, mytotane (O,P′-(DDD)), biologics (e.g., antibodies and interferons) and radioactive agents. As used herein, “a cytotoxin or cytotoxic agent” also refers to a chemotherapeutic agent and means any agent that is detrimental to cells. Examples include Taxol® (paclitaxel), temozolamide, cytochalasin B, gramicidin D, ethidium bromide, emetine, cisplatin, mitomycin, etoposide, tenoposide, vincristine, vinbiastine, coichicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof.

As used herein, a “therapeutic agent” refers to a molecule or compound that confers some beneficial effect upon administration to a subject. The beneficial effect may include enablement of diagnostic determinations; amelioration of a disease, symptom, disorder or pathological condition; reducing or preventing the onset of a disease, symptom, disorder or condition; and generally counteracting a disease, symptom, disorder or pathological condition.

In some embodiments, the combined administration of the ACT and targeted immunocytokine with an additional therapeutic agent or therapy leads to improved anti-tumor efficacy, reduced side effects of one or both of the primary therapies, and/or reduced dosage of one or both of the primary therapies.

The present disclosure also provides kits comprising the disclosed ACT (e.g., immune cells modified with an anti-TAA TCR or CAR) and targeted immunocytokine (e.g., a fusion protein comprising an immunoglobulin antigen-binding domain of a checkpoint inhibitor and an IL-2 moiety). Kits typically include a label indicating the intended use of the contents of the kit and instructions for use. As used herein, the term “label” includes any writing, or recorded material supplied on, in or with the kit, or that otherwise accompanies the kit. In some embodiments, the present disclosure provides a kit for treating a subject afflicted with a cancer, wherein the kit includes: a therapeutically effective dosage of a disclosed ACT; a therapeutically effective dosage of a disclosed targeted immunocytokine; and (b) instructions for using the combination of dosages in any of the methods disclosed herein.

Administration Regimens

The present disclosure includes methods that comprise administering to a subject with cancer a combination of the disclosed ACT and/or the disclosed targeted immunocytokine at a dosing frequency that achieves a therapeutic response. In some embodiments, the disclosed ACT is administered to the subject in one or more doses administered about four times a week, twice a week, once a week, once every two weeks, once every three weeks, once every four weeks, once every five weeks, once every six weeks, once every eight weeks, once every twelve weeks, or less frequently so long as a therapeutic response is achieved.

In some embodiments, the disclosed targeted immunocytokine is administered to the subject in one or more doses administered about four times a week, twice a week, once a week, once every two weeks, once every three weeks, once every four weeks, once every five weeks, once every six weeks, once every eight weeks, once every twelve weeks, or less frequently so long as a therapeutic response is achieved.

In the disclosed methods, a disclosed ACT is administered to the subject in combination with a disclosed targeted immunocytokine. As used herein, the expression “in combination with” means that the ACT is administered before, after, or concurrently with the targeted immunocytokine. This expression includes sequential or concurrent administration of the ACT and targeted immunocytokine.

In some embodiments, when the ACT is administered “before” the targeted immunocytokine, the ACT may be administered more than 12 weeks, about 12 weeks, about 11 weeks, about 10 weeks, about 9 weeks, about 8 weeks, about 7 weeks, about 6 weeks, about 5 weeks, about 4 weeks, about 3 weeks, about 2 weeks, about 1 week, about 150 hours, about 100 hours, about 72 hours, about 60 hours, about 48 hours, about 36 hours, about 24 hours, about 12 hours, about 10 hours, about 8 hours, about 6 hours, about 4 hours, about 2 hours, about 1 hour, about 30 minutes, about 15 minutes or about 10 minutes prior to the administration of the targeted immunocytokine.

In some embodiments, when the ACT is administered “after” the targeted immunocytokine, the ACT may be administered about 10 minutes, about 15 minutes, about 30 minutes, about 1 hour, about 2 hours, about 4 hours, about 6 hours, about 8 hours, about 10 hours, about 12 hours, about 24 hours, about 36 hours, about 48 hours, about 60 hours, about 72 hours, about 1 week, about 2 weeks, about 3 weeks, about 4 weeks, about 5 weeks, about 5 weeks, about 7 weeks, about 8 weeks, about 9 weeks, about 10 weeks, about 11 weeks, about 12 weeks, or more than 12 weeks after the administration of the targeted immunocytokine.

As used herein, “concurrent” administration means that the ACT and targeted immunocytokine are administered to the subject in a single dosage form (e.g., co-formulated) or in separate dosage forms administered to the subject within about 30 minutes or less of each other (i.e., before, after, or at the same time), such as about 15 minutes or less, or about 5 minutes or less. If administered in separate dosage forms, each dosage form may be administered via the same route (e.g., both administered intravenously, subcutaneously, etc.); or, alternatively, each dosage form may be administered via a different route. In any event, administering the components in a single dosage from, in separate dosage forms by the same route, or in separate dosage forms by different routes are all considered “concurrent” administration” for purposes of the present disclosure.

As used herein, “sequential” administration means that each dose of a selected therapy is administered to the subject at a different point in time, e.g., on different days separated by a predetermined interval (e.g., hours, days, weeks, or months). For illustrative purposes, sequential administration may include administering an initial dose of the ACT (or targeted immunocytokine), followed by one or more secondary doses the targeted immunocytokine (or ACT), optionally followed by one or more tertiary doses of the ACT (or targeted immunocytokine). For illustrative purposes, sequential administration may include administering to the subject an initial dose of the ACT (or targeted immunocytokine), followed by one or more secondary doses of the targeted immunocytokine (or ACT), and optionally followed by one or more tertiary doses of the targeted immunocytokine (or ACT).

As used herein, “initial” dose, “secondary” dose, and “tertiary” dose refer to the temporal sequence of administration. Thus, the “initial” dose is the dose which is administered at the beginning of the treatment regimen (also referred to as the “baseline dose”); “secondary” doses are administered after the initial dose; and “tertiary” doses are administered after the secondary doses. The initial, secondary, and tertiary doses may all contain the same amount of the selected therapy or may contain different amounts of the selected therapy.

Dosage

In general, the amount of ACT and/or targeted immunocytokine administered to a subject according to the methods of the present disclosure is a therapeutically effective amount. As used herein, “therapeutically effective amount” means an amount of the targeted immunocytokine in combination with the ACT that results in one or more of: (a) a reduction in the severity or duration of a symptom of a cancer; (b) enhanced inhibition of tumor growth, or an increase in tumor necrosis, tumor shrinkage and/or tumor disappearance; (c) delay in tumor growth and development; (d) inhibit or retard or stop tumor metastasis; (e) prevention of recurrence of tumor growth; (f) increase in survival of a subject with a cancer; and/or (g) a reduction in the use or need for conventional anti-cancer therapy (e.g., reduced or eliminated use of chemotherapeutic or cytotoxic agents) as compared to an untreated subject or a subject treated with ACT as monotherapy.

In some embodiments, a therapeutically effective amount of the ACT may comprise immune effector cells expressing a modified TCR or CAR against a tumor-associated antigen administered in an amount of about 1×10⁶ or more, 2×10⁶ or more, 3×10⁶ or more, 4×10⁶ or more, 5×10⁶ or more, 6×10⁶ or more, 7×10⁶ or more, 8×10⁶ or more, 9×10⁶ or more, 1×10⁷ or more, 2×10⁷ or more, 3×10⁷ or more, 4×10⁷ or more, 5×10⁷ or more, 6×10⁷ or more, 7×10⁷ or more, 8×10⁷ or more, 9×10⁷ or more, 1×10⁸ or more, 2×10⁸ or more, 3×10⁸ or more, 4×10⁸ or more, 5×10⁸ or more, 6×10⁸ or more, 7×10⁸ or more, 8×10⁸ or more, 9×10⁸ or more, 1×10⁹ or more, 2×10⁹ or more, 3×10⁹ or more, 4×10⁹ or more, 5×10⁹ or more, 6×10⁹ or more, 7×10⁹ or more, 8×10⁹ or more, 9×10⁹ or more cells. In some embodiments, the amount of the ACT administered to the subject comprises 1×10⁶ or more immune cells expressing a modified TCR or CAR against a tumor-associated antigen.

In some embodiments, a therapeutically effective amount of the targeted immunocytokine may be from about 0.05 mg to about 600 mg, e.g., about 0.05 mg, about 0.1 mg, about 1.0 mg, about 1.5 mg, about 2.0 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, or about 600 mg, of the targeted immunocytokine.

In some embodiments, the amount of the targeted immunocytokine administered to the subject comprises 0.005 mg/kg to 10 mg/kg of the subject's body weight, such as 0.01 mg/kg to 10 mg/kg, 0.02 mg/kg to 10 mg/kg, 0.03 mg/kg to 10 mg/kg, 0.04 mg/kg to 10 mg/kg, 0.05 mg/kg to 10 mg/kg, 0.06 mg/kg to 10 mg/kg, 0.07 mg/kg to 10 mg/kg, 0.08 mg/kg to 10 mg/kg, 0.09 mg/kg to 10 mg/kg, 0.1 mg/kg to 10 mg/kg, 0.2 mg/kg to 10 mg/kg, 0.3 mg/kg to 10 mg/kg, 0.4 mg/kg to 10 mg/kg, 0.5 mg/kg to 10 mg/kg, 0.6 mg/kg to 10 mg/kg, 0.7 mg/kg to 10 mg/kg, 0.8 mg/kg to 10 mg/kg, 0.9 mg/kg to 10 mg/kg, 1 mg/kg to 10 mg/kg, 0.005 mg/kg to 5 mg/kg of the subject's body weight, such as 0.01 mg/kg to 5 mg/kg, 0.02 mg/kg to 5 mg/kg, 0.03 mg/kg to 5 mg/kg, 0.04 mg/kg to 5 mg/kg, 0.05 mg/kg to 10 mg/kg, 0.06 mg/kg to 5 mg/kg, 0.07 mg/kg to 5 mg/kg, 0.08 mg/kg to 5 mg/kg, 0.09 mg/kg to 5 mg/kg, 0.1 mg/kg to 10 mg/kg, 0.2 mg/kg to 5 mg/kg, 0.3 mg/kg to 5 mg/kg, 0.4 mg/kg to 5 mg/kg, 0.5 mg/kg to 5 mg/kg, 0.6 mg/kg to 5 mg/kg, 0.7 mg/kg to 5 mg/kg, 0.8 mg/kg to 5 mg/kg, 0.9 mg/kg to 5 mg/kg, or 1 mg/kg to 5 mg/kg.

As used herein, the singular forms “a,” “an,” and “the” include plural reference unless the context clearly dictates otherwise. As used herein, the terms “including,” “comprising,” “containing,” or “having” and variations thereof are meant to encompass the items listed thereafter and equivalents thereof as well as additional subject matter unless otherwise noted. As used herein, the phrases “in one embodiment,” “in various embodiments,” “in some embodiments,” and the like are used repeatedly. Such phrases do not necessarily refer to the same embodiment, but they may unless the context dictates otherwise. As used herein, the terms “and/or” or “/” means any one of the items, any combination of the items, or all of the items with which this term is associated.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

The present disclosure merely illustrates the principles of the disclosed technology. Any examples set forth in this specification are not intended to be limiting and merely set forth some of the many possible embodiments for the appended claims. Those skilled in the art will readily recognize various modifications and changes that may be made without following the example embodiments and applications illustrated and described herein, and without departing from the true spirit and scope of the following claims. All references cited and/or discussed in this specification are incorporated herein by reference in their entireties and to the same extent as if each reference was individually incorporated by reference.

### EXAMPLES

The disclosed technology is next described by means of the following examples. The use of these and other examples anywhere in the specification is illustrative only, and in no way limits the scope and meaning of the invention or of any exemplified form. Likewise, the invention is not limited to any particular preferred embodiments described herein. Indeed, modifications and variations of the invention may be apparent to those skilled in the art upon reading this specification, and can be made without departing from its spirit and scope. The invention is therefore to be limited only by the terms of the claims, along with the full scope of equivalents to which the claims are entitled. Also, while efforts have been made to ensure accuracy with respect to numbers used (e.g., amounts, temperature, etc.), some experimental errors and deviations should be accounted for. Unless indicated otherwise, parts are parts by weight, molecular weight is average molecular weight, temperature is in degrees Centigrade, room temperature is about 25° C., and pressure is at or near atmospheric.

### Example 1: Generation of Anti-PD1-IL2Ra-IL2 Fusion Proteins

Three anti-PD1-IL2Ra-IL2 fusion proteins were generated by expressing a first polynucleotide sequence encoding a heavy chain of an anti-PD-1 antibody linked to the N-terminus of a IL2 moiety and a second polynucleotide sequence encoding a light chain of the anti-PD-1 antibody in host cells. The IL2 moiety includes IL2 linked to the C-terminus of IL2Ra. The first polynucleotide sequence and the second polynucleotide sequence can be carried on the same or different expression vectors. See U.S. patent application Ser. No. 17/806,566.

Table 1 sets forth the amino acid sequence identifiers of the three anti-PD1-IL2Ra-IL2 fusion proteins.

TABLE 1
Amino acid identifiers of anti-PD1-IL2Ra-IL2 fusion proteins

| | SEQ ID NOs | | | | | | | | | | |
|---|---|---|---|---|---|---|---|---|---|---|---|
| | | | | | | | | | | | IL2 |
| ID | HCVR | HCDR1 | HCDR2 | HCDR3 | LCVR | LCDR1 | LCDR2 | LCDR3 | HC | LC | moiety |
| REGN10595 | 1 | 2 | 3 | 4 | 5 | 6 | 7 | 8 | 9 | 10 | 27 |
| REGN10486 | 11 | 12 | 13 | 14 | 15 | 16 | 7 | 17 | 18 | 19 | 27 |
| REGN10597 | 20 | 21 | 22 | 23 | 5 | 6 | 7 | 8 | 24 | 25 | 27 |

The IL2 moiety (SEQ ID NO: 27) includes an IL2 (SEQ ID NO: 29) linked to the C-terminus of an IL2Ra (SEQ ID NO: 28). The IL2 moiety (SEQ ID NO: 27) is connected to the C-terminus of the heavy chain constant region (SEQ ID NO: 26) of the anti-PD-1 antibody via a linker comprising an amino acid sequence of SEQ ID NO: 30.

For REGN10595, the heavy chain (HC) (SEQ ID NO: 9) includes the amino acid sequences of the HCVR (SEQ ID NO: 1), and the heavy chain constant region (SEQ ID NO: 26) linked to the IL2 moiety (SEQ ID NO: 27) via a linker (SEQ ID NO: 30).

For REGN10486, the heavy chain (HC) (SEQ ID NO: 18) includes the amino acid sequences of the HCVR (SEQ ID NO: 11)) and the heavy chain constant region (SEQ ID NO: 26) linked to the IL2 moiety (SEQ ID NO: 27) via a linker (SEQ ID NO: 30).

For REGN10597, the heavy chain (HC) (SEQ ID NO: 24) includes the amino acid sequences of the HCVR (SEQ ID NO: 20) and the heavy chain constant region (SEQ ID NO: 26) linked to the IL2 moiety (SEQ ID NO: 27) via a linker (SEQ ID NO: 30).

Table 2 sets forth the amino acid sequences of the three anti-PD1-IL2Ra-IL2 fusion proteins.

TABLE 2
Amino acid sequences of anti-PD1-IL2Ra-IL2

| SEQ | | |
|---|---|---|
| ID | | |
| NO | SEQUENCE | INFORMATION |
| 1 | QVQLVQSGTEVRKPGSSVKVSCKTS | VH; |
| | GVTFNNYAITWVRQAPGQGLEWMGG | REGN10595 |
| | IIPVFSPPNYAQKFQGRVTITADES | |
| | TNTAYMELNSLRSDDTAIYFCAREG | |
| | ERGYTYGYDYWGQGTLVTVSS | |
| 2 | GVTFNNYA | HCDR1; |
| | | REGN10595 |
| 3 | IIPVFSPP | HCDR2; |
| | | REGN10595 |
| 4 | AREGERGYTYGYDY | HCDR3; |
| | | REGN10595 |
| 5 | DIQMTQSPSSLSASVGDRVTITCRA | VL |
| | SQSISSYLNWYQQKPGKAPKLLIYA | REGN10595 |
| | ASSLQSGVPSRFSGSGSGTDFTLTI | |
| | SSLQPEDFATYYCQQSYSTPPITFG | |
| | QGTRLEIK | |
| 6 | QSISSY | LCDR1; |
| | | REGN10595 |
| 7 | AAS | LCDR2; |
| | | REGN10595 |
| 8 | QQSYSTPPIT | LCDR3; |
| | | REGN10595 |
| 9 | QVQLVQSGTEVRKPGSSVKVSCKTS | HC; |
| | GVTFNNYAITWVRQAPGQGLEWMGG | REGN10595 |
| | IIPVFSPPNYAQKFQGRVTITADES | |
| | TNTAYMELNSLRSDDTAIYFCAREG | |
| | ERGYTYGYDYWGQGTLVTVSSASTK | |
| | GPSVFPLAPCSRSTSESTAALGCLV | |
| | KDYFPEPVTVSWNSGALTSGVHTFP | |
| | AVLQSSGLYSLSSVVTVPSSSLGTK | |
| | TYTCNVDHKPSNTKVDKRVESKYGP | |
| | PCPPCPAPPVAGPSVFLFPPKPKDT | |
| | LMISRTPEVTCVVVDVSQEDPEVQF | |
| | NWYVDGVEVHNAKTKPREEQFNSTY | |
| | RVVSVLTVLHQDWLNGKEYKCKVSN | |
| | KGLPSSIEKTISKAKGQPREPQVYT | |
| | LPPSQEEMTKNQVSLTCLVKGFYPS | |
| | DIAVEWESNGQPENNYKTTPPVLDS | |
| | DGSFFLYSRLTVDKSRWQEGNVFSC | |
| | SVMHEALHNHYTQKSLSLSLGK | |
| 10 | DIQMTQSPSSLSASVGDRVTITCRA | LC; |
| | SQSISSYLNWYQQKPGKAPKLLIYA | REGN10595 |
| | ASSLQSGVPSRFSGSGSGTDFTLTI | |
| | SSLQPEDFATYYCQQSYSTPPITFG | |
| | QGTRLEIKRTVAAPSVFIFPPSDEQ | |
| | LKSGTASVVCLLNNFYPREAKVQWK | |
| | VDNALQSGNSQESVTEQDSKDSTYS | |
| | LSSTLTLSKADYEKHKVYACEVTHQ | |
| | GLSSPVTKSFNRGEC | |
| 11 | EVQLLESGGVLVQPGGSLRLSCAAS | VH; |
| | GFTFSNFGMTWVRQAPGKGLEWVSG | REGN10486 |
| | ISGGGRDTYFADSVKGRFTISRDNS | |
| | KNTLYLQMNSLKGEDTAVYYCVKWG | |
| | NIYFDYWGQGTLVTVSS | |
| 12 | GFTFSNFG | HCDR1; |
| | | REGN10486 |
| 13 | ISGGGRDT | HCDR2; |
| | | REGN10486 |
| 14 | VKWGNIYFDY | HCDR3; |
| | | REGN10486 |
| 15 | DIQMTQSPSSLSASVGDSITITCRA | VL; |
| | SLSINTFLNWYQQKPGKAPNLLIYA | REGN10486 |
| | ASSLHGGVPSRFSGSGSGTDFTLTI | |
| | RTLQPEDFATYYCQQSSNTPFTFGP | |
| | GTVVDFR | |
| 16 | LSINTF | LCDR1; |
| | | REGN10486 |
| 7 | AAS | LCDR2; |
| | | REGN10486 |
| 17 | QQSSNTPFT | LCDR3; |
| | | REGN10486 |
| 18 | EVQLLESGGVLVQPGGSLRLSCAAS | HC; |
| | GFTFSNFGMTWVRQAPGKGLEWVSG | REGN10486 |
| | ISGGGRDTYFADSVKGRFTISRDNS | |
| | KNTLYLQMNSLKGEDTAVYYCVKWG | |
| | NIYFDYWGQGTLVTVSSASTKGPSV | |
| | FPLAPCSRSTSESTAALGCLVKDYF | |
| | PEPVTVSWNSGALTSGVHTFPAVLQ | |
| | SSGLYSLSSVVTVPSSSLGTKTYTC | |
| | NVDHKPSNTKVDKRVESKYGPPCPP | |
| | CPAPPVAGPSVFLFPPKPKDTLMIS | |
| | RTPEVTCVVVDVSQEDPEVQFNWYV | |
| | DGVEVHNAKTKPREEQFNSTYRVVS | |
| | VLTVLHQDWLNGKEYKCKVSNKGLP | |
| | SSIEKTISKAKGQPREPQVYTLPPS | |
| | QEEMTKNQVSLTCLVKGFYPSDIAV | |
| | EWESNGQPENNYKTTPPVLDSDGSF | |
| | FLYSRLTVDKSRWQEGNVFSCSVMH | |
| | EALHNHYTQKSLSLSLGK | |
| 19 | DIQMTQSPSSLSASVGDSITITCRA | LC; |
| | SLSINTFLNWYQQKPGKAPNLLIYA | REGN10486 |
| | ASSLHGGVPSRFSGSGSGTDFTLTI | |
| | RTLQPEDFATYYCQQSSNTPFTFGP | |
| | GTVVDFRRTVAAPSVFIFPPSDEQL | |
| | KSGTASVVCLLNNFYPREAKVQWKV | |
| | DNALQSGNSQESVTEQDSKDSTYSL | |
| | SSTLTLSKADYEKHKVYACEVTHQG | |
| | LSSPVTKSFNRGEC | |
| 20 | QVQLVQSGAEVKRPGSSVKVSCKVS | VH; |
| | GVTFRNFAIIWVRQAPGQGLEWMGG | REGN10597 |
| | IIPFFSAANYAQSFQGRVTITPDES | |
| | TSTAFMELASLRSEDTAVYYCAREG | |
| | ERGHTYGFDYWGQGTLVTVSS | |
| 21 | GVTFRNFA | HCDR1; |
| | | REGN10597 |
| 22 | IIPFFSAA | HCDR2; |
| | | REGN10597 |
| 23 | AREGERGHTYGFDY | HCDR3; |
| | | REGN10597 |
| 5 | DIQMTQSPSSLSASVGDRVTITCRA | VL; |
| | SQSISSYLNWYQQKPGKAPKLLIYA | REGN10597 |
| | ASSLQSGVPSRFSGSGSGTDFTLTI | |
| | SSLQPEDFATYYCQQSYSTPPITFG | |
| | QGTRLEIK | |
| 6 | QSISSY | LCDR1; |
| | | REGN10597 |
| 7 | AAS | LCDR2; |
| | | REGN10597 |
| 8 | QQSYSTPPIT | LCDR3; |
| | | REGN10597 |
| 24 | QVQLVQSGAEVKRPGSSVKVSCKVS | HC; |
| | GVTFRNFAIIWVRQAPGQGLEWMGG | REGN10597 |
| | IIPFFSAANYAQSFQGRVTITPDES | |
| | TSTAFMELASLRSEDTAVYYCAREG | |
| | ERGHTYGFDYWGQGTLVTVSSASTK | |
| | GPSVFPLAPCSRSTSESTAALGCLV | |
| | KDYFPEPVTVSWNSGALTSGVHTFP | |
| | AVLQSSGLYSLSSVVTVPSSSLGTK | |
| | TYTCNVDHKPSNTKVDKRVESKYGP | |
| | PCPPCPAPPVAGPSVFLFPPKPKDT | |
| | LMISRTPEVTCVVVDVSQEDPEVQF | |
| | NWYVDGVEVHNAKTKPREEQFNSTY | |
| | RVVSVLTVLHQDWLNGKEYKCKVSN | |
| | KGLPSSIEKTISKAKGQPREPQVYT | |
| | LPPSQEEMTKNQVSLTCLVKGFYPS | |
| | DIAVEWESNGQPENNYKTTPPVLDS | |
| | DGSFFLYSRLTVDKSRWQEGNVFSC | |
| | SVMHEALHNHYTQKSLSLSLGK | |
| 25 | DIQMTQSPSSLSASVGDRVTITCRA | LC; |
| | SQSISSYLNWYQQKPGKAPKLLIYA | REGN10597 |
| | ASSLQSGVPSRFSGSGSGTDFTLTI | |
| | SSLQPEDFATYYCQQSYSTPPITFG | |
| | QGTRLEIKRTVAAPSVFIFPPSDEQ | |
| | LKSGTASVVCLLNNFYPREAKVQWK | |
| | VDNALQSGNSQESVTEQDSKDSTYS | |
| | LSSTLTLSKADYEKHKVYACEVTHQ | |
| | GLSSPVTKSFNRGEC | |
| 26 | ASTKGPSVFPLAPCSRSTSESTAAL | Heavy chain |
| | GCLVKDYFPEPVTVSWNSGALTSGV | constant |
| | HTFPAVLQSSGLYSLSSVVTVPSSS | region |
| | LGTKTYTCNVDHKPSNTKVDKRVES | |
| | KYGPPCPPCPAPPVAGPSVFLFPPK | |
| | PKDTLMISRTPEVTCVVVDVSQEDP | |
| | EVQFNWYVDGVEVHNAKTKPREEQF | |
| | NSTYRVVSVLTVLHQDWLNGKEYKC | |
| | KVSNKGLPSSIEKTISKAKGQPREP | |
| | QVYTLPPSQEEMTKNQVSLTCLVKG | |
| | FYPSDIAVEWESNGQPENNYKTTPP | |
| | VLDSDGSFFLYSRLTVDKSRWQEGN | |
| | VFSCSVMHEALHNHYTQKSLSLSLG | |
| | K | |
| 27 | ELCDDDPPEIPHATFKAMAYKEGTM | IL2 moiety |
| | LNCECKRGFRRIKSGSLYMLCTGNS | (IL2Ra+IL2) |
| | SHSSWDNQCQCTSSATRNTTKQVTP | |
| | QPEEQKERKTTEMQSPMQPVDQASL | |
| | PGHCREPPPWENEATERIYHFVVGQ | |
| | MVYYQCVQGYRALHRGPAESVCKMT | |
| | HGKTRWTQPQLICTGGGGGSGGGGS | |
| | GGGGSGGGGSGGGGSAPTSSSTKKT | |
| | QLQLEHLLLDLQMILNGINNYKNPK | |
| | LTRMLTFKFYMPKKATELKHLQCLE | |
| | EELKPLEEVLNLAQSKNFHLRPRDL | |
| | ISNINVIVLELKGSETTFMCEYADE | |
| | TATIVEFLNRWITFCQSIISTLT | |
| 28 | ELCDDDPPEIPHATFKAMAYKEGTM | hIL2Ra |
| | LNCECKRGFRRIKSGSLYMLCTGNS | |
| | SHSSWDNQCQCTSSATRNTTKQVTP | |
| | QPEEQKERKTTEMQSPMQPVDQASL | |
| | PGHCREPPPWENEATERIYHFVVGQ | |
| | MVYYQCVQGYRALHRGPAESVCKMT | |
| | HGKTRWTQPQLICTG | |
| 29 | APTSSSTKKTQLQLEHLLLDLQMIL | hIL-2 |
| | NGINNYKNPKLTRMLTFKFYMPKKA | |
| | TELKHLQCLEEELKPLEEVLNLAQS | |
| | KNFHLRPRDLISNINVIVLELKGSE | |
| | TTFMCEYADETATIVEFLNRWITFC | |
| | QSIISTLT | |
| 30 | GGGGSGGGGSGGGGS | Linker |
| 31 | GGGGGGGGSGGGGSGGGGSGGGGS | Linker |
| 32 | GGGGS | Linker |

### Example 2: In Vivo Anti-Tumor Efficacy of the Combination Therapy of MAGE-A4 TCR-T Cells+REGN10597

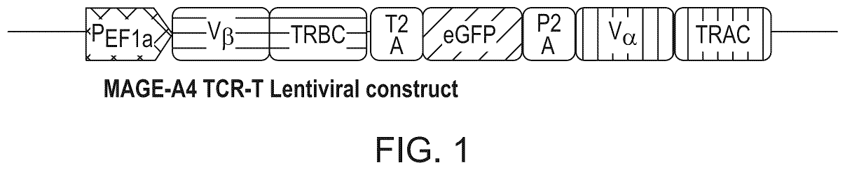

Generation of TCR-T cells: A human TCR (derived from a VelociT mouse) targeting HLA-A2/MAGE-A4₂₃₀₋₂₃₉ (PN45545) (WO 2020/257288) was cloned into a pLVX lentiviral vector with an EF1a promoter and T2A:eGFP sequence to facilitate tracking of transduced T cells. VSV-pseudotyped lentivirus was produced for transduction of primary human T cells (FIG. 1). Table 3 sets forth the amino acid sequences of an example MAGE-A4 TCR-T lentiviral construct.

Amino acid sequences of an example
MAGE-A4 TCR-T lentiviral construct

| SEQ | | INFORMA- |
|---|---|---|
| ID NO | SEQUENCE | TION |
| 33 | ATGGGAATTCGCTTGCTCTGTCGCG | Vb |
| | TCGCTTTCTGTTTTCTCGCCGTCGG | |
| | ACTTGTGGATGTCAAGGTCACCCAG | |
| | TCCTCCCGCTACCTGGTCAAGAGGA | |
| | CTGGAGAGAAAGTGTTCCTGGAATG | |
| | CGTGCAGGACATGGACCATGAAAAC | |
| | ATGTTCTGGTATAGACAGGACCCCG | |
| | GGCTGGGACTGCGGCTGATCTACTT | |
| | CTCCTACGACGTGAAGATGAAGGAA | |
| | AAGGGCGACATCCCTGAGGGATACT | |
| | CAGTGTCAAGAGAGAAGAAGGAGCG | |
| | GTTCTCCCTTATCCTGGAATCCGCC | |
| | TCGACTAATCAGACCTCGATGTACC | |
| | TGTGCGCGTCCTCCTTTACCGGTCC | |
| | TTACAACTCCCCCCTGCACTTCGGG | |
| | AATGGCACCCGGCTGACTGTGACC | |
| 34 | GAAGATCTCAACAAAGTGTTTCCTC | TRBC |
| | CGGAAGTGGCAGTCTTCGAGCCATC | |
| | CGAAGCCGAGATCAGCCACACTCAG | |
| | AAGGCCACCCTGGTCTGCTTGGCTA | |
| | CCGGATTCTTCCCTGACCACGTGGA | |
| | ACTTTCTTGGTGGGTGAACGGAAAA | |
| | GAAGTCCACTCCGGAGTCTCCACTG | |
| | ACCCTCAGCCGCTGAAGGAACAGCC | |
| | GGCCTTGAACGACTCGCGCTACTGC | |
| | CTGTCCTCCCGGCTGAGAGTGTCCG | |
| | CCACGTTCTGGCAAAACCCGAGGAA | |
| | CCATTTCCGGTGCCAAGTGCAGTTC | |
| | TACGGACTCAGCGAGAACGACGAGT | |
| | GGACCCAGGACAGGGCAAAGCCCGT | |
| | GACTCAAATCGTGTCCGCCGAAGCC | |
| | TGGGGACGGGCTGATTGCGGCTTCA | |
| | CCAGCGTGTCATATCAGCAAGGAGT | |
| | GCTGTCGGCCACTATCCTCTACGAG | |
| | ATTCTCTTGGGCAAAGCAACACTGT | |
| | ACGCGGTGCTCGTCAGCGCCCTGGT | |
| | GCTGATGGCCATGGTCAAGCGCAAG | |
| | GACTTT | |
| 35 | GGATCCGGA | GSG |
| 36 | GAGGGCAGAGGAAGTCTTCTAACAT | T2A |
| | GCGGTGACGTGGAGGAGAATCCCGG | |
| | CCCT | |
| 37 | ATGGTGAGCAAGGGAGAGGAGCTGT | eGFP |
| | TCACCGGAGTGGTGCCAATCCTGGT | |
| | GGAGCTGGACGGCGATGTGAATGGC | |
| | CACAAGTTTAGCGTGTCCGGAGAGG | |
| | GAGAGGGCGACGCAACATACGGCAA | |
| | GCTGACCCTGAAGTTCATCTGCACA | |
| | ACCGGCAAGCTGCCTGTGCCATGGC | |
| | CCACACTGGTGACAACCCTGACCTA | |
| | CGGCGTGCAGTGTTTCTCTAGATAT | |
| | CCAGATCACATGAAGCAGCACGACT | |
| | TCTTTAAGAGCGCCATGCCAGAGGG | |
| | ATACGTGCAGGAGCGCACCATCTTC | |
| | TTTAAGGACGATGGCAACTATAAGA | |
| | CACGGGCCGAGGTGAAGTTCGAGGG | |
| | CGATACCCTGGTGAACAGAATCGAG | |
| | CTGAAGGGCATCGACTTCAAGGAGG | |
| | ACGGCAATATCCTGGGCCACAAGCT | |
| | GGAGTACAACTATAATAGCCACAAC | |
| | GTGTACATCATGGCCGACAAGCAGA | |
| | AGAACGGCATCAAGGTGAACTTCAA | |
| | GATCCGGCACAATATCGAGGATGGC | |
| | TCCGTGCAGCTGGCCGACCACTACC | |
| | AGCAGAACACACCAATCGGCGATGG | |
| | CCCAGTGCTGCTGCCCGACAATCAC | |
| | TATCTGTCTACCCAGAGCGCCCTGT | |
| | CCAAGGATCCCAACGAGAAGAGAGA | |
| | CCACATGGTGCTGCTGGAGTTCGTG | |
| | ACAGCAGCAGGAATCACCCTGGGAA | |
| | TGGACGAGCTGTATAAG | |
| 38 | CGGGCCAAGCGC | Furin |
| 39 | GCGACTAACTTTTCCCTGCTGAAGC | P2A |
| | AGGCTGGCGATGTGGAAGAGAACCC | |
| | TGGGCCA | |
| 40 | ATGTCCCTGAGCAGCCTGCTGAAGG | Va |
| | TCGTGACCGCGTCATTGTGGCTGGG | |
| | ACCGGGCATTGCCCAGAAGATCACC | |
| | CAGACCCAGCCGGGGATGTTTGTGC | |
| | AAGAAAAGGAAGCCGTTACCCTCGA | |
| | CTGCACTTACGACACCTCCGACCCG | |
| | TCATACGGACTGTTCTGGTACAAGC | |
| | AACCCAGCAGCGGAGAAATGATCTT | |
| | CCTGATCTACCAAGGGTCCTACGAC | |
| | CAGCAGAATGCTACCGAAGGTCGCT | |
| | ACAGCCTGAATTTCCAGAAGGCCCG | |
| | CAAGAGCGCCAACCTCGTGATTTCT | |
| | GCCTCCCAACTCGGCGATTCCGCAA | |
| | TGTACTTCTGTGCGATGCGGGGTGG | |
| | CGGCTCCGGCGGCAGCTACATCCCC | |
| | ACCTTCGGTCGGGGCACCTCACTGA | |
| | TTGTGCACCCA | |
| 41 | TACATCCAGAATCCGGATCCTGCGG | |
| | TCTATCAATTAAGGGACTCCAAGTC | |
| | TTCCGATAAATCCGTGTGTCTCTTT | |
| | ACAGACTTCGACTCGCAAACCAACG | |
| | TGTCCCAGTCAAAGGACTCGGATGT | |
| | GTACATCACCGACAAGACTGTGCTG | |
| | GACATGCGGTCGATGGACTTCAAGT | |
| | CCAACAGCGCGGTGGCCTGGTCCAA | |
| | CAAGAGCGACTTCGCCTGTGCGAAC | |
| | GCCTTCAACAACTCCATCATTCCCG | |
| | AGGACACCTTCTTCCCATCCCCTGA | |
| | GTCCTCCTGCGACGTGAAGCTCGTG | |
| | GAGAAGTCGTTCGAGACTGATACCA | |
| | ACCTGAACTTTCAAAACCTGAGCGT | |
| | GATAGGGTTCAGGATCCTGTTACTC | |
| | AAAGTCGCCGGTTTCAACCTCCTGA | |
| | TGACCCTGAGACTTTGGTCAAGT | |
| 42 | ATGGGAATTCGCTTGCTCTGTCGCG | TRAC |
| | TCGCTTTCTGTTTTCTCGCCGTCGG | |
| | ACTTGTGGATGTCAAGGTCACCCAG | |
| | TCCTCCCGCTACCTGGTCAAGAGGA | |
| | CTGGAGAGAAAGTGTTCCTGGAATG | |
| | CGTGCAGGACATGGACCATGAAAAC | |
| | ATGTTCTGGTATAGACAGGACCCCG | |
| | GGCTGGGACTGCGGCTGATCTACTT | |
| | CTCCTACGACGTGAAGATGAAGGAA | |
| | AAGGGCGACATCCCTGAGGGATACT | |
| | CAGTGTCAAGAGAGAAGAAGGAGCG | |
| | GTTCTCCCTTATCCTGGAATCCGCC | |
| | TCGACTAATCAGACCTCGATGTACC | |
| | TGTGCGCGTCCTCCTTTACCGGTCC | |
| | TTACAACTCCCCCCTGCACTTCGGG | |
| | AATGGCACCCGGCTGACTGTGACCG | |
| | AAGATCTCAACAAAGTGTTTCCTCC | |
| | GGAAGTGGCAGTCTTCGAGCCATCC | |
| | GAAGCCGAGATCAGCCACACTCAGA | |
| | AGGCCACCCTGGTCTGCTTGGCTAC | |
| | CGGATTCTTCCCTGACCACGTGGAA | |
| | CTTTCTTGGTGGGTGAACGGAAAAG | |
| | AAGTCCACTCCGGAGTCTCCACTGA | |
| | CCCTCAGCCGCTGAAGGAACAGCCG | |
| | GCCTTGAACGACTCGCGCTACTGCC | |
| | TGTCCTCCCGGCTGAGAGTGTCCGC | |
| | CACGTTCTGGCAAAACCCGAGGAAC | |
| | CATTTCCGGTGCCAAGTGCAGTTCT | |
| | ACGGACTCAGCGAGAACGACGAGTG | |
| | GACCCAGGACAGGGCAAAGCCCGTG | |
| | ACTCAAATCGTGTCCGCCGAAGCCT | |
| | GGGGACGGGCTGATTGCGGCTTCAC | |
| | CAGCGTGTCATATCAGCAAGGAGTG | |
| | CTGTCGGCCACTATCCTCTACGAGA | |
| | TTCTCTTGGGCAAAGCAACACTGTA | |
| | CGCGGTGCTCGTCAGCGCCCTGGTG | |
| | CTGATGGCCATGGTCAAGCGCAAGG | |
| | ACTTTGGATCCGGAGAGGGCAGAGG | |
| | AAGTCTTCTAACATGCGGTGACGTG | |
| | GAGGAGAATCCCGGCCCTATGGTGA | |
| | GCAAGGGAGAGGAGCTGTTCACCGG | |
| | AGTGGTGCCAATCCTGGTGGAGCTG | |
| | GACGGCGATGTGAATGGCCACAAGT | |
| | TTAGCGTGTCCGGAGAGGGAGAGGG | |
| | CGACGCAACATACGGCAAGCTGACC | |
| | CTGAAGTTCATCTGCACAACCGGCA | |
| | AGCTGCCTGTGCCATGGCCCACACT | |
| | GGTGACAACCCTGACCTACGGCGTG | |
| | CAGTGTTTCTCTAGATATCCAGATC | |
| | ACATGAAGCAGCACGACTTCTTTAA | |
| | GAGCGCCATGCCAGAGGGATACGTG | |
| | CAGGAGCGCACCATCTTCTTTAAGG | |
| | ACGATGGCAACTATAAGACACGGGC | |
| | CGAGGTGAAGTTCGAGGGCGATACC | |
| | CTGGTGAACAGAATCGAGCTGAAGG | |
| | GCATCGACTTCAAGGAGGACGGCAA | |
| | TATCCTGGGCCACAAGCTGGAGTAC | |
| | AACTATAATAGCCACAACGTGTACA | |
| | TCATGGCCGACAAGCAGAAGAACGG | |
| | CATCAAGGTGAACTTCAAGATCCGG | |
| | CACAATATCGAGGATGGCTCCGTGC | |
| | AGCTGGCCGACCACTACCAGCAGAA | |
| | CACACCAATCGGCGATGGCCCAGTG | |
| | CTGCTGCCCGACAATCACTATCTGT | |
| | CTACCCAGAGCGCCCTGTCCAAGGA | |
| | TCCCAACGAGAAGAGAGACCACATG | |
| | GTGCTGCTGGAGTTCGTGACAGCAG | |
| | CAGGAATCACCCTGGGAATGGACGA | |
| | GCTGTATAAGCGGGCCAAGCGCGGA | |
| | TCCGGAGCGACTAACTTTTCCCTGC | |
| | TGAAGCAGGCTGGCGATGTGGAAGA | |
| | GAACCCTGGGCCAATGTCCCTGAGC | |
| | AGCCTGCTGAAGGTCGTGACCGCGT | |
| | CATTGTGGCTGGGACCGGGCATTGC | |
| | CCAGAAGATCACCCAGACCCAGCCG | |
| | GGGATGTTTGTGCAAGAAAAGGAAG | |
| | CCGTTACCCTCGACTGCACTTACGA | |
| | CACCTCCGACCCGTCATACGGACTG | |
| | TTCTGGTACAAGCAACCCAGCAGCG | |
| | GAGAAATGATCTTCCTGATCTACCA | |
| | AGGGTCCTACGACCAGCAGAATGCT | |
| | ACCGAAGGTCGCTACAGCCTGAATT | |
| | TCCAGAAGGCCCGCAAGAGCGCCAA | |
| | CCTCGTGATTTCTGCCTCCCAACTC | |
| | GGCGATTCCGCAATGTACTTCTGTG | |
| | CGATGCGGGGTGGCGGCTCCGGCGG | |
| | CAGCTACATCCCCACCTTCGGTCGG | |
| | GGCACCTCACTGATTGTGCACCCAT | |
| | ACATCCAGAATCCGGATCCTGCGGT | |
| | CTATCAATTAAGGGACTCCAAGTCT | |
| | TCCGATAAATCCGTGTGTCTCTTTA | |
| | CAGACTTCGACTCGCAAACCAACGT | |
| | GTCCCAGTCAAAGGACTCGGATGTG | |
| | TACATCACCGACAAGACTGTGCTGG | |
| | ACATGCGGTCGATGGACTTCAAGTC | |
| | CAACAGCGCGGTGGCCTGGTCCAAC | |
| | AAGAGCGACTTCGCCTGTGCGAACG | |
| | CCTTCAACAACTCCATCATTCCCGA | |
| | GGACACCTTCTTCCCATCCCCTGAG | |
| | TCCTCCTGCGACGTGAAGCTCGTGG | |
| | AGAAGTCGTTCGAGACTGATACCAA | |
| | CCTGAACTTTCAAAACCTGAGCGTG | |
| | ATAGGGTTCAGGATCCTGTTACTCA | |
| | AAGTCGCCGGTTTCAACCTCCTGAT | |
| | GACCCTGAGACTTTGGTCAAGT | |

CD3+ T cells were isolated from human peripheral blood mononuclear cells (PBMCs) and stimulated with CD3/CD28 microbeads plus 100 U/ml recombinant human IL-2. On Day 3 after stimulation, endogenous TCRs were deleted via CRISPR/Cas9 targeting, followed by transduction with the lentivirus at a MOI=5. The transduced cells were expanded for 14 days with CD3/CD28 microbeads plus 100 U/ml recombinant human IL-2 before cryopreservation until the in vivo experiment.

Implantation and Measurement of Xenogeneic Tumors

On day −10, immunodeficient NOD.Cg-Prkdc^{scid}Il2rg^{tm1Wjl}/SzJ (NSG) mice were subcutaneously injected with 5×10⁶ HLA-A2+MAGEA4+A375 human melanoma tumor cells. Using mass spectrometry techniques, it was determined that A375 melanoma cells express approximately 450 cell-surface copies of the MAGEA4₂₃₀₋₂₃₉ peptide. On day 0 (10 days after tumor implantation), mice were randomized and intravenously injected with MAGE-A4 TCR-T at 3 dose levels: 4.0×10⁶, 2.0×10⁶, or 1.0×10⁶ MAGE-A4₂₃₀₋₂₃₉ tetramer-positive TCR-T cells. Control groups received 4.0×10⁶ irrelevant tetramer-positive TCR-T (Control TCR-T). REGN10597 (0.5 mg/kg) was administered intraperitoneally on days 7, 14, and 21 after T cell dosing. A non-targeted control anti-MUC16-IL2Ra-IL2 (REGN9903) was administered as isotype control. Tumor growth was assessed for up to 49 days post-T cell dose. Mice were euthanized when tumor diameter exceeded 20 mm, in accordance with IACUC protocols.

Calculation of Xenogenic Tumor Growth and Inhibition

To determine tumor volume by external caliper, the greatest longitudinal diameter (length in mm) and the greatest transverse diameter (width in mm) were determined. Tumor volumes based on caliper measurements were calculated by the formula: Volume (mm³)=(length×width²)/2.

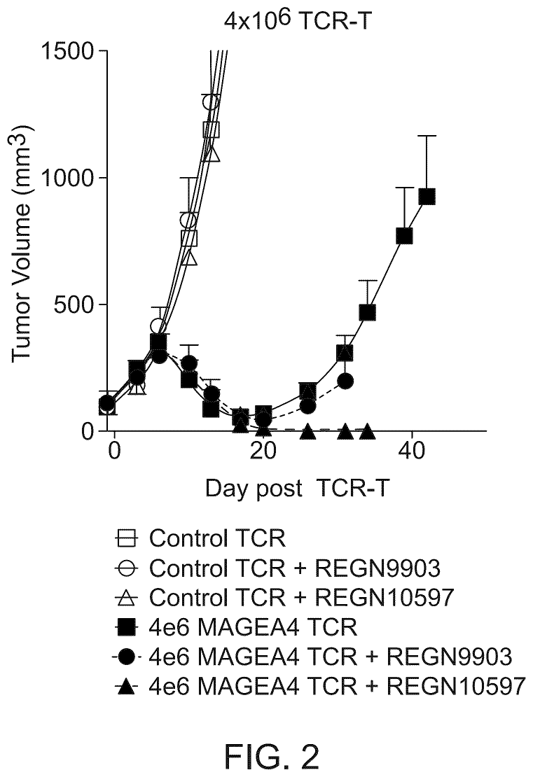

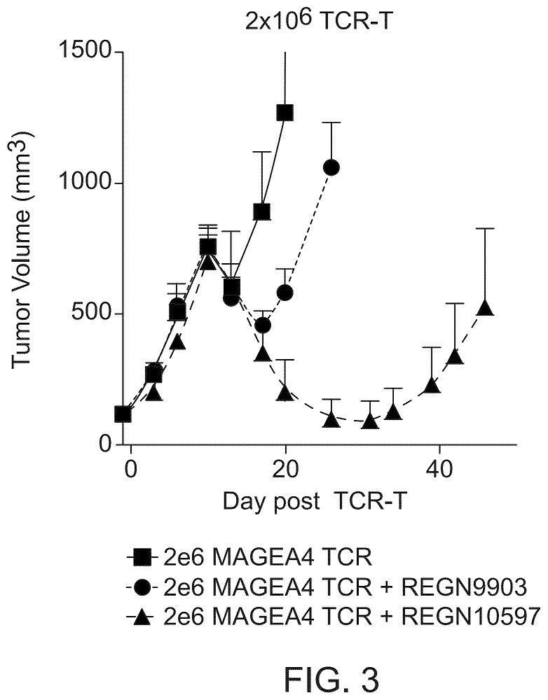

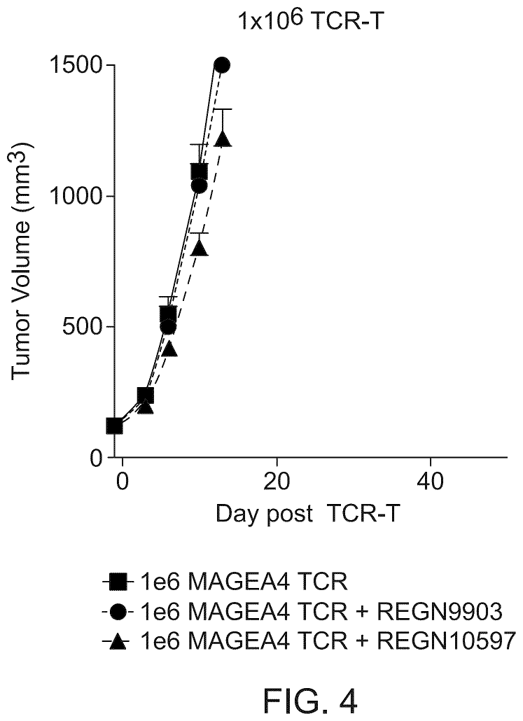

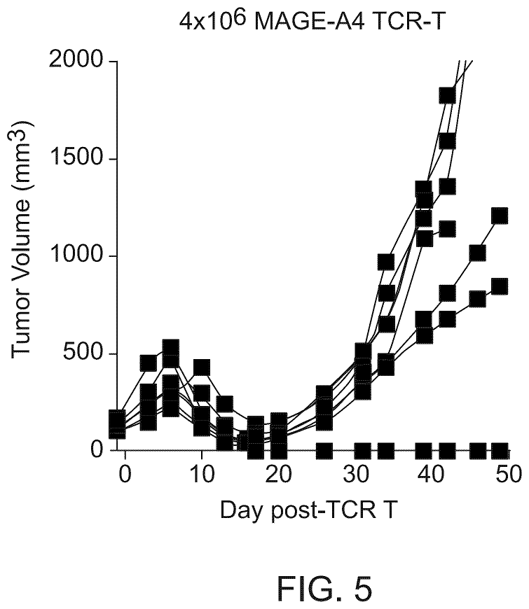

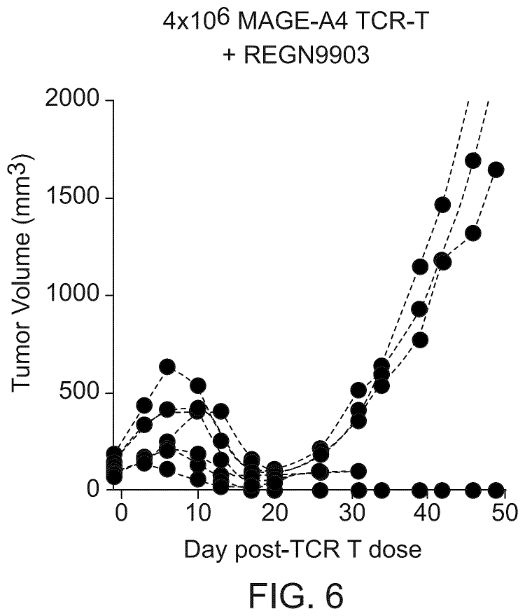

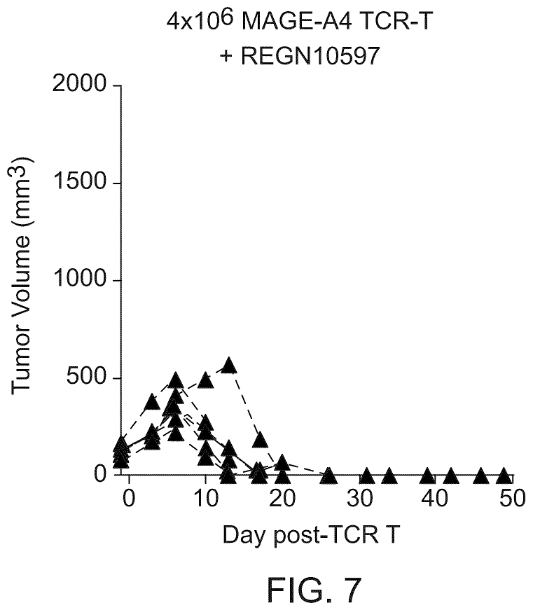

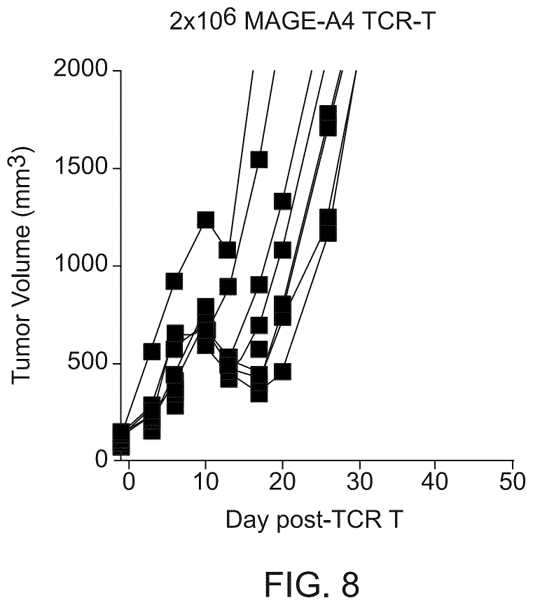

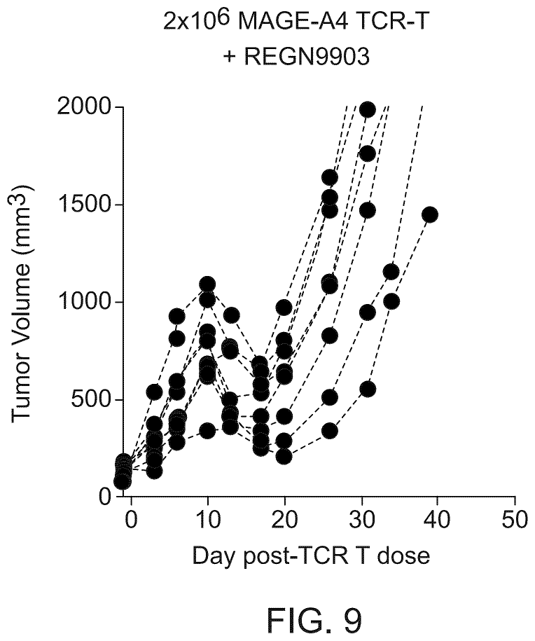

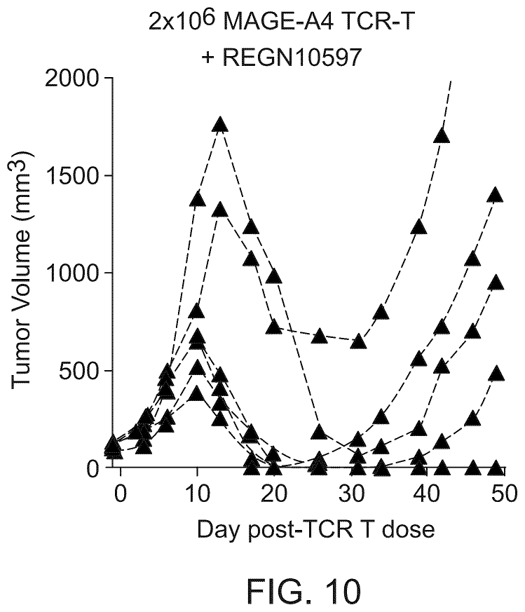

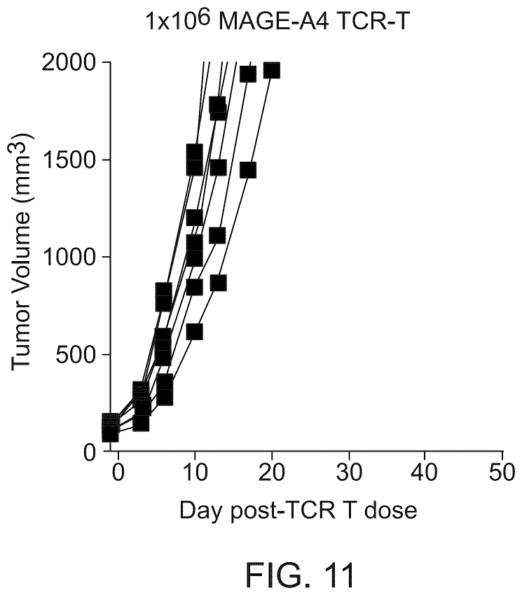

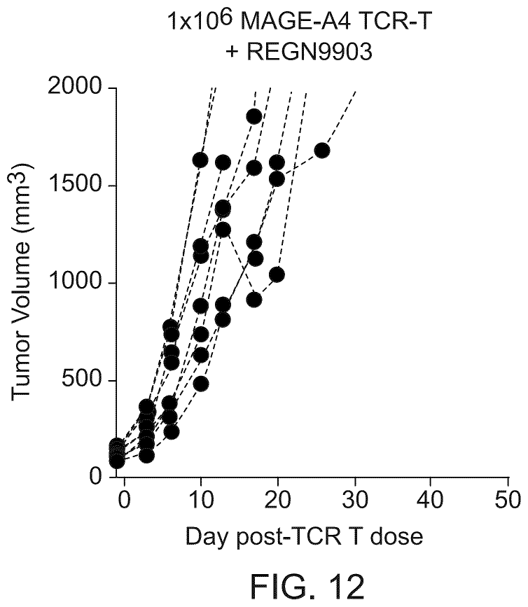

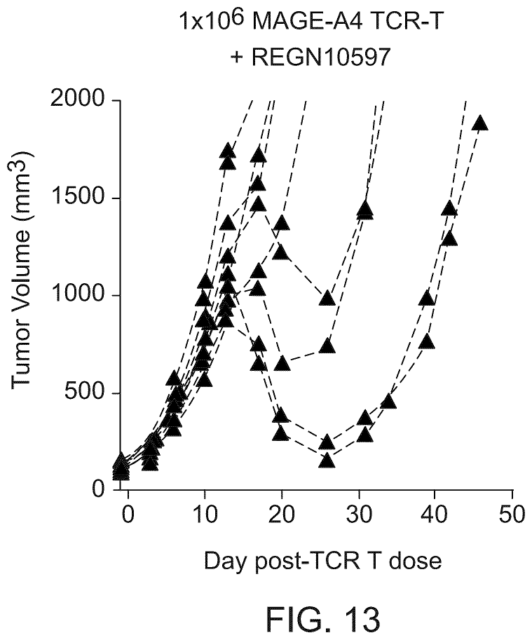

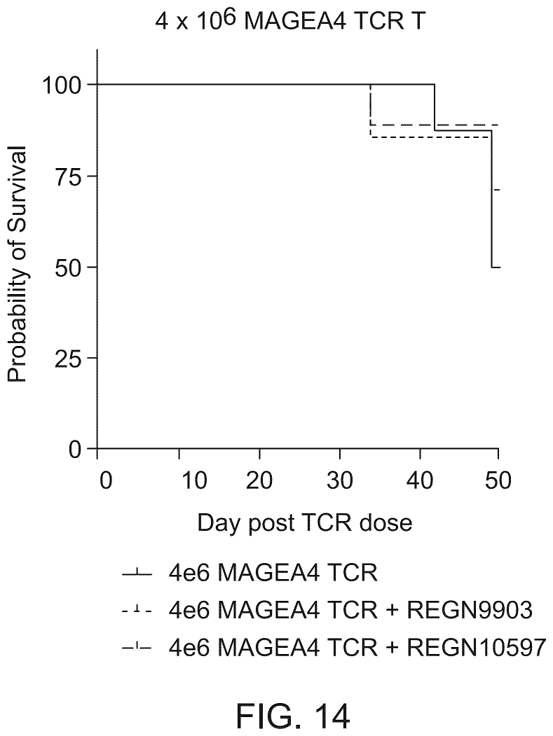

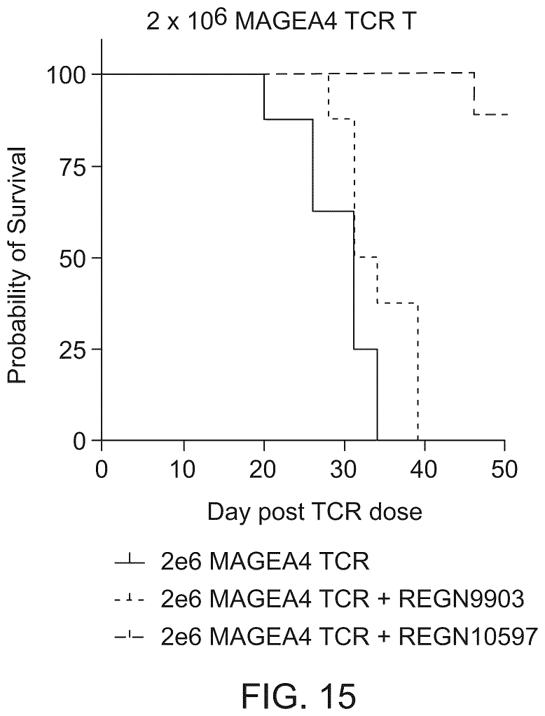

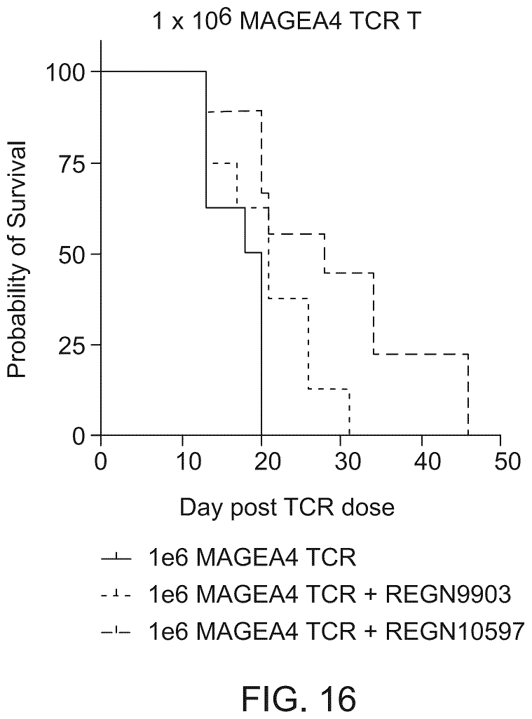

A375 tumors grew progressively in mice receiving no treatment or irrelevant control TCR-T (FIG. 2; Tables 4-16). MAGE-A4 TCR-T monotherapy demonstrated dose-dependent anti-tumor activity (FIGS. 2-4; Tables 4-16). The addition of 0.5 mg/kg of REGN10597 beginning 7 days after T cell dosing augmented anti-tumor activity at each dose level (FIGS. 2-16; Tables 4-17). 4×10⁶ MAGE-A4 TCR-T alone induced initial tumor regressions that were short-lived, with most tumors recurring within 1 month of dosing (2 of 8 mice tumor-free on day 31) (Table 11). The addition of REGN10597 significantly enhanced tumor control, and 8 out of 9 mice remained tumor-free for the remainder of the study. One mouse receiving 4×10⁶ MAGE-A4 TCR T+REGN10597 was euthanized on day 34 due to weight loss; there was no indication that this death was treatment-related. Similarly, 2×10⁶ MAGE-A4 TCR-T alone demonstrated very modest and transient anti-tumor activity which was significantly enhanced by REGN10597 (6 of 9 mice tumor-free on day 20) (Table 9). Augmented tumor control is also reflected in significantly increased probability of survival of mice treated with 2×10⁶ MAGE-A4 TCR-T+REGN10597 compared to animals receiving MAGE-A4 TCR-T alone or REGN9903 p=<0.0001 (Log-rank (Mantel-Cox) test)). Lastly, 1×10⁶ MAGE-A4 TCR-T alone showed no difference in tumor growth compared to control-treated animals, but the combination of 1×10⁶ MAGE-A4 TCR-T with REGN10597 significantly delayed tumor growth (p=0.023) and significantly enhanced survival compared to 1×10⁶ MAGE-A4 TCR-T alone p=0.0051 (Log-rank (Mantel-Cox) test) (FIGS. 4, 13, and 16). Neither irrelevant TCR-T+REGN10597 nor MAGE-A4 TCR-T+non-targeted IL2Ra-IL2 REGN9903 mediated any additional effects on anti-tumor efficacy (FIG. 2).

Collectively these data show that REGN10597 augments the in vivo anti-tumor activity of engineered human MAGE-A4 TCR-T cells. Accordingly, this representative example supports the expectation that administration of ACT in combination with a targeted immunocytokine to a subject with cancer will lead to increased efficacy and duration of anti-tumor response, as compared to a subject treated with the ACT as monotherapy.

TABLE 4
Treatment effects on Day 3 of a combination therapy
of MAGE-A4 TCR-T cells + REGN10597 anti-PD1-IL2Ra-IL2

| | | Tumor size | Number of |
|---|---|---|---|
| | Average | standard | mice still |
| | tumor size | error of the | alive on |
| Treatment | on Day 3 | mean (SEM) | Day 3 |
| None (n = 5) | 125.2 | 15.2 | 5 |
| 4.0 × 10⁶ Control TCR-T (n = 5) | 488.4 | 21.5 | 5 |
| 4.0 × 10⁶ Control TCR-T + REGN9903 (n = 5) | 216.2 | 40.7 | 5 |
| 4.0 × 10⁶ Control TCR-T + REGN10597 (n = 5) | 178.9 | 6.8 | 5 |
| 4.0 × 10⁶ MAGE-A4 TCR-T (n = 8) | 241.0 | 33.5 | 8 |
| 4.0 × 10⁶ MAGE-A4 TCR-T + REGN9903 (n = 7) | 224.0 | 44.4 | 7 |
| 4.0 × 10⁶ MAGE-A4 TCR-T + REGN10597 (n = 9) | 223.6 | 21.5 | 9 |
| 2.0 × 10⁶ MAGE-A4 TCR-T (n = 8) | 269.2 | 40.1 | 8 |
| 2.0 × 10⁶ MAGE-A4 TCR-T + REGN9903 (n = 8) | 278.3 | 46.4 | 8 |
| 2.0 × 10⁶ MAGE-A4 TCR-T + REGN10597 (n = 9) | 203.8 | 16.2 | 9 |
| 1.0 × 10⁶ MAGE-A4 TCR-T (n = 8) | 240.2 | 22.7 | 8 |
| 1.0 × 10⁶ MAGE-A4 TCR-T + REGN9903 (n = 8) | 228.0 | 32.7 | 8 |
| 1.0 × 10⁶ MAGE-A4 TCR-T + REGN10597 (n = 9) | 202.3 | 12.2 | 9 |

TABLE 5
Treatment effects on Day 6 of a combination therapy
of MAGE-A4 TCR-T cells + REGN10597 anti-PD1-IL2Ra-IL2

| | | Tumor size | Number |
|---|---|---|---|
| | Average | standard | of mice |
| | tumor size | error of the | still alive |
| Treatment | on Day 6 | mean (SEM) | on Day 6 |
| None (n = 5) | 206.1 | 26.2 | 5 |
| 4.0 × 10⁶ Control TCR-T (n = 5) | 375.9 | 40.3 | 5 |
| 4.0 × 10⁶ Control TCR-T + REGN9903 (n = 5) | 408.2 | 79.9 | 5 |
| 4.0 × 10⁶ Control TCR-T + REGN10597 (n = 5) | 316.2 | 17.8 | 5 |
| 4.0 × 10⁶ MAGE-A4 TCR-T (n = 8) | 347.5 | 37.7 | 8 |
| 4.0 × 10⁶ MAGE-A4 TCR-T + REGN9903 (n = 7) | 296.3 | 65.4 | 7 |
| 4.0 × 10⁶ MAGE-A4 TCR-T + REGN10597 (n = 9) | 329.4 | 29.5 | 9 |
| 2.0 × 10⁶ MAGE-A4 TCR-T (n = 8) | 504.8 | 72.6 | 8 |
| 2.0 × 10⁶ MAGE-A4 TCR-T + REGN9903 (n = 8) | 533.5 | 82.9 | 8 |
| 2.0 × 10⁶ MAGE-A4 TCR-T + REGN10597 (n = 9) | 397.4 | 32.2 | 9 |
| 1.0 × 10⁶ MAGE-A4 TCR-T (n = 8) | 548.7 | 65.7 | 8 |
| 1.0 × 10⁶ MAGE-A4 TCR-T + REGN9903 (n = 8) | 500.0 | 65.7 | 8 |
| 1.0 × 10⁶ MAGE-A4 TCR-T + REGN10597 (n = 9) | 420.7 | 29.9 | 9 |

TABLE 6
Treatment effects on Day 10 of combination therapy of
MAGE-A4 TCR-T cells + REGN10597 anti-PD1-IL2Ra-IL2

| | | Tumor size | Number of |
|---|---|---|---|
| | Average | standard | mice still |
| | tumor size | error of the | alive on |
| Treatment | on Day 10 | mean (SEM) | Day 10 |
| None (n = 5) | 481.9 | 35.9 | 5 |
| 4.0 × 10⁶ Control TCR-T (n = 5) | 763.7 | 97.1 | 5 |
| 4.0 × 10⁶ Control TCR-T + REGN9903 (n = 5) | 834.2 | 164.3 | 5 |
| 4.0 × 10⁶ Control TCR-T + REGN10597 (n = 5) | 686.7 | 34.4 | 5 |
| 4.0 × 10⁶ MAGE-A4 TCR-T (n = 8) | 200.3 | 38.2 | 8 |
| 4.0 × 10⁶ MAGE-A4 TCR-T + REGN9903 (n = 7) | 269.3 | 69.8 | 7 |
| 4.0 × 10⁶ MAGE-A4 TCR-T + REGN10597 (n = 9) | 244.1 | 37.0 | 9 |
| 2.0 × 10⁶ MAGE-A4 TCR-T (n = 8) | 756.4 | 71.6 | 8 |
| 2.0 × 10⁶ MAGE-A4 TCR-T + REGN9903 (n = 8) | 755.0 | 83.7 | 8 |
| 2.0 × 10⁶ MAGE-A4 TCR-T + REGN10597 (n = 9) | 703.1 | 95.0 | 9 |
| 1.0 × 10⁶ MAGE-A4 TCR-T (n = 8) | 1093.4 | 107.0 | 8 |
| 1.0 × 10⁶ MAGE-A4 TCR-T + REGN9903 (n = 8) | 1040.1 | 154.6 | 8 |
| 1.0 × 10⁶ MAGE-A4 TCR-T + REGN10597 (n = 9) | 804.4 | 52.4 | 9 |

TABLE 7
Treatment effects on Day 13 of a combination therapy
of MAGE-A4 TCR-T cells + REGN10597 anti-PD1-IL2Ra-IL2

| | | Tumor size | Number of |
|---|---|---|---|
| | Average | standard | mice still |
| | tumor size | error of the | alive on |
| Treatment | on Day 13 | mean (SEM) | Day 13 |
| None (n = 5) | 821.3 | 29.1 | 5 |
| 4.0 × 10⁶ Control TCR-T (n = 5) | 1187.0 | 140.4 | 5 |
| 4.0 × 10⁶ Control TCR-T + REGN9903 (n = 5) | 1300.4 | 213.4 | 5 |
| 4.0 × 10⁶ Control TCR-T + REGN10597 (n = 5) | 1100.5 | 113.9 | 5 |
| 4.0 × 10⁶ MAGE-A4 TCR-T (n = 8) | 88.2 | 23.1 | 8 |
| 4.0 × 10⁶ MAGE-A4 TCR-T + REGN9903 (n = 7) | 146.1 | 53.5 | 7 |
| 4.0 × 10⁶ MAGE-A4 TCR-T + REGN10597 (n = 9) | 101.0 | 61.6 | 9 |
| 2.0 × 10⁶ MAGE-A4 TCR-T (n = 8) | 602.6 | 86.2 | 8 |
| 2.0 × 10⁶ MAGE-A4 TCR-T + REGN9903 (n = 8) | 564.7 | 76.5 | 8 |
| 2.0 × 10⁶ MAGE-A4 TCR-T + REGN10597 (n = 9) | 642.9 | 177.2 | 9 |
| 1.0 × 10⁶ MAGE-A4 TCR-T (n = 8) | 1717.9 | 209.3 | 8 |
| 1.0 × 10⁶ MAGE-A4 TCR-T + REGN9903 (n = 8) | 1497.3 | 199.1 | 8 |
| 1.0 × 10⁶ MAGE-A4 TCR-T + REGN10597 (n = 9) | 1222.0 | 106.9 | 9 |

TABLE 8
Treatment effects on Day 17 of a combination therapy
of MAGE-A4 TCR-T cells + REGN10597 anti-PD1-IL2Ra-IL2

| | | Tumor size | Number of |
|---|---|---|---|
| | Average | standard | mice still |
| | tumor size | error of the | alive on |
| Treatment | on Day 17 | mean (SEM) | on Day 17 |
| None (n = 5) | 1387.7 | 37.6 | 5 |
| 4.0 × 10⁶ Control TCR-T (n = 5) | 2014.1 | 318.6 | 5 |
| 4.0 × 10⁶ Control TCR-T + REGN9903 (n = 5) | 2220.1 | 397.0 | 5 |
| 4.0 × 10⁶ Control TCR-T + REGN10597 (n = 5) | 1852.1 | 156.2 | 5 |
| 4.0 × 10⁶ MAGE-A4 TCR-T (n = 8) | 55.3 | 16.0 | 8 |
| 4.0 × 10⁶ MAGE-A4 TCR-T + REGN9903 (n = 7) | 52.1 | 24.0 | 7 |
| 4.0 × 10⁶ MAGE-A4 TCR-T + REGN10597 (n = 9) | 24.5 | 21.0 | 9 |
| 2.0 × 10⁶ MAGE-A4 TCR-T (n = 8) | 892.5 | 233.2 | 8 |
| 2.0 × 10⁶ MAGE-A4 TCR-T + REGN9903 (n = 8) | 457.2 | 61.1 | 8 |
| 2.0 × 10⁶ MAGE-A4 TCR-T + REGN10597 (n = 9) | 354.7 | 155.6 | 9 |
| 1.0 × 10⁶ MAGE-A4 TCR-T (n = 8) | 2296.8 | 287.4 | 5 |
| 1.0 × 10⁶ MAGE-A4 TCR-T + REGN9903 (n = 8) | 1760.5 | 441.6 | 6 |
| 1.0 × 10⁶ MAGE-A4 TCR-T + REGN10597 (n = 9) | 1311.4 | 176.2 | 8 |

TABLE 9
Treatment effects on Day 20 of a combination therapy
of MAGE-A4 TCR-T cells + REGN10597 anti-PD1-IL2Ra-IL2

| | | Tumor size | Number of |
|---|---|---|---|
| | Average | standard | mice still |
| | tumor size | error of the | alive on |
| Treatment | on Day 20 | mean (SEM) | Day 20 |
| None (n = 5) | 1896.4 | 128.1 | 5 |
| 4.0 × 10⁶ Control TCR-T (n = 5) | 2083.5 | 209.4 | 3 |
| 4.0 × 10⁶ Control TCR-T + REGN9903 (n = 5) | 1858.4 | 379.0 | 2 |
| 4.0 × 10⁶ Control TCR-T + REGN10597 (n = 5) | 2418.5 | 104.2 | 2 |
| 4.0 × 10⁶ MAGE-A4 TCR-T (n = 8) | 68.3 | 17.7 | 8 |
| 4.0 × 10⁶ MAGE-A4 TCR-T + REGN9903 (n = 7) | 42.7 | 16.7 | 7 |
| 4.0 × 10⁶ MAGE-A4 TCR-T + REGN10597 (n = 9) | 7.6 | 7.6 | 9 |
| 2.0 × 10⁶ MAGE-A4 TCR-T (n = 8) | 1269.2 | 291.7 | 8 |
| 2.0 × 10⁶ MAGE-A4 TCR-T + REGN9903 (n = 8) | 581.3 | 93.6 | 8 |
| 2.0 × 10⁶ MAGE-A4 TCR-T + REGN10597 (n = 9) | 200.4 | 127.3 | 9 |
| 1.0 × 10⁶ MAGE-A4 TCR-T (n = 8) | 3059.7 | 399.6 | 4 |
| 1.0 × 10⁶ MAGE-A4 TCR-T + REGN9903 (n = 8) | 2008.8 | 450.4 | 5 |
| 1.0 × 10⁶ MAGE-A4 TCR-T + REGN10597 (n = 9) | 1348.9 | 300.1 | 8 |

TABLE 10
Treatment effects on Day 26 of a combination therapy
of MAGE-A4 TCR-T cells + REGN10597 anti-PD1-IL2Ra-IL2

| | | Tumor size | Number of |
|---|---|---|---|
| | Average | standard | mice still |
| | tumor size | error of the | alive on |
| Treatment | on Day 26 | mean (SEM) | Day 26 |
| None (n = 5) | 2801.5 | 91.5 | 2 |
| 4.0 × 10⁶ Control TCR-T (n = 5) | 2882.7 | 444.1 | 2 |
| 4.0 × 10⁶ Control TCR-T + REGN9903 (n = 5) | 2341.4 | | 1 |
| 4.0 × 10⁶ Control TCR-T + REGN10597 (n = 5) | | | 0 |
| 4.0 × 10⁶ MAGE-A4 TCR-T (n = 8) | 152.5 | 38.0 | 8 |
| 4.0 × 10⁶ MAGE-A4 TCR-T + REGN9903 (n = 7) | 98.6 | 37.5 | 7 |
| 4.0 × 10⁶ MAGE-A4 TCR-T + REGN10597 (n = 9) | 0.0 | 0.0 | 9 |
| 2.0 × 10⁶ MAGE-A4 TCR-T (n = 8) | 1885.1 | 224.7 | 7 |
| 2.0 × 10⁶ MAGE-A4 TCR-T + REGN9903 (n = 8) | 1062.2 | 170.4 | 8 |
| 2.0 × 10⁶ MAGE-A4 TCR-T + REGN10597 (n = 9) | 102.1 | 75.3 | 9 |
| 1.0 × 10⁶ MAGE-A4 TCR-T (n = 8) | | | 0 |
| 1.0 × 10⁶ MAGE-A4 TCR-T + REGN9903 (n = 8) | 2301.3 | 320.8 | 3 |
| 1.0 × 10⁶ MAGE-A4 TCR-T + REGN10597 (n = 9) | 937.0 | 426.3 | 5 |

TABLE 11
Treatment effects on Day 31 of a combination therapy
of MAGE-A4 TCR-T cells + REGN10597 anti-PD1-IL2Ra-IL2

| | | Tumor size | Number of |
|---|---|---|---|
| | Average | standard | mice still |
| | tumor size | error of the | alive on |
| Treatment | on Day 31 | mean (SEM) | Day 31 |
| None (n = 5) | | | 0 |
| 4.0 × 10⁶ Control TCR-T (n = 5) | | | 0 |
| 4.0 × 10⁶ Control TCR-T + REGN9903 (n = 5) | | | 0 |
| 4.0 × 10⁶ Control TCR-T + REGN10597 (n = 5) | | | 0 |
| 4.0 × 10⁶ MAGE-A4 TCR-T (n = 8) | 306.4 | 70.8 | 8 |
| 4.0 × 10⁶ MAGE-A4 TCR-T + REGN9903 (n = 7) | 198.9 | 84.7 | 7 |
| 4.0 × 10⁶ MAGE-A4 TCR-T + REGN10597 (n = 9) | 0.0 | 0.0 | 9 |
| 2.0 × 10⁶ MAGE-A4 TCR-T (n = 8) | 2605.1 | 181.5 | 8 |
| 2.0 × 10⁶ MAGE-A4 TCR-T + REGN9903 (n = 8) | 1660.1 | 277.4 | 8 |
| 2.0 × 10⁶ MAGE-A4 TCR-T + REGN10597 (n = 9) | 95.7 | 71.5 | 9 |
| 1.0 × 10⁶ MAGE-A4 TCR-T (n = 8) | | | 0 |
| 1.0 × 10⁶ MAGE-A4 TCR-T + REGN9903 (n = 8) | 2047.7 | | 1 |
| 1.0 × 10⁶ MAGE-A4 TCR-T + REGN10597 (n = 9) | 895.7 | 322.6 | 4 |

TABLE 12
Treatment effects on Day 34 of a combination therapy
of MAGE-A4 TCR-T cells + REGN10597 anti-PD1-IL2Ra-IL2

| | | Tumor size | Number of |
|---|---|---|---|
| | Average | standard | mice still |
| | tumor size | error of the | alive on |
| Treatment | on Day 34 | mean (SEM) | Day 34 |
| None (n = 5) | | | 0 |
| 4.0 × 10⁶ Control TCR-T (n = 5) | | | 0 |
| 4.0 × 10⁶ Control TCR-T + REGN9903 (n = 5) | 2341.4 | | 1 |
| 4.0 × 10⁶ Control TCR-T + REGN10597 (n = 5) | | | 0 |
| 4.0 × 10⁶ MAGE-A4 TCR-T (n = 8) | 469.3 | 123.1 | 8 |
| 4.0 × 10⁶ MAGE-A4 TCR-T + REGN9903 (n = 7) | 298.0 | 133.9 | 6 |
| 4.0 × 10⁶ MAGE-A4 TCR-T + REGN10597 (n = 9) | 0.0 | 0.0 | 8 |
| 2.0 × 10⁶ MAGE-A4 TCR-T (n = 8) | 3099.5 | 146.2 | 8 |
| 2.0 × 10⁶ MAGE-A4 TCR-T + REGN9903 (n = 8) | 1579.9 | 292.9 | 8 |
| 2.0 × 10⁶ MAGE-A4 TCR-T + REGN10597 (n = 9) | 130.7 | 88.8 | 9 |
| 1.0 × 10⁶ MAGE-A4 TCR-T (n = 8) | | | 0 |
| 1.0 × 10⁶ MAGE-A4 TCR-T + REGN9903 (n = 8) | | | 0 |
| 1.0 × 10⁶ MAGE-A4 TCR-T + REGN10597 (n = 9) | 1388.4 | 537.1 | 4 |

TABLE 13
Treatment effects on Day 39 of a combination therapy
of MAGE-A4 TCR-T cells + REGN10597 anti-PD1-IL2Ra-IL2

| | | Tumor size | Number of |
|---|---|---|---|
| | Average | standard | mice still |
| | tumor size | error of the | alive on |
| Treatment | on Day 39 | mean (SEM) | Day 39 |
| None (n = 5) | | | 0 |
| 4.0 × 10⁶ Control TCR-T (n = 5) | | | 0 |
| 4.0 × 10⁶ Control TCR-T + REGN9903 (n = 5) | | | 0 |
| 4.0 × 10⁶ Control TCR-T + REGN10597 (n = 5) | | | 0 |
| 4.0 × 10⁶ MAGE-A4 TCR-T (n = 8) | 768.1 | 192.2 | 8 |
| 4.0 × 10⁶ MAGE-A4 TCR-T + REGN9903 (n = 7) | 476.4 | 218.5 | 6 |
| 4.0 × 10⁶ MAGE-A4 TCR-T + REGN10597 (n = 9) | 0.0 | 0.0 | 8 |
| 2.0 × 10⁶ MAGE-A4 TCR-T (n = 8) | 2151.9 | 314.1 | 8 |
| 2.0 × 10⁶ MAGE-A4 TCR-T + REGN9903 (n = 8) | | | 0 |
| 2.0 × 10⁶ MAGE-A4 TCR-T + REGN10597 (n = 9) | 229.8 | 141.3 | 9 |
| 1.0 × 10⁶ MAGE-A4 TCR-T (n = 8) | | | 0 |
| 1.0 × 10⁶ MAGE-A4 TCR-T + REGN9903 (n = 8) | | | 0 |
| 1.0 × 10⁶ MAGE-A4 TCR-T + REGN10597 (n = 9) | 885.8 | 111.5 | 2 |

TABLE 14
Treatment effects on Day 42 of a combination therapy
of MAGE-A4 TCR-T cells + REGN10597 anti-PD1-IL2Ra-IL2

| | | Tumor size | Number of |
|---|---|---|---|
| | Average | standard | mice still |
| | tumor size | error of the | alive on |
| Treatment | on Day 42 | mean (SEM) | Day 42 |
| None (n = 5) | | | 0 |
| 4.0 × 10⁶ Control TCR-T (n = 5) | | | 0 |
| 4.0 × 10⁶ Control TCR-T + REGN9903 (n = 5) | | | 0 |
| 4.0 × 10⁶ Control TCR-T + REGN10597 (n = 5) | | | 0 |
| 4.0 × 10⁶ MAGE-A4 TCR-T (n = 8) | 924.4 | 241.7 | 8 |
| 4.0 × 10⁶ MAGE-A4 TCR-T + REGN9903 (n = 7) | 638.1 | 288.6 | 6 |
| 4.0 × 10⁶ MAGE-A4 TCR-T + REGN10597 (n = 9) | 0.0 | 0.0 | 8 |
| 2.0 × 10⁶ MAGE-A4 TCR-T (n = 8) | | | 0 |
| 2.0 × 10⁶ MAGE-A4 TCR-T + REGN9903 (n = 8) | | | 0 |
| 2.0 × 10⁶ MAGE-A4 TCR-T + REGN10597 (n = 9) | 345.5 | 193.3 | 9 |
| 1.0 × 10⁶ MAGE-A4 TCR-T (n = 8) | | | 0 |
| 1.0 × 10⁶ MAGE-A4 TCR-T + REGN9903 (n = 8) | | | 0 |
| 1.0 × 10⁶ MAGE-A4 TCR-T + REGN10597 (n = 9) | 1379.4 | 77.5 | 2 |

TABLE 15
Treatment effects on Day 46 of a combination therapy
of MAGE-A4 TCR-T cells + REGN10597 anti-PD1-IL2Ra-IL2

| | | Tumor size | Number of |
|---|---|---|---|
| | Average | standard | mice still |
| | tumor size | error of the | alive on |
| Treatment | on Day 46 | mean (SEM) | Day 46 |
| None (n = 5) | | | 0 |
| 4.0 × 10⁶ Control TCR-T (n = 5) | | | 0 |
| 4.0 × 10⁶ Control TCR-T + REGN9903 (n = 5) | | | 0 |
| 4.0 × 10⁶ Control TCR-T + REGN10597 (n = 5) | | | 0 |
| 4.0 × 10⁶ MAGE-A4 TCR-T (n = 8) | 1258.7 | 411.0 | 7 |
| 4.0 × 10⁶ MAGE-A4 TCR-T + REGN9903 (n = 7) | 851.3 | 393.5 | 6 |
| 4.0 × 10⁶ MAGE-A4 TCR-T + REGN10597 (n = 9) | 0.0 | 0.0 | 8 |
| 2.0 × 10⁶ MAGE-A4 TCR-T (n = 8) | | | 0 |
| 2.0 × 10⁶ MAGE-A4 TCR-T + REGN9903 (n = 8) | | | 0 |
| 2.0 × 10⁶ MAGE-A4 TCR-T + REGN10597 (n = 9) | 527.7 | 301.8 | 8 |
| 1.0 × 10⁶ MAGE-A4 TCR-T (n = 8) | | | 0 |
| 1.0 × 10⁶ MAGE-A4 TCR-T + REGN9903 (n = 8) | | | 0 |
| 1.0 × 10⁶ MAGE-A4 TCR-T + REGN10597 (n = 9) | 2191.9 | 299.2 | 2 |

TABLE 16
Treatment effects on Day 49 of a combination therapy
of MAGE-A4 TCR-T cells + REGN10597 anti-PD1-IL2Ra-IL2

| | | Tumor size | Number of |
|---|---|---|---|
| | Average | standard | mice still |
| | tumor size | error of the | alive on |
| Treatment | on Day 49 | mean (SEM) | Day 49 |
| None (n = 5) | | | 0 |
| 4.0 × 10⁶ Control TCR-T (n = 5) | | | 0 |
| 4.0 × 10⁶ Control TCR-T + REGN9903 (n = 5) | | | 0 |
| 4.0 × 10⁶ Control TCR-T + REGN10597 (n = 5) | | | 0 |
| 4.0 × 10⁶ MAGE-A4 TCR-T (n = 8) | 1571.7 | 527.3 | 7 |
| 4.0 × 10⁶ MAGE-A4 TCR-T + REGN9903 (n = 7) | 1128.5 | 532.5 | 6 |
| 4.0 × 10⁶ MAGE-A4 TCR-T + REGN10597 (n = 9) | 0.0 | 0.0 | 8 |
| 2.0 × 10⁶ MAGE-A4 TCR-T (n = 8) | | | 0 |
| 2.0 × 10⁶ MAGE-A4 TCR-T + REGN9903 (n = 8) | | | 0 |
| 2.0 × 10⁶ MAGE-A4 TCR-T + REGN10597 (n = 9) | 357.0 | 194.7 | 8 |
| 1.0 × 10⁶ MAGE-A4 TCR-T (n = 8) | | | 0 |
| 1.0 × 10⁶ MAGE-A4 TCR-T + REGN9903 (n = 8) | | | 0 |
| 1.0 × 10⁶ MAGE-A4 TCR-T + REGN10597 (n = 9) | | | 0 |

TABLE 17
Treatment effects of a combination therapy of MAGE-A4 TCR-T cells + REGN10597
anti-PD1-IL2Ra-IL2 as measured by tumor size analyzed by two-way ANOVA

| Tumor size analyzed | P value on each day | | | | | |
|---|---|---|---|---|---|---|
| by two-way ANOVA | Day 20 | Day 26 | Day 31 | Day 34 | Day 39 | Day 42 |
| 4 × 10⁶ TCR T vs | 0.5598 | 0.5837 | 0.606 | 0.6263 | 0.5904 | 0.734 |
| 4 × 10⁶ TCR T + REGN9903 | | | | | | |
| 4 × 10⁶ TCR T vs | 0.0272 | 0.0123 | 0.0084 | 0.0159 | 0.0126 | 0.0157 |
| 4 × 10⁶ TCR T + REGN10597 | | | | | | |
| 4 × 10⁶ TCR T + REGN9903 vs | 0.1933 | 0.0863 | 0.1238 | 0.1594 | 0.168 | 0.1621 |
| 4 × 10⁶ TCR T + REGN10597 | | | | | | |
| 2 × 10⁶ TCR T vs | 0.1197 | 0.033 | 0.0432 | 0.0217 | | |
| 2 × 10⁶ TCR T + REGN9903 | | | | | | |
| 2 × 10⁶ TCR T vs | 0.0191 | 0.0003 | <0.0001 | 0.0082 | | |
| 2 × 10⁶ TCR T + REGN10597 | | | | | | |
| 2 × 10⁶ TCR T + REGN9903 vs | 0.0724 | 0.0012 | 0.0026 | 0.0253 | | |
| 2 × 10⁶ TCR T + REGN10597 | | | | | | |
| 1 × 10⁶ TCR T vs | 0.2553 | | | | | |
| 1 × 10⁶ TCR T + REGN9903 | | | | | | |
| 1 × 10⁶ TCR T vs | 0.0294 | | | | | |
| 1 × 10⁶ TCR T + REGN10597 | | | | | | |
| 1 × 10⁶ TCR T + REGN9903 vs | 0.4775 | | | | | |
| 1 × 10⁶ TCR T + REGN10597 | | | | | | |

### Example 3: Synergistic Anti-Tumor Efficacy of the Combination Therapy of Anti-huCD20 CAR-T Cells+Anti-PD1-IL2Ra-IL2 (REGN10597)

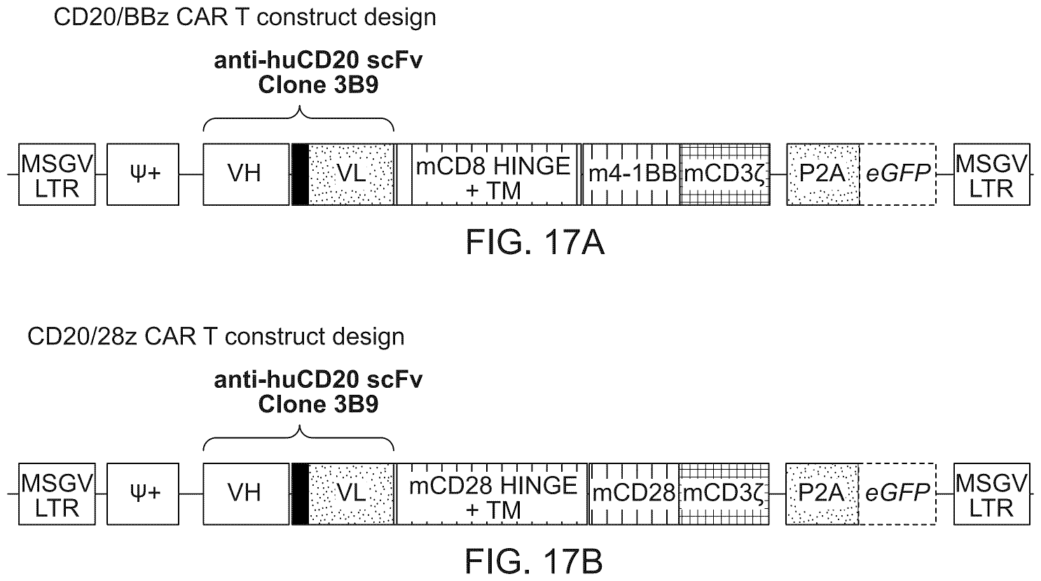

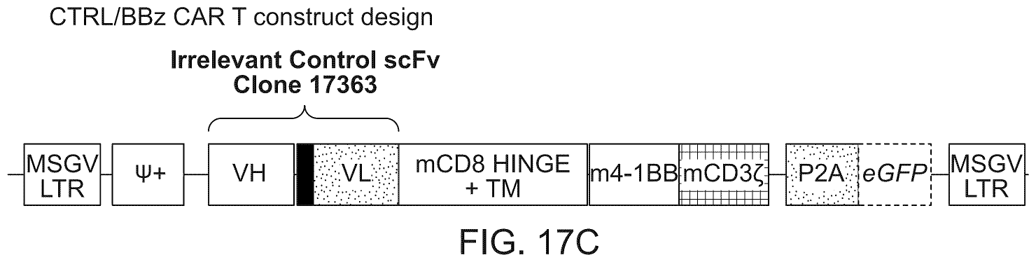

CD3+ T cells were isolated from the spleens of C57BL/6 mice expressing human PD-1 in place of murine PD-1 (PD-1-humanized mice), and stimulated with CD3/CD28 microbeads plus recombinant human IL-2 before transduction with retroviruses expressing various CAR constructs. The cells were then cultured with IL7 and IL15 and expanded further before cryopreservation. T cells were engineered to express one of three CARs: (1) anti-huCD20 CAR-T with CD3z and 4-1BB signaling domains (CD20/BBz CAR-T); (2) anti-huCD20 CAR-T with CD3z and CD28 signaling domains (CD20/28z CAR-T); and (3) Control CAR-T with CD3z and 4-1BB signaling domains (CTRL/BBz CAR-T). Schematics of these CAR constructs are shown in FIGS. 17A-17C.

Table 18 sets forth the amino acid sequences of CAR constructs CD2/BBz CAR-T and CTRL/BBz CAR-T.

TABLE 18
Amino acid sequences of CAR constructs
CD20/BBz CAR-T and CTRL/BBz CAR-T

| SEQ | | |
|---|---|---|
| ID | | |
| NO | AMINO ACID SEQUENCE | INFORMATION |
| 43 | MGVPTQLLGLLLLWITDAICEIVMT | anti-huCD20 |
| | QSPATLSVSPGERATLSCRASQSVS | CAR-T with |
| | SNLAWYQQKPGQAPRLLIYGTSTRA | CD3z and 4- |
| | TGIPARFSGSGSGTEFTLTISSLQS | 1BB signaling |
| | EDFAVYYCQQYNNWPLTFGGGTKVE | domains |
| | IKGGGGSGGGGSGGGGSEVQLVESG | (CD20/BBz |
| | GGLVQPGRSLRLSCVASGFTFNDYA | CAR-T) |
| | MHWVRQAPGKGLEWVSVISWNSDSI | |
| | GYADSVKGRFTISRDNAKNSLYLQM | |
| | HSLRAEDTALYYCAKDNHYGSGSYY | |
| | YYQYGMDVWGQGTTVTVSSGGGGST | |
| | TTKPVLRTPSPVHPTGTSQPQRPED | |
| | CRPRGSVKGTGLDFACDIYIWAPLA | |
| | GICVALLLSLIITLICYHRSRKWIR | |
| | KKFPHIFKQPFKKTTGAAQEEDACS | |
| | CRCPQEEEGGGGGYELRAKFSRSAE | |
| | TAANLQDPNQLYNELNLGRREEYDV | |
| | LEKKRARDPEMGGKQQRRRNPQEGV | |
| | YNALQKDKMAEAYSEIGTKGERRRG | |
| | KGHDGLYQGLSTATKDTYDALHMQT | |
| | LAPRGSGATNFSLLKQAGDVEENPG | |
| | PMVSKGEELFTGVVPILVELDGDVN | |
| | GHKFSVSGEGEGDATYGKLTLKFIC | |
| | TTGKLPVPWPTLVTTLTYGVQCFSR | |
| | YPDHMKQHDFFKSAMPEGYVQERTI | |
| | FFKDDGNYKTRAEVKFEGDTLVNRI | |
| | ELKGIDFKEDGNILGHKLEYNYNSH | |
| | NVYIMADKQKNGIKVNFKIRHNIED | |
| | GSVQLADHYQQNTPIGDGPVLLPDN | |
| | HYLSTQSALSKDPNEKRDHMVLLEF | |
| | VTAAGITLGMDELYK | |
| 44 | EIVMTQSPATLSVSPGERATLSCRA | Anti-CD20 VK |
| | SQSVSSNLAWYQQKPGQAPRLLIYG | |
| | TSTRATGIPARFSGSGSGTEFTLTI | |
| | SSLQSEDFAVYYCQQYNNWPLTFGG | |
| | GTKVEIK | |
| 45 | EVQLVESGGGLVQPGRSLRLSCVAS | Anti-CD20 VH |
| | GFTFNDYAMHWVRQAPGKGLEWVSV | |
| | ISWNSDSIGYADSVKGRFTISRDNA | |
| | KNSLYLQMHSLRAEDTALYYCAKDN | |
| | HYGSGSYYYYQYGMDVWGQGTTVTV | |
| | SS | |
| 46 | MGVPTQLLGLLLLWITDAICDIQMT | CTRL mAb |
| | QSPSSLSASVGDRVTITCRASQSIS | CAR-T with |
| | SYLNWYQQKPGKAPKLLIYAVSILQ | CD3z and 4- |
| | SGVPSRFSGSGSGTDFTLTINSLQP | 1BB signaling |
| | EDFATYSCQQTYSTPPITFGQGTRL | domains |
| | EIKGGGGSGGGGSGGGGSEVQLLES | (CTRL/BBz |
| | GGGLVQPGGSLRLSCAASGFTFSSY | CAR-T) |
| | AMTWVRQAPGMGLEWVSVISGSGSE | |
| | TYYADSVKGRFTISRDNSKNTLYLQ | |
| | MNSLRAEDTAVYYCVKDSSYRSSSR | |
| | AYYYYGMDVWGLGTTVTVSSGGGGS | |
| | TTTKPVLRTPSPVHPTGTSQPQRPE | |
| | DCRPRGSVKGTGLDFACDIYIWAPL | |
| | AGICVALLLSLIITLICYHRSRKWI | |
| | RKKFPHIFKQPFKKTTGAAQEEDAC | |
| | SCRCPQEEEGGGGGYELRAKFSRSA | |
| | ETAANLQDPNQLYNELNLGRREEYD | |
| | VLEKKRARDPEMGGKQQRRRNPQEG | |
| | VYNALQKDKMAEAYSEIGTKGERRR | |
| | GKGHDGLYQGLSTATKDTYDALHMQ | |
| | TLAPRGSGATNFSLLKQAGDVEENP | |
| | GPMVSKGEELFTGVVPILVELDGDV | |
| | NGHKFSVSGEGEGDATYGKLTLKFI | |
| | CTTGKLPVPWPTLVTTLTYGVQCFS | |
| | RYPDHMKQHDFFKSAMPEGYVQERT | |
| | IFFKDDGNYKTRAEVKFEGDTLVNR | |
| | IELKGIDFKEDGNILGHKLEYNYNS | |
| | HNVYIMADKQKNGIKVNFKIRHNIE | |
| | DGSVQLADHYQQNTPIGDGPVLLPD | |
| | NHYLSTQSALSKDPNEKRDHMVLLE | |
| | FVTAAGITLGMDELYK | |
| 47 | DIQMTQSPSSLSASVGDRVTITCRA | CTRL mAb VK |
| | SQSISSYLNWYQQKPGKAPKLLIYA | |
| | VSILQSGVPSRFSGSGSGTDFTLTI | |
| | NSLQPEDFATYSCQQTYSTPPITFG | |
| | QGTRLEIK | |
| 48 | EVQLLESGGGLVQPGGSLRLSCAAS | CTRL mAb VH |
| | GFTFSSYAMTWVRQAPGMGLEWVSV | |
| | ISGSGSETYYADSVKGRFTISRDNS | |
| | KNTLYLQMNSLRAEDTAVYYCVKDS | |
| | SYRSSSRAYYYYGMDVWGLGTTVTV | |
| | SS | |
| 49 | MGVPTQLLGLLLLWITDAIC | Signal |
| | | Sequence |
| 50 | GGGGSGGGGSGGGGS | (G4S)3 |
| 51 | GGGGS | G4S |
| 52 | TTTKPVLRTPSPVHPTGTSQPQRPE | Mouse CD8 |
| | DCRPRGSVKGTGLDFACDIYIWAPL | hinge/transmem |
| | AGICVALLLSLIITLICYHRSR | brane |
| 53 | KWIRKKFPHIFKQPFKKTTGAAQEE | Mouse 4-1BB |
| | DACSCRCPQEEEGGGGGYEL | signaling |
| | | domain |
| 54 | RAKFSRSAETAANLQDPNQLYNELN | Mouse CD3z |
| | LGRREEYDVLEKKRARDPEMGGKQQ | signaling |
| | RRRNPQEGVYNALQKDKMAEAYSEI | domain |
| | GTKGERRRGKGHDGLYQGLSTATKD | |
| | TYDALHMQTLAPR | |
| 55 | GSGATNFSLLKQAGDVEENPGP | GSG and P2A |
| | | site |
| 56 | MVSKGEELFTGVVPILVELDGDVNG | GFP |
| | HKFSVSGEGEGDATYGKLTLKFICT | |
| | TGKLPVPWPTLVTTLTYGVQCFSRY | |
| | PDHMKQHDFFKSAMPEGYVQERTIF | |
| | FKDDGNYKTRAEVKFEGDTLVNRIE | |
| | LKGIDFKEDGNILGHKLEYNYNSHN | |
| | VYIMADKQKNGIKVNFKIRHNIEDG | |
| | SVQLADHYQQNTPIGDGPVLLPDNH | |
| | YLSTQSALSKDPNEKRDHMVLLEFV | |
| | TAAGITLGMDELYK | |

To determine the synergistic anti-tumor efficacy of CD20 CAR-T cells+anti-PD1-IL2Ra-IL2 (REGN10597), a syngeneic tumor study was performed. On Day −3, PD-1-humanized C57BL/6 mice were lymphodepleted with 250 mg/kg cyclophosphamide, and subsequently injected subcutaneously on Day 0 with 1×10⁶ MC38 murine colon carcinoma cells expressing human CD20 (MC38/hCD20 cells). On Day 4 after tumor implantation, mice were intravenously injected with freshly-thawed CAR-T cells. The mice received either 0.5×10⁶ CD20/BBz CAR-T cells, CD20/28z CAR-T cells, or CTRL/BBz CAR-T cells. Mice were then intraperitoneally treated with either anti-PD1-IL2Ra-IL2 (REGN10597) or a non-targeted CTRL-IL2Ra-IL2 (REGN9903) at either 0.2 or 0.5 mg/kg on days 7, 11, 14, and 18. Tumor volume was measured twice weekly using calipers and calculated by the formula: volume=(length×width²)/2. Mice were euthanized when tumor diameter exceeded 20 mm, in accordance with IACUC protocols.

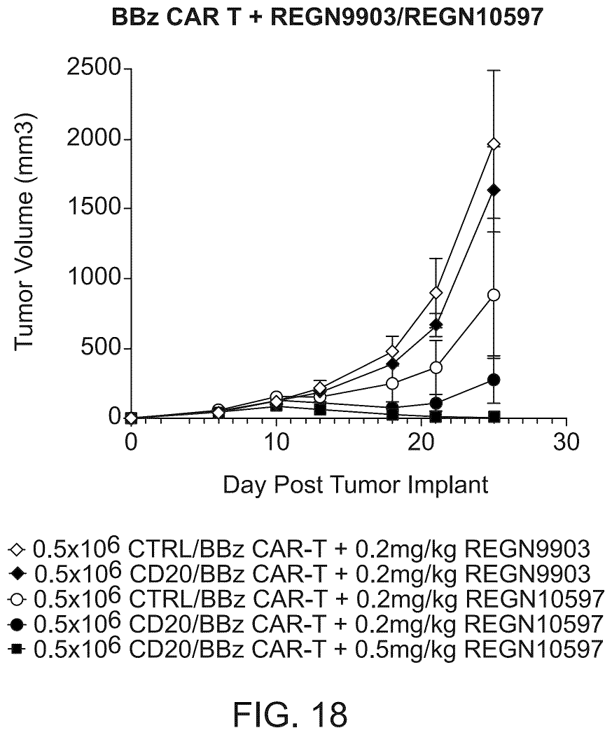

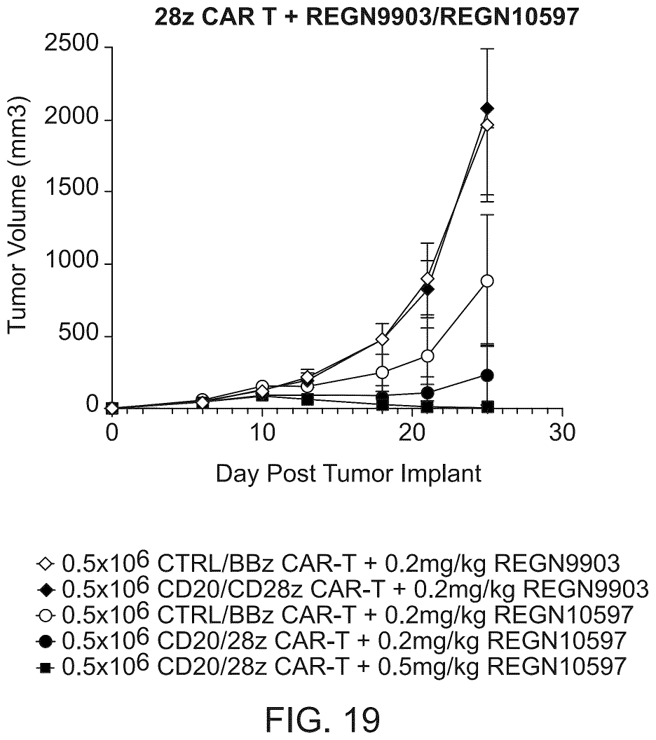

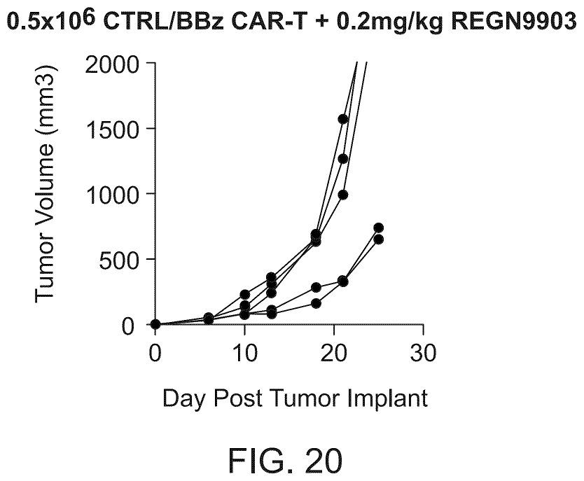

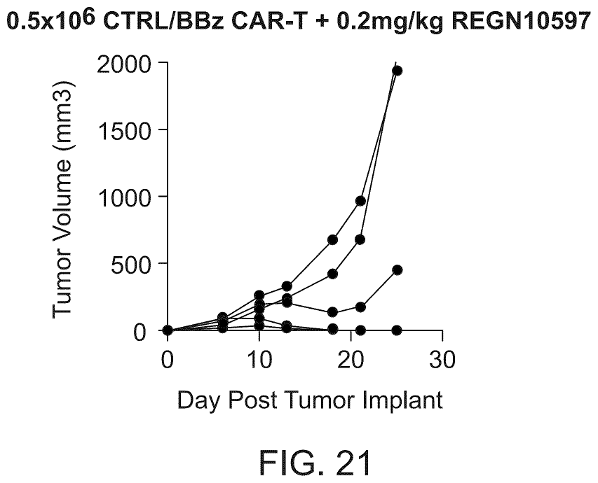

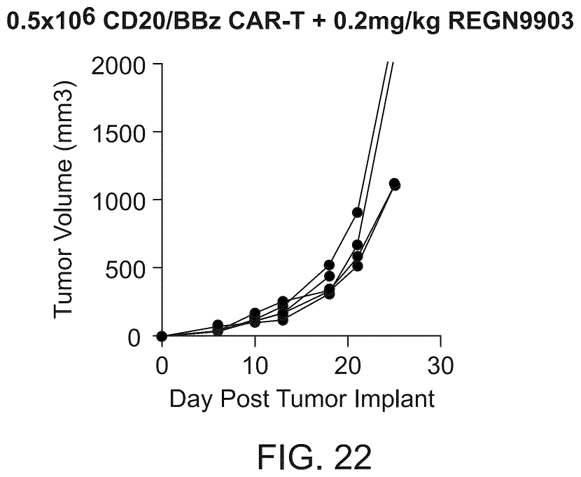

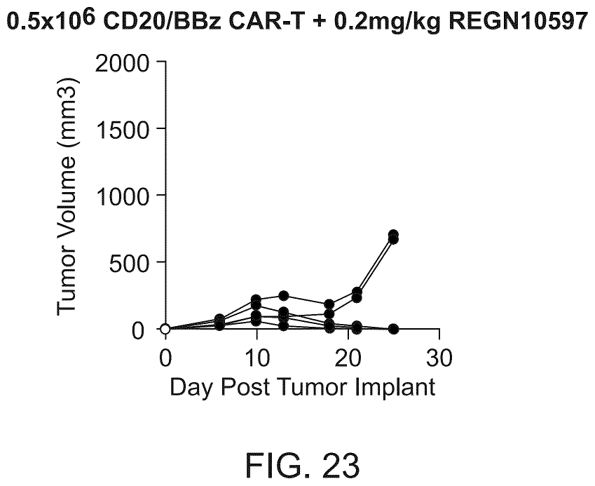

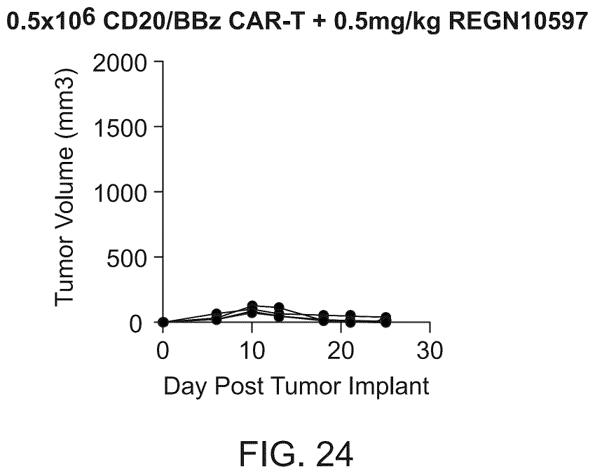

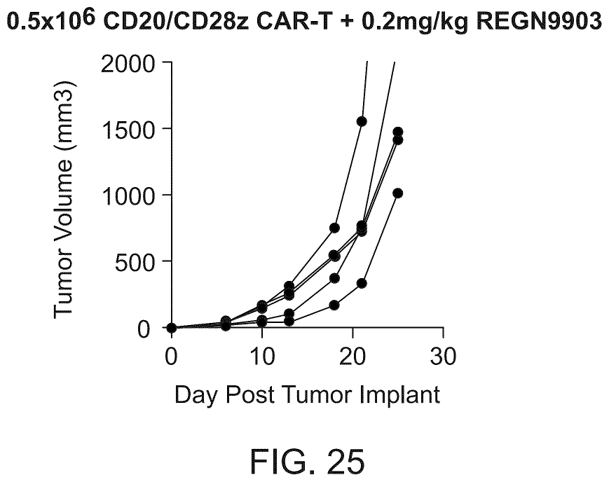

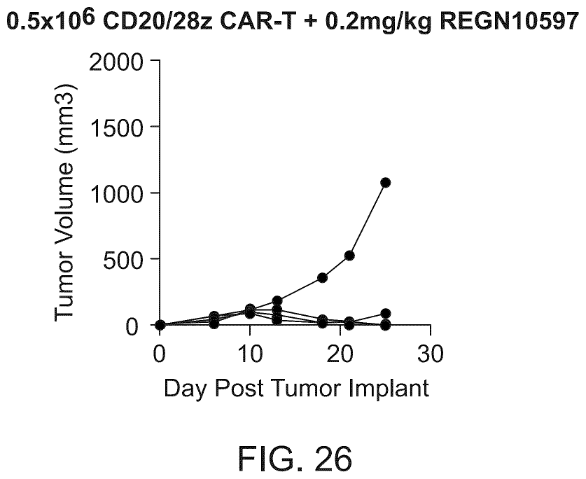

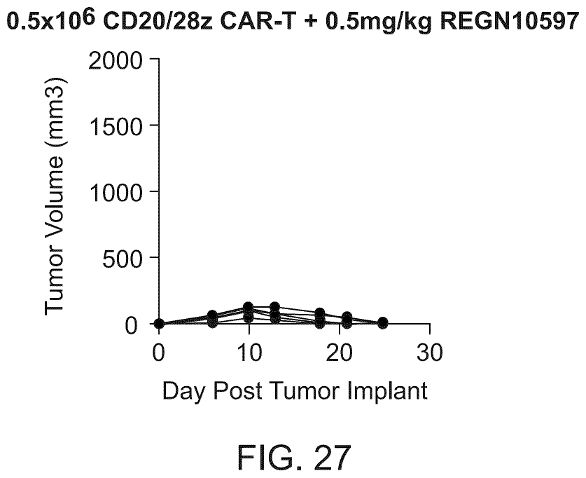

As shown in FIGS. 18-27 and Tables 19-24, MC38/hCD20 tumors grew progressively in mice receiving CTRL/BBz CAR-T plus CTRL-IL2Ra-IL2 (REGN9903; 0.2 mg/kg), CD20/BBz CAR-T plus CTRL-IL2Ra-IL2 (0.2 mg/kg), or CD20/28z CAR-T plus CTRL-IL2Ra-IL2 (0.2 mg/kg) (FIGS. 18-19). Tumor growth was only modestly reduced in mice receiving CTRL/BBz CAR-T plus PD1-IL2Ra-IL2 (REGN10597; 0.2 mg/kg) (FIGS. 18-19). However, tumor growth in mice receiving CD20/BBz CAR-T plus anti-PD1-IL2Ra-IL2 (0.2 mg/kg and 0.5 mg/kg) was significantly suppressed compared to mice receiving CD20/BBz CAR-T plus CTRL-IL2Ra-IL2 (0.2 mg/kg; p<0.0001 and p<0.001, respectively at day 25, by 2-way ANOVA analysis) (FIGS. 18-27). Tumor growth in mice receiving CD20/28z CAR-T plus anti-PD1-IL2Ra-IL2 (0.2 mg/kg and 0.5 mg/kg) was also significantly suppressed compared to mice receiving CD20/28z CAR-T plus CTRL-IL2Ra-IL2 (0.2 mg/kg; p<0.0001 and p<0.0001, respectively at day 25, by 2-way ANOVA analysis) (Table 24).

These data demonstrate that combining CAR-T cell therapy with anti-PD1-IL2Ra-IL2 (REGN10597) induces potent and durable tumor control compared to CAR-T cells alone. Accordingly, this representative example further supports the expectation that administration of ACT in combination with a targeted immunocytokine to a subject with cancer will lead to increased efficacy and duration of anti-tumor response, as compared to a subject treated with the ACT as monotherapy.

TABLE 19
Treatment effects on Day 6 of a combination therapy of
anti-huCD20 CAR-T cells + anti-PD1-IL2Ra-IL2 (REGN10597)

| | | Tumor size | Number of |
|---|---|---|---|
| | Average | standard | mice still |
| | tumor size | error of the | alive on |
| Treatment | on Day 6 | mean (SEM) | Day 6 |
| 0.5 × 10⁶ CTRL/BBz CAR-T + 0.2 mg/kg REGN9903 | 38.0 | 2.9 | 5 |
| 0.5 × 10⁶ CD20/BBz CAR-T + 0.2 mg/kg REGN9903 | 53.3 | 7.8 | 5 |
| 0.5 × 10⁶ CD20/CD28z CAR-T + 0.2 mg/kg REGN9903 | 36.6 | 5.2 | 5 |
| 0.5 × 10⁶ CTRL/BBz CAR-T + 0.2 mg/kg REGN10597 | 58.7 | 13.4 | 5 |
| 0.5 × 10⁶ CD20/BBz CAR-T + 0.2 mg/kg REGN10597 | 42.7 | 9.5 | 5 |
| 0.5 × 10⁶ CD20/28z CAR-T + 0.2 mg/kg REGN10597 | 43.3 | 7.9 | 5 |
| 0.5 × 10⁶ CD20/BBz CAR-T + 0.5 mg/kg REGN10597 | 39.1 | 7.1 | 5 |
| 0.5 × 10⁶ CD20/28z CAR-T + 0.5 mg/kg REGN10597 | 43.0 | 9.6 | 5 |

TABLE 20
Treatment effects on Day 10 of a combination therapy of
anti-huCD20 CAR-T cells + anti-PD1-IL2Ra-IL2 (REGN10597)

| | | Tumor size | Number of |
|---|---|---|---|
| | Average | standard | mice still |
| | tumor size | error of the | alive on |
| Treatment | on Day 10 | mean (SEM) | Day 10 |
| 0.5 × 10⁶ CTRL/BBz CAR-T + 0.2 mg/kg REGN9903 | 119.5 | 29.0 | 5 |
| 0.5 × 10⁶ CD20/BBz CAR-T + 0.2 mg/kg REGN9903 | 125.7 | 10.8 | 5 |
| 0.5 × 10⁶ CD20/CD28z CAR-T + 0.2 mg/kg REGN9903 | 116.3 | 26.9 | 5 |
| 0.5 × 10⁶ CTRL/BBz CAR-T + 0.2 mg/kg REGN10597 | 148.3 | 39.2 | 5 |
| 0.5 × 10⁶ CD20/BBz CAR-T + 0.2 mg/kg REGN10597 | 129.0 | 29.5 | 5 |
| 0.5 × 10⁶ CD20/28z CAR-T + 0.2 mg/kg REGN10597 | 100.9 | 5.8 | 5 |
| 0.5 × 10⁶ CD20/BBz CAR-T + 0.5 mg/kg REGN10597 | 93.8 | 9.3 | 5 |
| 0.5 × 10⁶ CD20/28z CAR-T + 0.5 mg/kg REGN10597 | 96.9 | 13.3 | 5 |

TABLE 21
Treatment effects on Day 13 of a combination therapy of
anti-huCD20 CAR-T cells + anti-PD1-IL2Ra-IL2 (REGN10597)

| | | Tumor size | Number of |
|---|---|---|---|
| | Average | standard | mice still |
| | tumor size | error of the | alive on |
| Treatment | on Day 13 | mean (SEM) | Day 13 |
| 0.5 × 10⁶ CTRL/BBz CAR-T + 0.2 mg/kg REGN9903 | 217.8 | 54.7 | 5 |
| 0.5 × 10⁶ CD20/BBz CAR-T + 0.2 mg/kg REGN9903 | 185.3 | 23.6 | 5 |
| 0.5 × 10⁶ CD20/CD28z CAR-T + 0.2 mg/kg REGN9903 | 196.0 | 50.5 | 5 |
| 0.5 × 10⁶ CTRL/BBz CAR-T + 0.2 mg/kg REGN10597 | 163.8 | 60.5 | 5 |
| 0.5 × 10⁶ CD20/BBz CAR-T + 0.2 mg/kg REGN10597 | 113.1 | 37.5 | 5 |
| 0.5 × 10⁶ CD20/28z CAR-T + 0.2 mg/kg REGN10597 | 94.0 | 25.0 | 5 |
| 0.5 × 10⁶ CD20/BBz CAR-T + 0.5 mg/kg REGN10597 | 64.6 | 13.0 | 5 |
| 0.5 × 10⁶ CD20/28z CAR-T + 0.5 mg/kg REGN10597 | 73.3 | 16.2 | 5 |

TABLE 22
Treatment effects on Day 18 of a combination therapy of
anti-huCD20 CAR-T cells + anti-PD1-IL2Ra-IL2 (REGN10597)

| | | Tumor size | Number of |
|---|---|---|---|
| | Average | standard | mice still |
| | tumor size | error of the | alive on |
| Treatment | on Day 18 | mean (SEM) | Day 18 |
| 0.5 × 10⁶ CTRL/BBz CAR-T + 0.2 mg/kg REGN9903 | 481.7 | 109.3 | 5 |
| 0.5 × 10⁶ CD20/BBz CAR-T + 0.2 mg/kg REGN9903 | 390.2 | 38.9 | 5 |
| 0.5 × 10⁶ CD20/CD28z CAR-T + 0.2 mg/kg REGN9903 | 475.6 | 96.6 | 5 |
| 0.5 × 10⁶ CTRL/BBz CAR-T + 0.2 mg/kg REGN10597 | 249.5 | 130.5 | 5 |
| 0.5 × 10⁶ CD20/BBz CAR-T + 0.2 mg/kg REGN10597 | 73.1 | 33.2 | 5 |
| 0.5 × 10⁶ CD20/28z CAR-T + 0.2 mg/kg REGN10597 | 90.0 | 66.6 | 5 |
| 0.5 × 10⁶ CD20/BBz CAR-T + 0.5 mg/kg REGN10597 | 28.5 | 7.4 | 5 |
| 0.5 × 10⁶ CD20/28z CAR-T + 0.5 mg/kg REGN10597 | 37.3 | 16.1 | 5 |

TABLE 23
Treatment effects on Day 21 of a combination therapy of
anti-huCD20 CAR-T cells + anti-PD1-IL2Ra-IL2 (REGN10597)

| | | Tumor size | Number of |
|---|---|---|---|
| | Average | standard | mice still |
| | tumor size | error of the | alive on |
| Treatment | on Day 21 | mean (SEM) | Day 21 |
| 0.5 × 10⁶ CTRL/BBz CAR-T + 0.2 mg/kg REGN9903 | 896.9 | 249.5 | 5 |
| 0.5 × 10⁶ CD20/BBz CAR-T + 0.2 mg/kg REGN9903 | 668.8 | 84.9 | 4 |
| 0.5 × 10⁶ CD20/CD28z CAR-T + 0.2 mg/kg REGN9903 | 825.7 | 197.7 | 5 |
| 0.5 × 10⁶ CTRL/BBz CAR-T + 0.2 mg/kg REGN10597 | 363.9 | 195.3 | 5 |
| 0.5 × 10⁶ CD20/BBz CAR-T + 0.2 mg/kg REGN10597 | 109.7 | 60.2 | 5 |
| 0.5 × 10⁶ CD20/28z CAR-T + 0.2 mg/kg REGN10597 | 116.4 | 102.3 | 5 |
| 0.5 × 10⁶ CD20/BBz CAR-T + 0.5 mg/kg REGN10597 | 13.5 | 10.1 | 5 |
| 0.5 × 10⁶ CD20/28z CAR-T + 0.5 mg/kg REGN10597 | 16.7 | 9.3 | 5 |

TABLE 24
Treatment effects on Day 25 of a combination therapy of
anti-huCD20 CAR-T cells + anti-PD1-IL2Ra-IL2 (REGN10597)

| | | Tumor size | Number of |
|---|---|---|---|
| | Average | standard | mice still |
| | tumor size | error of the | alive on |
| Treatment | on Day 25 | mean (SEM) | Day 25 |
| 0.5 × 10⁶ CTRL/BBz CAR-T + 0.2 mg/kg REGN9903 | 1963.8 | 528.7 | 5 |
| 0.5 × 10⁶ CD20/BBz CAR-T + 0.2 mg/kg REGN9903 | 1638.6 | 308.3 | 4 |
| 0.5 × 10⁶ CD20/CD28z CAR-T + 0.2 mg/kg REGN9903 | 2074.7 | 592.8 | 5 |
| 0.5 × 10⁶ CTRL/BBz CAR-T + 0.2 mg/kg REGN10597 | 885.1 | 458.2 | 5 |
| 0.5 × 10⁶ CD20/BBz CAR-T + 0.2 mg/kg REGN10597 | 274.7 | 168.3 | 5 |
| 0.5 × 10⁶ CD20/28z CAR-T + 0.2 mg/kg REGN10597 | 231.3 | 211.5 | 5 |
| 0.5 × 10⁶ CD20/BBz CAR-T + 0.5 mg/kg REGN10597 | 10.5 | 7.0 | 5 |
| 0.5 × 10⁶ CD20/28z CAR-T + 0.5 mg/kg REGN10597 | 2.6 | 2.6 | 5 |

### Example 4: Synergistic Efficacy of Anti-huCD20 CAR T Cells in Combination with PD1-IL2Ra-IL2 to Drive Superior and More Durable Depletion of Target Cells

This example relates to an in vivo study performed to demonstrate the ability of a PD1-targeted IL-2 immunocytokine (PD1-IL2Ra-IL2) to drive superior and more durable depletion of target cells in combination with an anti-huCD20 CAR T cell therapy compared to CAR T cells alone in the context of lymphodepletion as well as without lymphodepletion.

Lymphodepletion via administration of chemotherapeutic agents is commonly used in the CAR T field to facilitate engraftment of transferred cells by creating physical space and by removing cellular sinks to make available excess growth/survival factors (such as cytokines). However, lymphodepletion is associated with side effects that may prevent less fit patients from qualifying for CAR T therapy. Thus, a therapy that allows efficient CAR T cell engraftment/activity without the need for lymphodepletion is desirable. Therefore, in this study, the ability of PD1-IL2Ra-IL2 to drive superior and more durable depletion of target cells in vivo in combination with CAR T cells was tested both in the context of lymphodepletion (via cyclophosphamide treatment) as well as without lymphodepletion.

The present study was performed in immunocompetent C57BL/6 mice humanized for CD20 expression, where B cell depletion mediated by CAR T cells can be measured. In this model, the depletion of endogenous B cells by CAR T represents a surrogate for the depletion of huCD20⁺ tumor cells. Because these animals express murine PD1, a surrogate PD1-IL2Ra-IL2 reagent was used (i.e., REGN9899, Table 25), which binds to murine PD-1. The mouse PD1 binding moiety is derived from rat anti-mPD-1 clone RMP1-14, and a corresponding non-targeting NT-IL2Ra-IL2 reagent was used (i.e., REGN9901, Table 26).

Table 25 sets forth a description of REGN9899.

TABLE 25
Description of REGN9899

| Anti-mPD1- | | |
|---|---|---|
| IL2Ra-IL2 | Anti-PD1 antigen binding domain | Anti-PD1 antigen binding domain |
| REGN9899 | Heavy Chain: | Light Chain: |
| | anti-PD1 RMP1-14 VH(rat).mIgG1.3xG4S | anti-PD1 RMP1-14 VK(rat).mKappa |
| | linker.hIL2Ra.5xG4S linker.hIL-2 | |

TABLE 26
Description of REGN9901

| NT-IL2Ra-IL2 | Non-targeted antigen binding domain | Non-targeted antigen binding domain |
|---|---|---|
| REGN9901 | Heavy Chain: | Light Chain: |
| | VBZ13H2(1)_VH(mouse).mIgG1.3xG4S | AC13162 - |
| | linker.hIL2Ra.5xG4S linker.hIL-2 | VBZ13H2(1)_VK(mouse).mKappa_v2 |

To generate murine anti-huCD20 CAR T cells, CD3⁺ T cells were isolated from the spleens of huCD3/huCD20 knock-in mice using an untouched mouse T-cell isolation kit (Invitrogen #11413D) before activation with CD3/CD28 Dynabeads (Invitrogen #11161D) and recombinant human IL-2 (20 U/ml; Peprotech #200-02). After 16 hours, the T cells were transduced via spin infection on plates coated with Retronectin (Takara #T100B) with retrovirus encoding an anti-huCD20 CAR containing murine CD3z and mouse 4-1BB intracellular signaling domains. CAR T cells that bind an irrelevant antigen were used as controls. The CAR T cells included a GFP reporter (via P2A cleavage site) so that CAR T cells could be identified in vivo. CAR T cells used in this study are: anti-huCD20 CAR T with CD3z and 4-1BB signaling domains (CD20/BBz CAR-T, FIG. 17A; Table 18), and Control CAR T with CD3z and 4-1BB signaling domains (CTRL/BBz CAR-T, FIG. 17C, Table 18).

CD20-humanized mice were either lymphodepleted with an intraperitoneal dose of cyclophosphamide (250 mg/kg) or left untreated on Day −7, before intravenous injection with 3×10⁶ CAR⁺ anti-huCD20 CAR T or control CAR T cells on Day 0. The mice received the first dose of either PD1-IL2Ra-IL2 (i.e., REGN9899) or a control, non-targeting NT-IL2Ra-IL2 (i.e., REGN9901) intraperitoneally on Day 1 (0.4 mg/kg for the lymphodepleted groups, or 1 mg/kg for non-lymphodepleted groups). The mice then continued to receive the same doses of REGN9899 or REGN9901 every 3-4 days throughout the course of the study. The mice were bled to assess the frequencies and absolute numbers of CD45⁺B220⁺ B cells and CD45⁺CD90.2⁺GFP⁺ CAR T cells on Days 7 and 21 using immunofluorescence staining with flow cytometry analysis.

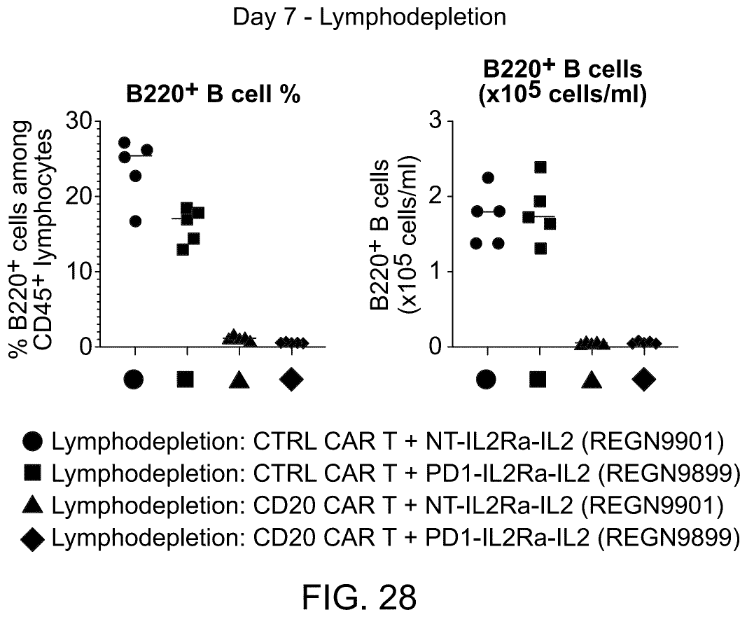

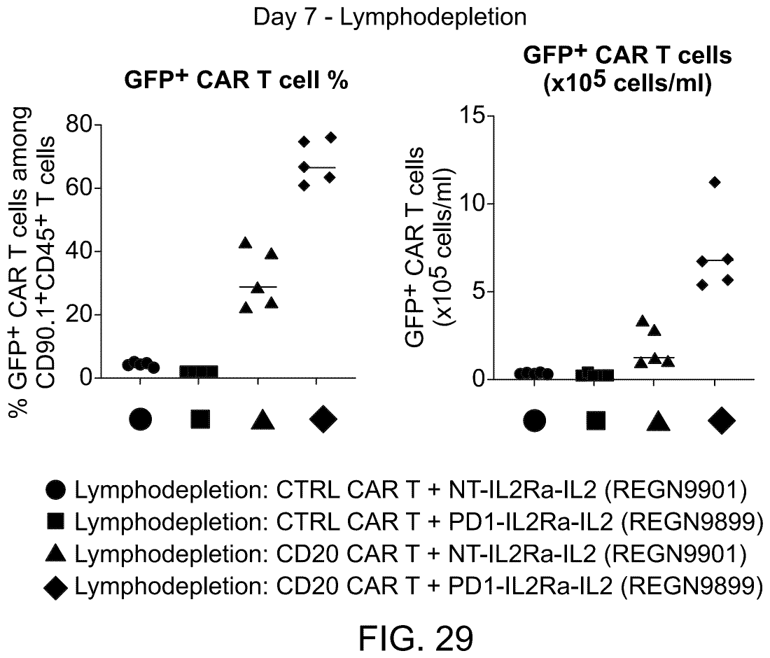

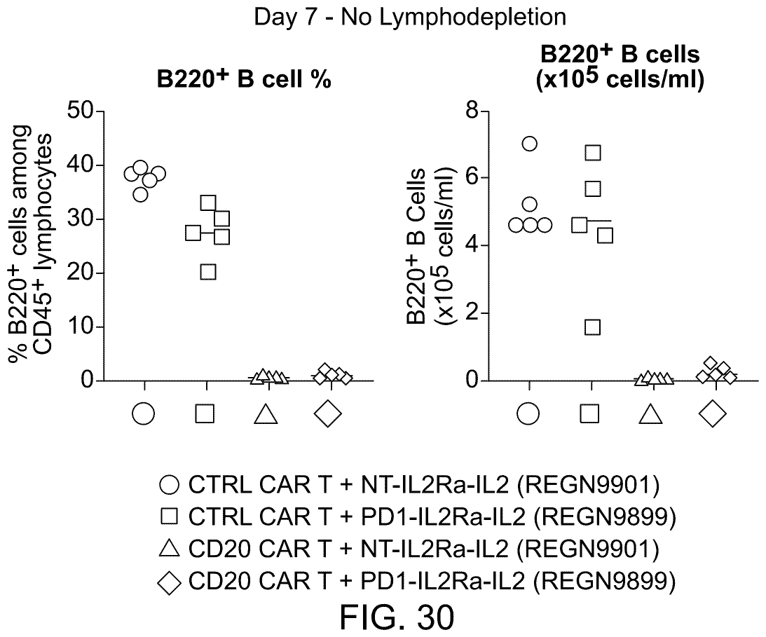

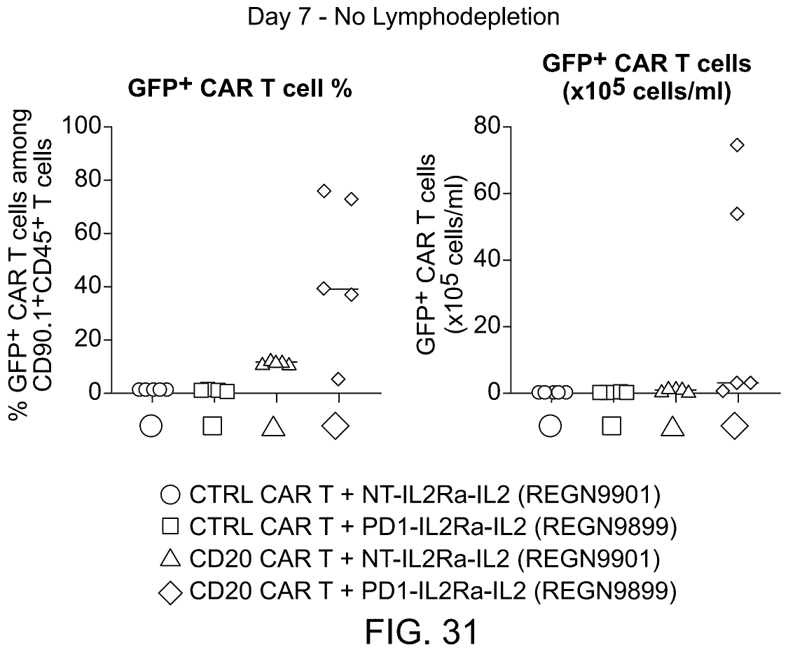

Results: On Day 7, treatment with anti-huCD20 CART cells efficiently depleted both the frequency and absolute number of peripheral blood B220⁺ B cells compared to CTRL CAR T, regardless of whether REGN9899 was administered (Tables 27 and 28; FIGS. 28-31). B cell depletion was also efficient regardless of whether the mice were lymphodepleted (Tables 27 and 28; FIGS. 28 and 30). In lymphodepleted mice, peripheral blood CAR T cell frequencies and absolute numbers were elevated compared to mice receiving CTRL CAR T cells (Table 27; FIG. 29), consistent antigen-specific recognition and activation/expansion of the CAR T. In these lymphodepleted mice, the frequency (p=0.0001) and absolute number (p=0.0019) of peripheral blood CAR T cells was significantly increased in mice receiving REGN9899 compared to mice receiving REGN9901, as assessed by a two-tailed, unpaired T-test, demonstrating that treatment with REGN9899 drives superior peripheral CAR T cell expansion/persistence. In non-lymphodepleted mice, less peripheral CAR T expansion was noted, but REGN9899 did drive increased frequencies of CAR T cells (p=0.0305) compared to REGN9901-treated mice, as determined by a two-tailed, unpaired T-test.

Day 7 summary: At this early timepoint, huCD20 CAR T-mediated B cell depletion in blood was efficient regardless of whether the mice were lymphodepleted and whether they received REGN9899. However, co-treatment with REGN9899 drove enhanced peripheral CAR T cell expansion compared to REGN9901-treated mice, especially in the context of lymphodepletion.

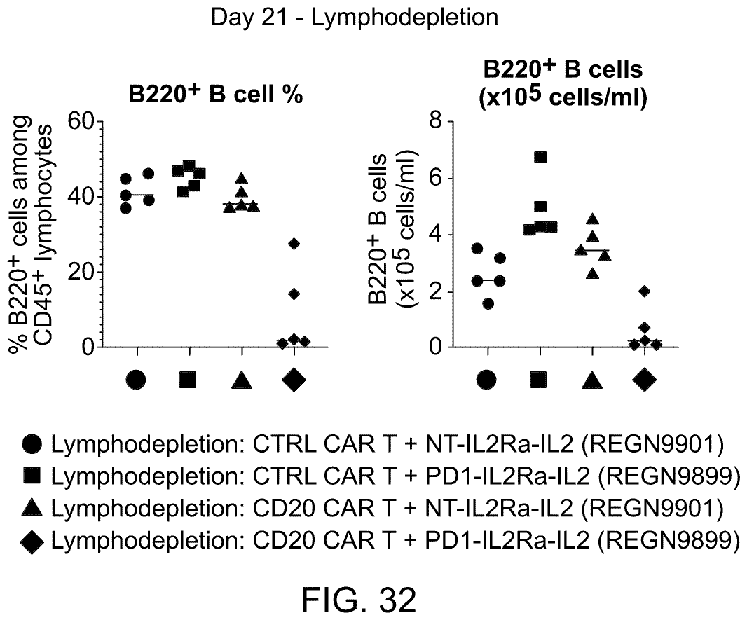

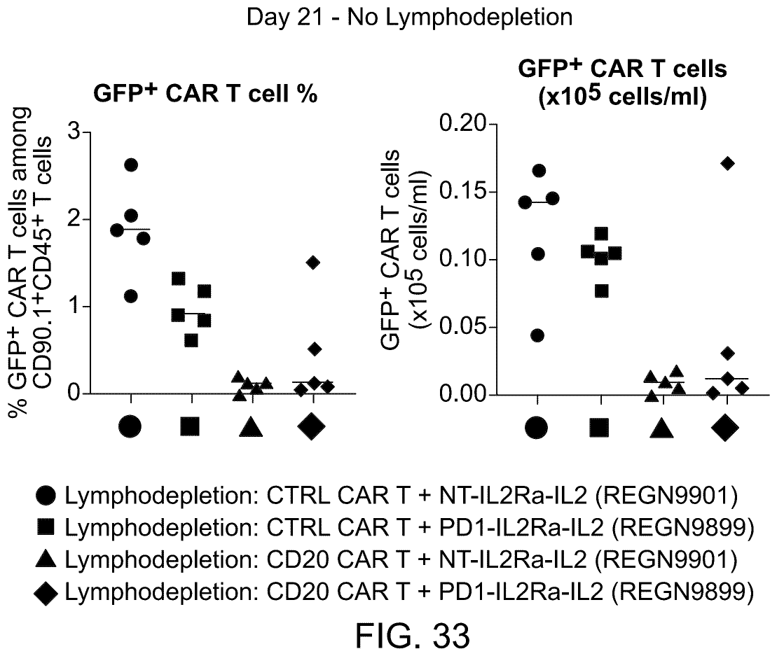

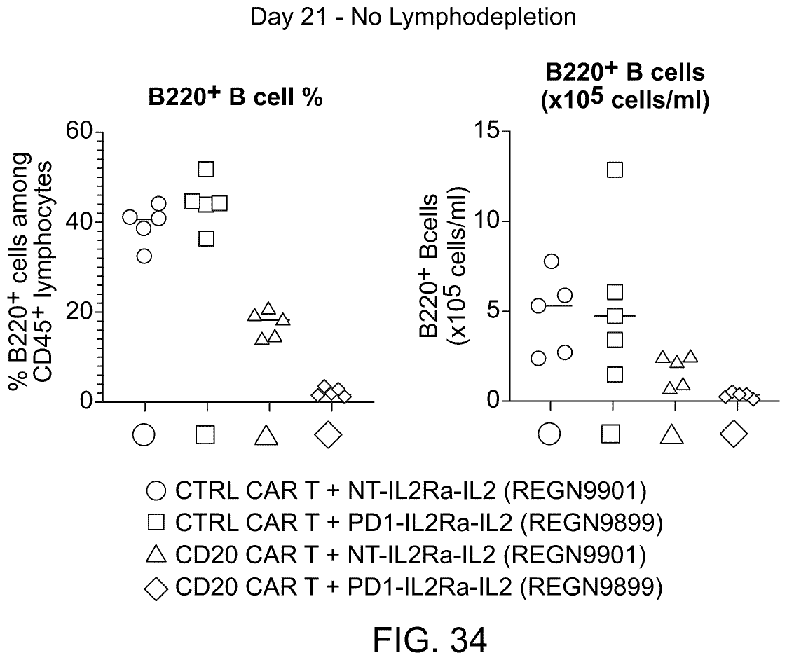

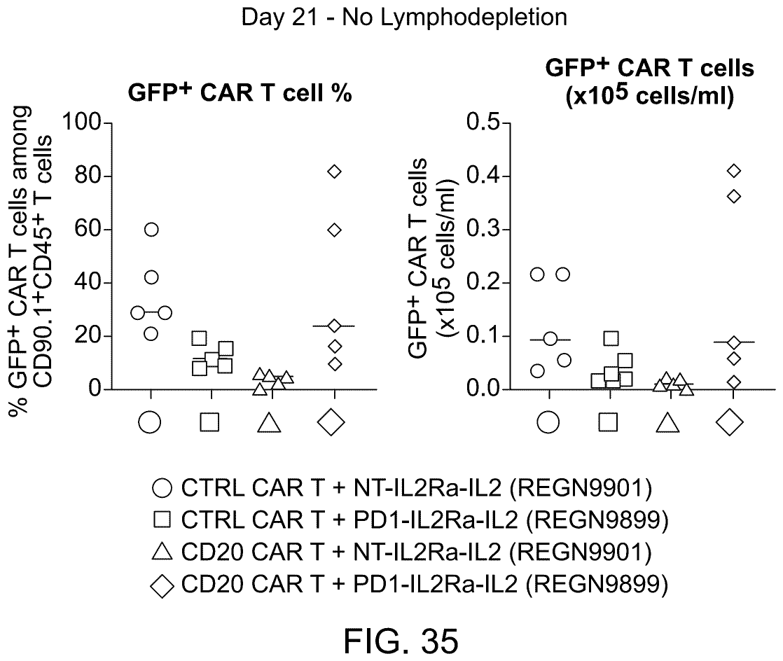

On day 21 in mice that received lymphodepletion, B220⁺ B cell frequencies and absolute numbers in mice receiving huCD20 CAR T+REGN9899 had returned to equivalent levels as mice receiving CTRL CAR T (Table 27; FIG. 32). However, B220⁺ B cell frequencies (p=0.0005) and absolute numbers (p=0.0003) were significantly decreased in mice receiving huCD20 CAR T+REGN9899 compared to mice receiving huCD20 CAR T+REGN9901 (two-tailed, unpaired T-test; Table 27). These results demonstrate that combination of REGN9899 with huCD20 CAR T cells drives prolonged B cell depletion in the context of lymphodepletion.

On day 21 in mice that did not receive lymphodepletion, B220⁺ B cell frequencies (p<0.0001) and absolute numbers (p=0.0056) were also significantly decreased in mice receiving huCD20 CAR T+REGN9899 compared to mice receiving huCD20 CAR T+REGN9901 (two-tailed, unpaired T-test; Table 28). These results demonstrate that combination of REGN9899 with huCD20 CAR T cells drives prolonged B cell depletion, even when no lymphodepletion is administered.

Further, on day 21, the frequencies (p=0.0385) and absolute numbers (p=0.0685) of peripheral blood huCD20 CAR T cells were increased in non-lymphodepleted mice receiving huCD20 CAR T+REGN9899 compared to mice receiving huCD20 CAR T+REGN9901 (Table 28). Thus, even when no lymphodepletion is administered, co-treatment with REGN9899 drove enhanced peripheral CAR T cell expansion compared to REGN9901-treated mice.

Day 21 summary: At this late timepoint, huCD20 CAR T-mediated B cell depletion was superior in mice co-treated with REGN9899 compared to mice co-treated with the control REGN9901, regardless of whether the mice were lymphodepleted. Further, co-treatment with REGN9899 drove enhanced peripheral CAR T cell expansion compared to REGN9901-treated mice in mice that were no lymphodepleted. These results demonstrate that the combination of REGN9899 with CAR T cells drives prolonged CAR T cell functional activity (measured by B cell depletion) and expansion/persistence in vivo, compared to CAR T alone.

Table 27 sets forth frequency and absolute number of peripheral blood B220⁺ B cells compared to CTRL GFP⁺ CAR T in lymphodepleted mice.

TABLE 27
Frequency and absolute number of peripheral blood B220⁺ B cells
compared to CTRL GFP⁺ CAR T in lymphodepleted mice

| | Lymphodepletion | Lymphodepletion | Lymphodepletion | Lymphodepletion |
|---|---|---|---|---|
| | CTRL CAR T + | CTRL CAR T + | CD20 CAR T + | CD20 CAR T + |
| Day | NT-IL2Ra-IL2 | PD1-IL2Ra-IL2 | NT-IL2Ra-IL2 | PD1-IL2Ra-IL2 |
| Mean +/− SEM | (REGN9901) | (REGN9899) | (REGN9901) | (REGN9899) |
| Day 7 | 23.62 ± 1.88 | 16.16 ± 1.06 | 1.19 ± 0.17 | 0.57 ± 0.05 |
| % B220⁺ cells | | | | |
| among CD45⁺ | | | | |
| lymphocytes | | | | |
| Day 7 | 1.72 ± 0.16 | 1.80 ± 0.18 | 0.06 ± 0.01 | 0.06 ± 0.01 |
| B220⁺ B cells | | | | |
| (×10\hat{ }5 cells/ml) | | | | |
| Day 7 | 4.27 ± 0.32 | 2.01 ± 0.07 | 31.60 ± 4.17 | 68.12 ± 3.02 |
| % GFP⁺ CAR T | | | | |
| cells among | | | | |
| CD90.1⁺CD45+ | | | | |
| T cells | | | | |
| Day 7 | 0.31 ± 0.03 | 0.22 ± 0.02 | 1.87 ± 0.51 | 7.19 ± 1.06 |
| GFP⁺ CAR T | | | | |
| cells (×10\hat{ }5 | | | | |
| cells/ml) | | | | |
| Day 21 | 41.64 ± 1.72 | 45.14 ± 1.21 | 39.94 ± 1.49 | 9.31 ± 5.22 |
| % B220⁺ cells | | | | |
| among CD45⁺ | | | | |
| lymphocytes | | | | |
| Day 21 | 2.59 ± 0.34 | 4.88 ± 0.49 | 3.58 ± 0.32 | 0.62 ± 0.36 |
| B220⁺ B cells | | | | |
| (×10\hat{ }5 cells/ml) | | | | |
| Day 21 | 1.89 ± 0.24 | 0.97 ± 0.12 | 0.11 ± 0.04 | 0.46 ± 0.28 |
| % GFP⁺ CAR T | | | | |
| cells among | | | | |
| CD90.1⁺CD45⁺ | | | | |
| T cells | | | | |
| Day 21 | 0.12 ± 0.02 | 0.10 ± 0.01 | 0.01 ± 0.00 | 0.04 ± 0.03 |
| GFP⁺ CAR T | | | | |
| cells (×10\hat{ }5 | | | | |
| cells/ml) | | | | |

Table 28 sets forth the frequency and absolute number of peripheral blood B220+ B cells compared to CTRL GFP+ CAR T in non-lymphodepleted mice.

TABLE 28
Frequency and absolute number of peripheral blood B220⁺ B cells
compared to CTRL GFP⁺ CAR T in non-lymphodepleted mice

| | CTRL CAR T + | CTRL CAR T + | CD20 CAR T + | CD20 CAR T + |
|---|---|---|---|---|
| Day | NT-IL2Ra-IL2 | PD1-IL2Ra-IL2 | NT-IL2Ra-IL2 | PD1-IL2Ra-IL2 |
| Mean +/− SEM | (REGN9901) | (REGN9899) | (REGN9901) | (REGN9899) |
| Day 7 | 37.60 ± 0.87 | 27.48 ± 2.14 | 0.77 ± 0.08 | 1.04 ± 0.29 |
| % B220⁺ cells | | | | |
| among CD45⁺ | | | | |
| lymphocytes | | | | |
| Day 7 | 5.22 ± 0.47 | 4.49 ± 0.95 | 0.08 ± 0.01 | 0.24 ± 0.08 |
| B220⁺ B cells | | | | |
| (×10\hat{ }5 cells/ml) | | | | |
| Day 7 | 1.35 ± 0.10 | 0.85 ± 0.10 | 11.72 ± 0.30 | 46.12 ± 13.11 |
| % GFP⁺ CAR T | | | | |
| cells among | | | | |
| CD90.1⁺CD45⁺ | | | | |
| T cells | | | | |
| Day 7 | 0.18 ± 0.01 | 0.14 ± 0.04 | 1.16 ± 0.16 | 27.02 ± 15.56 |
| GFP⁺ CAR T | | | | |
| cells (×10\hat{ }5 | | | | |
| cells/ml) | | | | |
| Day 21 | 39.42 ± 1.98 | 44.18 ± 2.39 | 17.54 ± 1.27 | 2.08 ± 0.44 |
| % B220⁺ cells | | | | |
| among CD45⁺ | | | | |
| lymphocytes | | | | |
| Day 21 | 4.80 ± 1.01 | 5.75 ± 1.95 | 1.79 ± 0.39 | 0.31 ± 0.07 |
| B220⁺ B cells | | | | |
| (×10\hat{ }5 cells/ml) | | | | |
| Day 21 | 0.90 ± 0.17 | 0.31 ± 0.05 | 0.09 ± 0.03 | 0.96 ± 0.35 |
| % GFP⁺ CAR T | | | | |
| cells among | | | | |
| CD90.1⁺CD45⁺ | | | | |
| T cells | | | | |
| Day 21 | 0.12 ± 0.04 | 0.04 ± 0.01 | 0.01 ± 0.00 | 0.19 ± 0.08 |
| GFP⁺ CAR T | | | | |
| cells (×10\hat{ }5 | | | | |
| cells/ml) | | | | |

### Example 5: Synergistic Anti-Tumor Efficacy of P01-Targeted IL-2 Immunocytokine (P01-IL2Ra-IL2) Treatment in Combination with an Anti-huMUC16 CAR T Cell Therapy

This example relates to an in vivo study performed to demonstrate the anti-tumor efficacy of a PD1-targeted IL-2 immunocytokine (PD1-IL2Ra-IL2) in combination with an anti-huMUC16 CAR T cell therapy.

A syngeneic tumor study was performed in immunocompetent C57BL/6 mice humanized for MUC16 expression. Because these animals express murine PD1, a surrogate PD1-IL2Ra-IL2 reagent was used (i.e., REGN9899, Table 25), which binds to murine PD-1. The mouse PD1 binding moiety is derived from rat anti-mPD-1 clone RMP1-14, and a corresponding non-targeting NT-IL2Ra-IL2 reagent was used (i.e., REGN9901, Table 26).

To generate murine anti-huMUC16 CAR T cells, CD3⁺ T cells were isolated from the spleens of huCD3/huMUC16 knock-in mice using an untouched mouse T-cell isolation kit (Invitrogen #114130) before activation with SG3/GR28 Dynabeads (Invitrogen #111610) and recombinant human IL-2 (20 U/ml; Peprotech #200-02). After 16 hours, the T-cells were transduced via spin infection on plates coated with Retronectin (Takara #T100B) with retrovirus encoding an anti-huMUC16 CAR containing murine CD3z and human 4-1BB intracellular signaling domains. CAR T cells that bind an irrelevant antigen were used as controls.

Table 29 sets forth the amino acid sequences of the anti-huMUC16 and irrelevant-antigen control CAR constructs used in this study.

TABLE 29
Amino acid sequences of anti-huMUC16
and irrelevant-antigen control CAR
constructs

| SEQ | | |
|---|---|---|
| ID | | |
| NO | AMINO ACID SEQUENCE | INFORMATION |
| 57 | MGVPTQLLGLLLLWITDAICEIVLT | anti-huMUC16 |
| | QSPDTLSLSPGERATLSCRASQSLS | CAR-T with |
| | SNYLAWYRQKPGQAPRLLIYGISSR | mouse CD8 |
| | ATGIPDRFSGSGSGTDFTLTISRLE | hinge/ |
| | PEDFAVYYCQQYGSSPWTFGQGTKV | transmembrane, |
| | EIKGGGGSGGGGSGGGGSQVQLVES | human |
| | GGGVVQPGRSLRLSCVASGFTFSNY | 4-1BB and |
| | GIHWVRQAPGKGLEWVAVISDDGSF | mouse CD3z |
| | KFYADSVKGRFTISRDNSKNTLYLQ | signaling |
| | MNSLRVEDSAVYHCAKWQHNWNDGG | domains, and |
| | FDYWGQGTLVTVSSTTTKPVLRTPS | Katushka |
| | PVHPTGTSQPQRPEDCRPRGSVKGT | fluorescent |
| | GLDFACDIYIWAPLAGICVALLLSL | reporter |
| | IITLICYHRSRKRGRKKLLYIFKQP | |
| | FMRPVQTTQEEDGCSCRFPEEEEGG | |
| | CELRAKFSRSAETAANLQDPNQLYN | |
| | ELNLGRREEYDVLEKKRARDPEMGG | |
| | KQQRRRNPQEGVYNALQKDKMAEAY | |
| | SEIGTKGERRRGKGHDGLYQGLSTA | |
| | TKDTYDALHMQTLAPRGSGATNFSL | |
| | LKQAGDVEENPGPMVGEDSVLITEN | |
| | MHMKLYMEGTVNDHHFKCTSEGEGK | |
| | PYEGTQTMKIKVVEGGPLPFAFDIL | |
| | ATSFMYGSKTFINHTQGIPDFFKQS | |
| | FPEGFTWERITTYEDGGVLTATQDT | |
| | SLQNGCLIYNVKINGVNFPSNGPVM | |
| | QKKTLGWEASTEMLYPADSGLRGHA | |
| | QMALKLVGGGYLHCSLKTTYRSKKP | |
| | AKNLKMPGFYFVDRRLERIKEADKE | |
| | TYVEQHEMAVARYCDLPSKLGHS | |
| 58 | EIVLTQSPDTLSLSPGERATLSCRA | Anti-huMUC16 |
| | SQSLSSNYLAWYRQKPGQAPRLLIY | VK |
| | GISSRATGIPDRFSGSGSGTDFTLT | |
| | ISRLEPEDFAVYYCQQYGSSPWTFG | |
| | QGTKVEIK | |
| 59 | QVQLVESGGGVVQPGRSLRLSCVAS | Anti-CD20 VH |
| | GFTFSNYGIHWVRQAPGKGLEWVAV | |
| | ISDDGSFKFYADSVKGRFTISRDNS | |
| | KNTLYLQMNSLRVEDSAVYHCAKWQ | |
| | HNWNDGGFDYWGQGTLVTVSS | |
| 52 | TTTKPVLRTPSPVHPTGTSQPQRPE | Mouse CD8 |
| | DCRPRGSVKGTGLDFACDIYIWAPL | hinge/ |
| | AGICVALLLSLIITLICYHRSR | transmembrane |
| 60 | KRGRKKLLYIFKQPFMRPVQTTQEE | Human 4-1BB |
| | DGCSCRFPEEEEGGCEL | signaling |
| | | domain |
| 54 | RAKFSRSAETAANLQDPNQLYNELN | Mouse CD3z |
| | LGRREEYDVLEKKRARDPEMGGKQQ | signaling |
| | RRRNPQEGVYNALQKDKMAEAYSEI | domain |
| | GTKGERRRGKGHDGLYQGLSTATKD | |
| | TYDALHMQTLAPR | |
| 61 | MVGEDSVLITENMHMKLYMEGTVND | Katushka |
| | HHFKCTSEGEGKPYEGTQTMKIKVV | fluorescent |
| | EGGPLPFAFDILATSFMYGSKTFIN | reporter |
| | HTQGIPDFFKQSFPEGFTWERITTY | |
| | EDGGVLTATQDTSLQNGCLIYNVKI | |
| | NGVNFPSNGPVMQKKTLGWEASTEM | |
| | LYPADSGLRGHAQMALKLVGGGYLH | |
| | CSLKTTYRSKKPAKNLKMPGFYFVD | |
| | RRLERIKEADKETYVEQHEMAVARY | |
| | CDLPSKLGHS | |
| | MGVPTQLLGLLLLWITDAICEIVMT | Control CAR T |
| | QSPATLSVSPGERATLSCRASQSVS | with mouse |
| | SNLAWYQQKPGQAPRLLIYGTSTRA | CD28 |
| | TGIPARFSGSGSGTEFTLTISSLQS | hinge/ |
| | EDFAVYYCQQYNNWPLTFGGGTKVE | transmembrane/ |
| | IKGGGGSGGGGSGGGGSEVQLVESG | signaling |
| | GGLVQPGRSLRLSCVASGFTFNDYA | and mouse |
| | MHWVRQAPGKGLEWVSVISWNSDSI | CD3z signaling |
| | GYADSVKGRFTISRDNAKNSLYLQM | domain and |
| | HSLRAEDTALYYCAKDNHYGSGSYY | GFP reporter |
| | YYQYGMDVWGQGTTVTVSSGGGGSI | |
| | EFMYPPPYLDNERSNGTIIHIKEKH | |
| | LCHTQSSPKLFWALVVVAGVLFCYG | |
| | LLVTVALCVIWTNSRRNRGGQSDYM | |
| | NMTPRRPGLTRKPYQPYAPARDFAA | |
| | YRPRAKFSRSAETAANLQDPNQLYN | |
| | ELNLGRREEYDVLEKKRARDPEMGG | |
| | KQQRRRNPQEGVYNALQKDKMAEAY | |
| | SEIGTKGERRRGKGHDGLYQGLSTA | |
| | TKDTYDALHMQTLAPRGSGATNFSL | |
| | LKQAGDVEENPGPMVSKGEELFTGV | |
| | VPILVELDGDVNGHKFSVSGEGEGD | |
| | ATYGKLTLKFICTTGKLPVPWPTLV | |
| | TTLTYGVQCFSRYPDHMKQHDFFKS | |
| | AMPEGYVQERTIFFKDDGNYKTRAE | |
| | VKFEGDTLVNRIELKGIDFKEDGNI | |
| | LGHKLEYNYNSHNVYIMADKQKNGI | |
| | KVNFKIRHNIEDGSVQLADHYQQNT | |
| | PIGDGPVLLPDNHYLSTQSALSKDP | |
| | NEKRDHMVLLEFVTAAGITLGMDEL | |
| | YK | |
| 44 | EIVMTQSPATLSVSPGERATLSCRA | CTRL mAb |
| | SQSVSSNLAWYQQKPGQAPRLLIYG | (anti-huCD20) |
| | TSTRATGIPARFSGSGSGTEFTLTI | VK |
| | SSLQSEDFAVYYCQQYNNWPLTFGG | |
| | GTKVEIK | |
| 45 | EVQLVESGGGLVQPGRSLRLSCVAS | CTRL mAb |
| | GFTFNDYAMHWVRQAPGKGLEWVSV | (anti-huCD20) |
| | ISWNSDSIGYADSVKGRFTISRDNA | VH |
| | KNSLYLQMHSLRAEDTALYYCAKDN | |
| | HYGSGSYYYYQYGMDVWGQGTTVTV | |
| | SS | |
| 62 | IEFMYPPPYLDNERSNGTIIHIKEK | Mouse CD28 |
| | HLCHTQSSPKLFWALVVVAGVLFCY | hinge/ |
| | GLLVTVALCVIWTNSRRNRGGQSDY | transmembrane/ |
| | MNMTPRRPGLTRKPYQPYAPARDFA | signaling |
| | AYRP | |
| 54 | RAKFSRSAETAANLQDPNQLYNELN | Mouse CD3z |
| | LGRREEYDVLEKKRARDPEMGGKQQ | signaling |
| | RRRNPQEGVYNALQKDKMAEAYSEI | domain |
| | GTKGERRRGKGHDGLYQGLSTATKD | |
| | TYDALHMQTLAPR | |
| 56 | MVSKGEELFTGVVPILVELDGDVNG | GFP |
| | HKFSVSGEGEGDATYGKLTLKFICT | |
| | TGKLPVPWPTLVTTLTYGVQCFSRY | |
| | PDHMKQHDFFKSAMPEGYVQERTIF | |
| | FKDDGNYKTRAEVKFEGDTLVNRIE | |
| | LKGIDFKEDGNILGHKLEYNYNSHN | |
| | VYIMADKQKNGIKVNFKIRHNIEDG | |
| | SVQLADHYQQNTPIGDGPVLLPDNH | |
| | YLSTQSALSKDPNEKRDHMVLLEFV | |
| | TAAGITLGMDELYK | |

MUC16-humanized mice were lymphodepleted with a sublethal dose of total body irradiation (400 cGy) one day before subcutaneous implantation with 10×10⁶ ID8/VEGF/huMUC16 tumor cells in the right flank. One day after tumor implantation, mice were injected intravenously with 4×10⁶ CAR⁺ anti-huMUC16 CAR T or control CAR T cells. The same day, the mice received either PD1-IL2Ra-IL2 (REGN9899, Table 25) or a control, non-targeting NT-IL2Ra-IL2 (REGN9901, Table 26) intraperitoneally at 1 mg/kg. Two days post-CAR T cell injection, the mice received one additional dose of PD1-targeted or control immunocytokine at 1 mg/kg. Tumor growth was assessed over 43 days via twice-weekly caliper measurements and calculated by the following formula: (length×width²)/2.

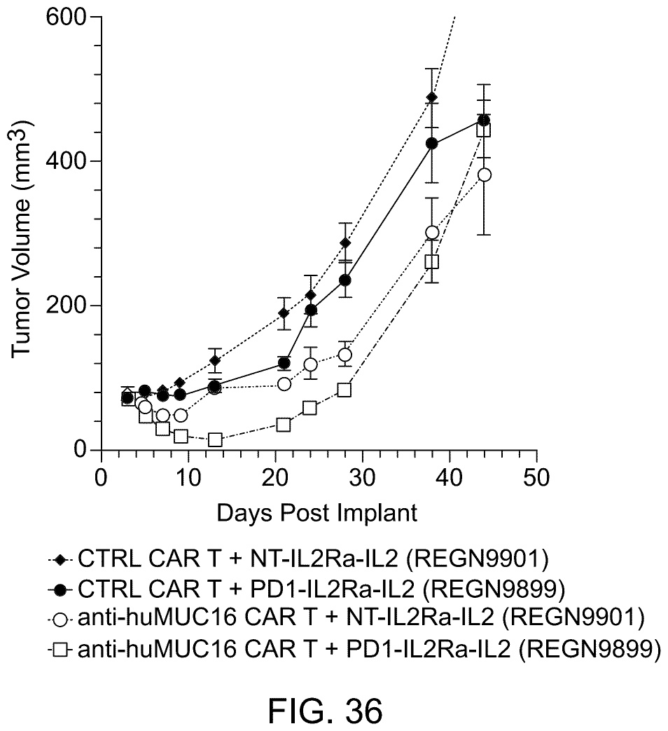

Results: Similar tumor growth was noted in animals receiving CTRL CAR T and either REGN9901 or REGN9899, as well as animals that received anti-huMUC16 CAR T and REGN9901 (Table 30; FIG. 36). However, tumor growth was significantly inhibited in mice receiving anti-huMUC16 CAR T-cells combined with REGN9899 (Table 30; FIG. 36).

Two-way ANOVA P values for anti-huMUC16 CAR T+REGN9899 vs. CTRL CAR T+REGN9901 are the following: Day 13: p=0.003; Day 21: p<0.0001; Day 24: p<0.0001; Day 28: p<0.0001. Two-way ANOVA P values for anti-huMUC16 CAR T+REGN9899 vs. CTRL CAR T+REGN9901 are the following: Day 21: p=0.0368; Day 24: p=0.0001; Day 28: p<0.0001. Note: Two mice from the “anti-huMUC16 CAR T+REGN9899” group died after the Day −7 measurement due to circumstances unrelated to the study or therapeutic agents.

Table 30 sets forth the tumor volume+/−SEM and number of live mice at specific days with specific antibody treatments.

TABLE 30
Tumor volume +/− SEM and number of live mice at
specific days with specific antibody treatments

| | | | |
|---|---|---|---|
| | Mean tumor | Tumor | Number |
| | volume | volume | of live |
| Antibody Treatment | (mm³) | SEM | mice |

DAY 3

| | | | |
|---|---|---|---|
| CTRL CAR T + | 67.63 | 5.48 | 7 of 7 |
| NT-IL2Ra-IL2 (REGN9901) | | | |
| CTRL CAR T + | 78.81 | 9.88 | 7 of 7 |
| PD1-IL2Ra-IL2 (REGN9899) | | | |
| anti-huMUC16 CAR T + | 76.18 | 8.14 | 7 of 7 |
| NT-IL2Ra-IL2 (REGN9901) | | | |
| anti-huMUC16 CAR T + | 69.95 | 6.76 | 10 of 10 |
| PD1-IL2Ra-IL2 (REGN9899) | | | |

DAY 5

| | | | |
|---|---|---|---|
| CTRL CAR T + | 73.97 | 5.42 | 7 of 7 |
| NT-IL2Ra-IL2 (REGN9901) | | | |
| CTRL CAR T + | 82.52 | 5.73 | 7 of 7 |
| PD1-IL2Ra-IL2 (REGN9899) | | | |
| anti-huMUC16 CAR T + | 59.47 | 7.87 | 7 of 7 |
| NT-IL2Ra-IL2 (REGN9901) | | | |
| anti-huMUC16 CAR T + | 46.01 | 3.61 | 10 of 10 |
| PD1-IL2Ra-IL2 (REGN9899) | | | |

DAY 7

| | | | |
|---|---|---|---|
| CTRL CAR T + | 82.33 | 7.39 | 7 of 7 |
| NT-IL2Ra-IL2 (REGN9901) | | | |
| CTRL CAR T + | 75.66 | 6.22 | 7 of 7 |
| PD1-IL2Ra-IL2 (REGN9899) | | | |
| anti-huMUC16 CAR T + | 48.55 | 3.56 | 7 of 7 |
| NT-IL2Ra-IL2 (REGN9901) | | | |
| anti-huMUC16 CAR T + | 29.17 | 1.85 | 10 of 10 |
| PD1-IL2Ra-IL2 (REGN9899) | | | |

DAY 9

| | | | |
|---|---|---|---|
| CTRL CAR T + | 93.00 | 4.70 | 7 of 7 |
| NT-IL2Ra-IL2 (REGN9901) | | | |
| CTRL CAR T + | 76.48 | 6.72 | 7 of 7 |
| PD1-IL2Ra-IL2 (REGN9899) | | | |
| anti-huMUC16 CAR T + | 47.76 | 8.37 | 5 of 7 |
| NT-IL2Ra-IL2 (REGN9901) | | | |
| anti-huMUC16 CAR T + | 19.93 | 1.98 | 10 of 10 |
| PD1-IL2Ra-IL2 (REGN9899) | | | |

DAY 13

| | | | |
|---|---|---|---|
| CTRL CAR T + | 124.64 | 18.19 | 7 of 7 |
| NT-IL2Ra-IL2 (REGN9901) | | | |
| CTRL CAR T + | 89.31 | 9.15 | 7 of 7 |
| PD1-IL2Ra-IL2 (REGN9899) | | | |
| anti-huMUC16 CAR T + | 86.35 | 8.65 | 5 of 7 |
| NT-IL2Ra-IL2 (REGN9901) | | | |
| anti-huMUC16 CAR T + | 15.66 | 2.69 | 10 of 10 |
| PD1-IL2Ra-IL2 (REGN9899) | | | |

DAY 21

| | | | |
|---|---|---|---|
| CTRL CAR T + | 189.48 | 21.21 | 7 of 7 |
| NT-IL2Ra-IL2 (REGN9901) | | | |
| CTRL CAR T + | 121.08 | 11.51 | 7 of 7 |
| PD1-IL2Ra-IL2 (REGN9899) | | | |
| anti-huMUC16 CAR T + | 90.65 | 7.75 | 5 of 7 |
| NT-IL2Ra-IL2 (REGN9901) | | | |
| anti-huMUC16 CAR T + | 36.29 | 3.82 | 10 of 10 |
| PD1-IL2Ra-IL2 (REGN9899) | | | |

DAY 24

| | | | |
|---|---|---|---|
| CTRL CAR T + | 216.10 | 26.94 | 7 of 7 |
| NT-IL2Ra-IL2 (REGN9901) | | | |
| CTRL CAR T + | 195.06 | 23.64 | 7 of 7 |
| PD1-IL2Ra-IL2 (REGN9899) | | | |
| anti-huMUC16 CAR T + | 119.96 | 22.81 | 5 of 7 |
| NT-IL2Ra-IL2 (REGN9901) | | | |
| anti-huMUC16 CAR T + | 59.02 | 7.62 | 10 of 10 |
| PD1-IL2Ra-IL2 (REGN9899) | | | |

DAY 28

| | | | |
|---|---|---|---|
| CTRL CAR T + | 288.42 | 27.49 | 7 of 7 |
| NT-IL2Ra-IL2 (REGN9901) | | | |
| CTRL CAR T + | 235.84 | 21.67 | 7 of 7 |
| PD1-IL2Ra-IL2 (REGN9899) | | | |
| anti-huMUC16 CAR T + | 134.46 | 16.75 | 5 of 7 |
| NT-IL2Ra-IL2 (REGN9901) | | | |
| anti-huMUC16 CAR T + | 83.48 | 6.80 | 10 of 10 |
| PD1-IL2Ra-IL2 (REGN9899) | | | |

DAY 38

| | | | |
|---|---|---|---|
| CTRL CAR T + | 489.58 | 38.35 | 7 of 7 |
| NT-IL2Ra-IL2 (REGN9901) | | | |
| CTRL CAR T + | 424.91 | 56.07 | 7 of 7 |
| PD1-IL2Ra-IL2 (REGN9899) | | | |
| anti-huMUC16 CAR T + | 301.17 | 46.48 | 5 of 7 |
| NT-IL2Ra-IL2 (REGN9901) | | | |
| anti-huMUC16 CAR T + | 261.00 | 29.05 | 10 of 10 |
| PD1-IL2Ra-IL2 (REGN9899) | | | |

DAY 44

| | | | |
|---|---|---|---|
| CTRL CAR T + | 732.83 | 57.31 | 6 of 7 |
| NT-IL2Ra-IL2 (REGN9901) | | | |
| CTRL CAR T + | 456.79 | 50.69 | 7 of 7 |
| PD1-IL2Ra-IL2 (REGN9899) | | | |
| anti-huMUC16 CAR T + | 382.30 | 84.01 | 5 of 7 |
| NT-IL2Ra-IL2 (REGN9901) | | | |
| anti-huMUC16 CAR T + | 444.98 | 41.39 | 10 of 10 |
| PD1-IL2Ra-IL2 (REGN9899) | | | |

### Example 6: Synergistic Anti-Tumor Efficacy of PD1-Targeted IL-2 Immunocytokine (PD1-IL2Ra-IL2) Treatment in Combination with an Anti-huMUC16 CAR T Cell Therapy

This example relates to an in vivo study performed to demonstrate the synergistic anti-tumor efficacy of PD1-targeted IL-2 immunocytokine (PD1-IL2Ra-IL2) treatment in combination with an anti-huMUC16 CAR T cell therapy.

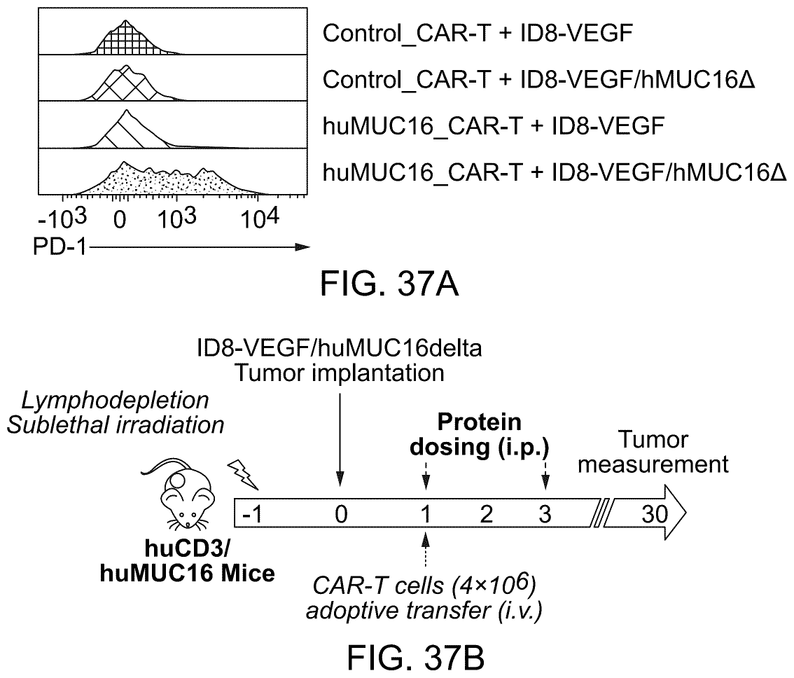

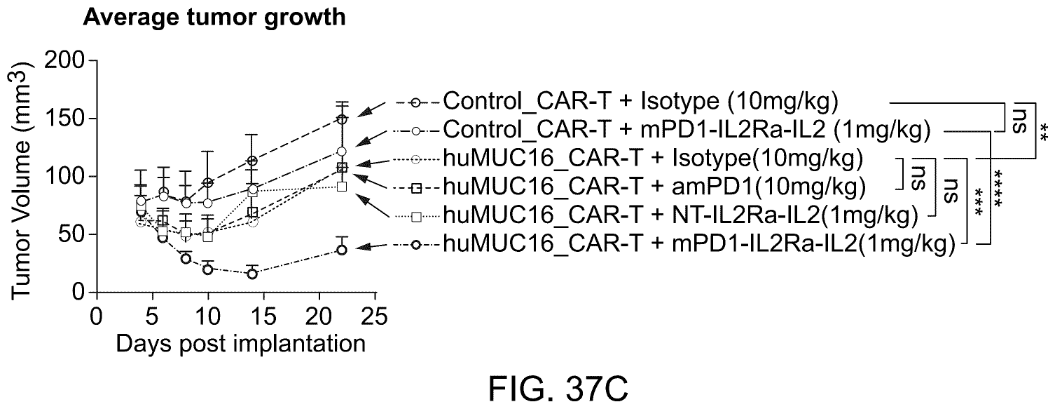

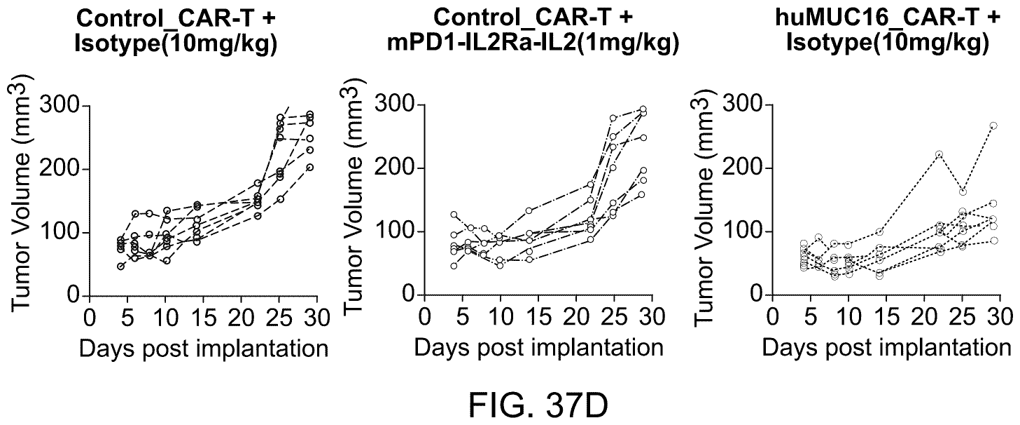

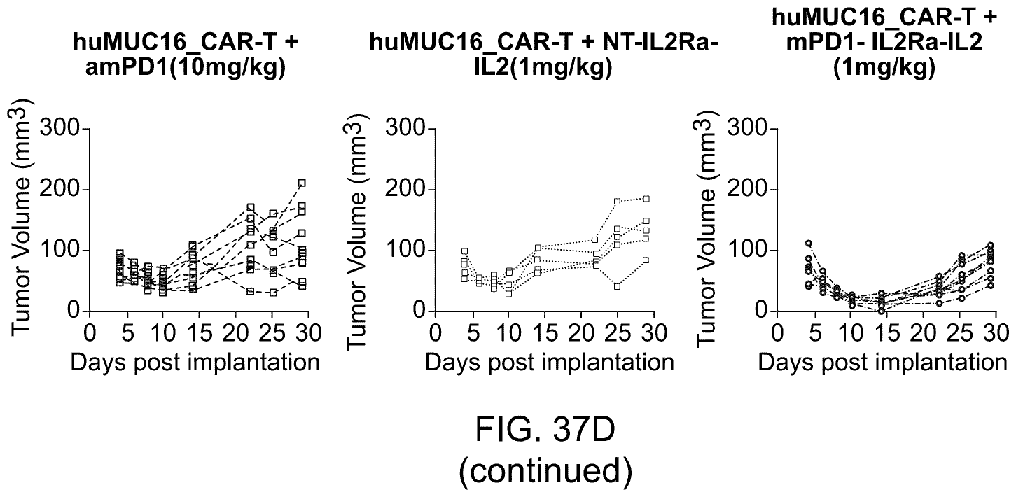

Despite being an effective therapy for some hematological malignancies, the therapeutic activity of CAR-T cells has been limited in most solid tumors, in part due to poor in vivo persistence and functionality. Numerous combination strategies are being explored to overcome these limitations of CAR-T cells in solid tumors (Young et al., Cancer Discovery, 12:1625-1633 (2022); Al-Haideri et al., Cancer Cell International, 22:365 (2022). To test if PD1-IL2Ra-IL2 improves the anti-tumor activity of CAR-T cells in solid tumors, an evaluation was conducted regarding the combinatorial efficacy of anti-huMUC16 CAR-T cells+mPD1-IL2Ra-IL2 in controlling syngeneic ID8-VEGF/huMUC16-delta tumors, since anti-huMUC16 CAR-T cells upregulate PD-1 expression upon co-culture with target cells expressing huMUC16 (FIG. 37A). CD3/MUC16 double-humanized mice were lymphodepleted, implanted with ID8-VEGF/huMUC16-delta tumor cells, and treated with either anti-huMUC16 or control CAR-T cells in combination with mPD1-IL2Ra-IL2 or control molecules on the indicated days (FIG. 37B). Compared to control CAR-T cells+isotype mAb, huMUC16 CAR-T cells+isotype mAb treatment modestly delayed tumor growth. This single agent efficacy of huMUC16 CAR-T cells was not further improved when they were combined with either NT-IL2Ra-IL2 or high dose anti-mPD1. In contrast, combination of huMUC16 CAR-T cells with mPD1-IL2Ra-IL2 resulted in significantly enhanced anti-tumor efficacy, with tumor regression observed in all mice in this treatment group. There was no therapeutic benefit of mPD1-IL2Ra-IL2 in mice that received control CAR-T cells, suggesting that in these lymphodepleted mice the activity of mPD1-IL2Ra-IL2 is dependent on transferred huMUC16 CAR-T cells (FIGS. 37C and 37D). Collectively these results demonstrate that PD1-IL2Ra-IL2 enhances the in vivo anti-tumor activity of CAR-T cells.

The present disclosure is not to be limited in scope by the specific embodiments described herein. Indeed, various modifications of the disclosure in addition to those described herein will become apparent to those skilled in the art from the foregoing description and accompanying figures. Such modifications are intended to fall within the scope of the appended claims.

1. A method for increasing the efficacy of adoptive cell therapy (ACT), comprising:
(a) selecting a subject with cancer; and
(b) administering to the subject a therapeutically effective amount of an ACT in combination with a therapeutically effective amount of a targeted immunocytokine,
wherein the targeted immunocytokine is a fusion protein comprising (a) an immunoglobulin antigen-binding domain of a checkpoint inhibitor and (b) an IL2 moiety, and
wherein administration of the combination leads to increased efficacy and duration of anti-tumor response, as compared to a subject treated with the ACT as monotherapy.

2. A method for treating cancer, comprising administering to a subject in need thereof a therapeutically effective amount of an adoptive cell therapy (ACT) in combination with a therapeutically effective amount of a targeted immunocytokine, wherein administration of the combination leads to increased efficacy and duration of anti-tumor response, as compared to a subject treated with the ACT as monotherapy.

3. The method of claim 1, wherein the ACT comprises an immune cell selected from a T cell, a tumor-infiltrating lymphocyte, and a natural killer (NK) cell.

4. The method of claim 3, wherein the immune cell comprises a modified T cell receptor (TCR) against a tumor-associated antigen (TAA), or a chimeric antigen receptor (CAR) against a TAA.

5. The method of claim 4, wherein the TAA is selected from AFP, ALK, BAGE proteins, BCMA, BIRC5 (survivin), BIRC7, β-catenin, brc-abl, BRCA1, BORIS, CA9, carbonic anhydrase IX, caspase-8, CALR, CCR5, CD19, CD20 (MS4A1), CD22, CD30, CD40, CDK4, CEA, CTLA4, cyclin-B1, CYP1B1, EGFR, EGFRvIII, ErbB2/Her2, ErbB3, ErbB4, ETV6-AML, EpCAM, EphA2, Fra-1, FOLR1, GAGE proteins, GD2, GD3, GloboH, glypican-3, GM3, gp100, Her2, HLA/B-raf, HLA/k-ras, HLA/MAGE-A3, hTERT, LMP2, MAGE proteins (e.g., MAGE-1, -2, -3, -4, -6, and -12), MART-1, mesothelin, ML-IAP, Muc1, Muc2, Muc3, Muc4, Muc5, Muc16 (CA-125), MUM1, NA17, NY-BR1, NY-BR62, NY-BR85, NY-ESO1, OX40, p15, p53, PAP, PAX3, PAX5, PCTA-1, PLAC1, PRLR, PRAME, PSMA (FOLH1), RAGE proteins, Ras, RGS5, Rho, SART-1, SART-3, STEAP1, STEAP2, TAG-72, TGF-β, TMPRSS2, Thompson-nouvelle antigen (Tn), TRP-1, TRP-2, tyrosinase, and uroplakin-3.

6. (canceled)

7. The method of claim 1, wherein the IL2 moiety comprises (i) IL2 receptor alpha (IL2Ra) or a fragment thereof; and (ii) IL2 or a fragment thereof.

8. The method of claim 1, wherein the checkpoint inhibitor is an inhibitor of PD1, PD-L1, PD-L2, LAG-3, CTLA-4, TIM3, A2aR, B7H1, BTLA, CD160, LAIR1, TIGHT, VISTA, or VTCN1.

9. The method of claim 1, wherein the checkpoint inhibitor is an inhibitor of PD-1.

10. The method of claim 1, wherein the antigen-binding domain comprises a heavy chain variable region (HCVR) comprising an amino acid sequence selected from SEQ ID NOs: 1, 11, and 20; and a light chain variable region (LCVR) comprising an amino acid sequence selected from SEQ ID NOs: 5 and 15.

11. The method of claim 1, wherein the antigen-binding domain comprises three heavy chain complementarity determining regions (CDRs) (HCDR1, HCDR2, and HCDR3) and three light chain CDRs (LCDR1, LCDR2, and LCDR3) wherein HCDR1, HCDR2, HCDR3, LCDR1, LCDR2, and LCDR3 comprise the amino acid sequences selected from:
(a) SEQ ID NOs: 2, 3, 4, 6, 7, and 8, respectively;
(b) SEQ ID NOs: 12, 13, 14, 16, 7, and 17, respectively; and
(c) SEQ ID NOs: 21, 22, 23, 6, 7, and 8, respectively.

12. The method of claim 1, wherein the antigen-binding domain comprises a HCVR/LCVR amino acid sequence pair selected from SEQ ID NOs: 1/5, 11/15, and 20/5.

13. The method of claim 1, wherein the fusion protein comprises a heavy chain comprising a heavy chain variable region (HCVR) and a heavy chain constant region of IgG1 isotype.

14. The method of claim 1, wherein the fusion protein comprises a heavy chain comprising a heavy chain variable region (HCVR) and a heavy chain constant region of IgG4 isotype.

15. The method of claim 1, wherein the fusion protein comprises a heavy chain constant region comprising the amino acid sequence of SEQ ID NO: 26.

16. The method of claim 1, wherein the fusion protein comprises a heavy chain comprising an amino acid sequence selected from SEQ ID NOs: 9, 18, and 24; and a light chain comprising an amino acid sequence selected from SEQ ID NOs: 10, 19, and 25.

17. The method of claim 1, wherein the fusion protein comprises:
(a) a heavy chain comprising the amino acid sequence of SEQ ID NO: 24, and a light chain comprising the amino acid sequence of SEQ ID NO: 25;
(b) a heavy chain comprising the amino acid sequence of SEQ ID NO: 9, and a light chain comprising the amino acid sequence of SEQ ID NO: 10; or
(c) a heavy chain comprising the amino acid sequence of SEQ ID NO: 18, and a light chain comprising the amino acid sequence of SEQ ID NO: 19.

18. The method of claim 1, wherein the antigen-binding domain comprises a heavy chain and the IL2 moiety is attached to the C-terminus of the heavy chain via a linker comprising the amino acid sequence of SEQ ID NO: 30 or 31.

19. The method of claim 1, wherein the IL2 moiety comprises the amino acid sequence of SEQ ID NO: 27.

20. The method of claim 1, wherein the IL2 moiety comprises wild type IL2.

21. The method of claim 20, wherein the IL2 comprises the amino acid sequence of SEQ ID NO: 29.

22. The method of claim 1, wherein the IL2 moiety comprises the IL2 or fragment thereof connected via a linker to the C-terminus of the IL2Ra or fragment thereof.

23. The method of claim 22, wherein the IL2Ra or fragment thereof comprises the amino acid sequence of SEQ ID NO: 28.

24. The method of claim 1, wherein the fusion protein is a dimeric fusion protein that dimerizes through the heavy chain constant region of each monomer.

25. The method of claim 1, wherein the targeted immunocytokine comprises a PD-1 targeting moiety and an IL2 moiety.

26. The method of claim 25, wherein the PD-1 targeting moiety comprises an immunoglobulin antigen-binding domain that binds specifically to PD-1.

27. The method of claim 26, wherein the antigen-binding domain comprises:
(a) a heavy chain variable region (HCVR) comprising the amino acid sequence of SEQ ID NO: 20, and a light chain variable region (LCVR) comprising the amino acid sequence of SEQ ID NO: 5;
(b) a HCVR comprising the amino acid sequence of SEQ ID NO: 1, and a LCVR comprising the amino acid sequence of SEQ ID NO: 5; or
(c) a HCVR comprising the amino acid sequence of SEQ ID NO: 11, and a LCVR comprising the amino acid sequence of SEQ ID NO: 15.

28. The method of claim 25, wherein the IL2 moiety comprises (i) IL2Ra or a fragment thereof; and (ii) IL2 or a fragment thereof.

29. The method of claim 25, wherein the IL2 moiety comprises the amino acid sequence of SEQ ID NO: 27.

30. The method of claim 1, wherein the targeted immunocytokine is REGN10597.

31. The method of claim 1, wherein the cancer is selected from adrenal gland tumors, biliary cancer, bladder cancer, brain cancer, breast cancer, carcinoma, central or peripheral nervous system tissue cancer, cervical cancer, colon cancer, endocrine or neuroendocrine cancer or hematopoietic cancer, esophageal cancer, fibroma, gastrointestinal cancer, glioma, head and neck cancer, Li-Fraumeni tumors, liver cancer, lung cancer, lymphoma, melanoma, meningioma, neuroendocrine type I or type II tumors, multiple myeloma, myelodysplastic syndromes, myeloproliferative diseases, nasopharyngeal cancer, oral cancer, oropharyngeal cancer, osteogenic sarcoma tumors, ovarian cancer, pancreatic cancer, pancreatic islet cell cancer, parathyroid cancer, pheochromocytoma, pituitary tumor, prostate cancer, rectal cancer, renal cancer, respiratory cancer, sarcoma, skin cancer, stomach cancer, testicular cancer, thyroid cancer, tracheal cancer, urogenital cancer, and uterine cancer.

32. The method of claim 1, wherein administration of the combination produces a therapeutic effect selected from one or more of: delay in tumor growth, reduction in tumor cell number, tumor regression, increase in survival, partial response, and complete response.

33. The method of claim 1, wherein the therapeutically effective amount of the ACT comprises 1×10⁶ or more immune cells.

34. The method of claim 1, wherein the therapeutically effective amount of the targeted immunocytokine is 0.005 mg/kg to 10 mg/kg of the subject's body weight.

35. The method of claim 1, wherein the targeted immunocytokine is administered intravascularly, subcutaneously, intraperitoneally, or intratumorally.

36. The method of claim 1, wherein the ACT is administered via intravenous infusion.

37. The method of claim 1, wherein the ACT is administered before or after administration of the targeted immunocytokine.

38. The method of claim 1, wherein the ACT is administered concurrently with administration of the targeted immunocytokine.

39. The method of claim 1, wherein the targeted immunocytokine and/or the ACT is administered in one or more doses to the subject.

40. The method of claim 1, further comprising administering an additional therapeutic agent or therapy to the subject.

41. The method of claim 40, wherein the additional therapeutic agent or therapy is selected from radiation, surgery, a chemotherapeutic agent, a cancer vaccine, a B7-H3 inhibitor, a B7-H4 inhibitor, a lymphocyte activation gene 3 (LAG3) inhibitor, a T cell immunoglobulin and mucin-domain containing-3 (TIM3) inhibitor, a galectin 9 (GAL9) inhibitor, a V-domain immunoglobulin (Ig)-containing suppressor of T cell activation (VISTA) inhibitor, a Killer-Cell Immunoglobulin-Like Receptor (KIR) inhibitor, a B and T lymphocyte attenuator (BTLA) inhibitor, a T cell immunoreceptor with Ig and ITIM domains (TIGIT) inhibitor, a CD47 inhibitor, an indoleamine-2,3-dioxygenase (IDO) inhibitor, a vascular endothelial growth factor (VEGF) antagonist, an angiopoietin-2 (Ang2) inhibitor, a transforming growth factor beta (TGFβ) inhibitor, an epidermal growth factor receptor (EGFR) inhibitor, an antibody to a tumor-specific antigen, Bacillus Calmette-Guerin vaccine, granulocyte-macrophage colony-stimulating factor (GM-CSF), a cytotoxin, an interleukin 6 receptor (IL-6R) inhibitor, an interleukin 4 receptor (IL-4R) inhibitor, an IL-10 inhibitor, IL-7, IL-12, IL-21, IL-15, an antibody-drug conjugate, an anti-inflammatory drug, and combinations thereof.

42. (canceled)
